(19) United States
(12) Patent Application Publication
(10) Pub. No.: US 2026/0176343 A1
(43) Pub. Date: Jun. 25, 2026

(54) AUTOREGULATION OF THERAPEUTIC AGENTS

(71) Applicants: UCL BUSINESS LTD, London (GB); NOVALGEN LIMITED, Northwood Hills (GB)

(72) Inventors: Vincent Muczynski, London (GB); Amit Nathwani, London (GB)

(73) Assignees: UCL BUSINESS LTD, London (GB); NOVALGEN LIMITED, Northwood Hills (GB)

(21) Appl. No.: 19/126,362

(22) PCT Filed: Nov. 2, 2023
(86) PCT No.: PCT/GB2023/052865

§ 371 Date: May 1, 2025

(30) Foreign Application Priority Data
Nov. 2, 2022 (GB) 2216284.6

(51) Int. Cl. C07K 16/22

(52) U.S. Cl. CPC C07K 16/22; C07K 2317/21; C07K 2317/622; C07K 2317/92

(57) ABSTRACT

The present invention relates to a therapeutic agent comprising an autoregulation element, preferably wherein the therapeutic agent is an antigen binding protein. Methods of treatment comprising use of said agent are also disclosed.

### FIELD OF THE INVENTION

The present invention relates to a therapeutic agent, preferably wherein the therapeutic agent is an antigen binding protein. Methods of treatment comprising use of said agent are also disclosed.

### BACKGROUND OF THE INVENTION

Homeostasis and sensitivity to feedback regulation signals are naturally occurring mechanisms that balance virtually all biological functions within the body. However, most medicines are devoid of such regulatory mechanisms and the technologies to modulate the therapeutic action of a drug in response to physiological or pathological signals are highly sought after but rarely been achieved.

Over the last decade, advances in molecular and cellular engineering have seen the development of complex and powerful drugs including therapeutic monoclonal and bispecific antibodies, Chimeric Antigen Receptor (CAR) T cells and engineered therapeutic peptides that enabled unique functionalities and brought major clinical benefits, particularly for patients with significant unmet needs. The transformative potential of these novel therapies is, however, tempered by the occurrence of serious adverse events linked to their modes of action (MoA), which can include cytokine-mediated toxicities in adoptive immunotherapy; cytokine release syndrome; and thromboembolism and pro-thrombotic risks in haemophilia A. Due to the intrinsic long half-life of some of these therapies (e.g. antibodies, engineered cells), such adverse events represents a serious life-threatening risk for the patient.

### SUMMARY OF THE INVENTION

This invention demonstrates how to harness endogenous signals or factors, which can be activated as a direct response to a therapeutic treatment in abnormal conditions, to create a next-generation of therapeutic agents with built-in “switch” mechanisms to regulate their therapeutic activity when there is emerging risk of severe MoA related toxicity.

The following invention describes the methods to design and develop next-generation therapeutics agents with embedded self-regulation elements (autoregulation elements) that have the capacity to inactivate the agent in the context of excessive or undesired therapeutic, efficacy to reduce the risk of adverse events and increase the safety profile of these agents. The self-regulation elements are small cleavable peptides inserted within the structure of the therapeutic agent in such a way that their cleavage will inactivate the therapeutic agent by breaking it into fragments that are either non-functional or rapidly eliminated (FIG. 1).

The self-regulation peptides are sensitive to the proteolytic cleavage mediated by specifically selected endogenous factors whose concentration, activity or bioavailability is modified in response to treatment-induced adverse events. Thus, when the therapeutic activity of the drug remains within a defined range where no adverse events occur, the drug will retain its full therapeutic potential, while when the therapeutic activity is above a threshold that separate normal biological activity from MoA-related adverse events, the modification in activity of the endogenous factor will lead to cleavage of the self-regulation elements and ultimately inactivate the therapeutic agent to reduce the risk of progression to severe toxicities (FIG. 1).

The proof-of-concept was established with two therapeutic bispecific antibodies. Emicizumab is a marketed humanised bispecific IgG4 for the treatment of patients with haemophilia A with and without inhibitors, and NVG-111 is a bispecific tandem-single chain fragment variable (scFv) investigational new drug currently in Phase I trial for Chronic Leukaemic Lymphomas (CLL) and Mantle Cells Lymphomas (MCL). Application of the autoregulation technology was also demonstrated in a model of CAR-T cell. The present invention define a versatile platform that is target and format agnostic and can be applied to any therapeutic agent, including therapeutic bispecific and monoclonal antigen binding proteins and antibodies including non-canonical formats, therapeutic non-antibody derived peptides as well as engineered cells (including but not limited to CART cells) to extend the therapeutic index of such therapies with a potential to improve effectiveness and safety for a better clinical outcome in patients.

Therefore, the present invention provides:
A therapeutic agent comprising an autoregulation element, wherein the autoregulation element is a small peptide cleavable by an endogenous factor, wherein the activity of the endogenous factor is modified as a consequence of the activity of the therapeutic agent, and wherein cleavage of the autoregulation element results in separation of the therapeutic agent into non-functional fragments.

The invention also provides:
A composition comprising the therapeutic agent as defined herein and a pharmaceutically acceptable carrier.

The invention also provides:
An isolated nucleic acid molecule encoding the antigen binding protein as defined herein.

The invention also provides:
An isolated host cell transformed with the nucleic acid molecule as defined herein.

The invention also provides:
A method of treatment of Haemophilia A, comprising administering an antigen binding domain comprising an autoregulation element comprising a Thrombin cleavage site, or a composition comprising said autoregulation element, to an individual in need thereof.

The invention also provides:
A method of prevention of pro-thrombotic risks, comprising administering an antigen binding protein comprising an autoregulation element comprising a Thrombin cleavage site, or a composition comprising said autoregulation element, to an individual in need thereof.

The invention also provides:
A method of treatment of cancer, comprising administering an antigen binding protein comprising an autoregulation element comprising a Granzyme B cleavage site, or a composition comprising said autoregulation element, to an individual in need thereof.

The invention also provides:

A method of prevention of mode-of-action (MoA)-related toxicities associated with immunotherapies, comprising administering an antigen binding protein comprising an autoregulation element comprising a Granzyme B cleavage site, or a composition comprising said autoregulation element, to an individual in need thereof.

### BRIEF DESCRIPTION OF THE SEQUENCE LISTING

SEQ ID NO: 1: Thrombin cleavage recognition sequence consensus sequence
SEQ ID NO: 2: Thrombin cleavage recognition sequence P1′ G to L
SEQ ID NO: 3: Thrombin cleavage recognition sequence P1′ G to D
SEQ ID NO: 4: Thrombin cleavage recognition sequence from human coagulation Factor V (FV)
SEQ ID NO: 5: Granzyme B cleavage recognition sequence GZMB-1, from mouse BID protein
SEQ ID NO: 6: Granzyme B cleavage recognition sequence GZMB-2, from mouse BID protein
SEQ ID NO: 7: Granzyme B cleavage recognition sequence GZMB-3, consensus sequence
SEQ ID NO: 8: Granzyme B cleavage recognition sequence GZMB-4, P10-P10′ sequence of GZMB-2
SEQ ID NO: 9: Granzyme B cleavage recognition sequence GZMB-5, P4 I to V
SEQ ID NO: 10: Granzyme B cleavage recognition sequence GZMB-6, P4 I to L
SEQ ID NO: 11: Granzyme B cleavage recognition sequence GZMB-7, P2 P to G
SEQ ID NO: 12: Granzyme B cleavage recognition sequence GZMB-8, P2 P to A
SEQ ID NO: 13: Granzyme B cleavage recognition sequence GZMB-9, P1'S to A
SEQ ID NO: 14: Granzyme B cleavage recognition sequence GZMB-10, P2′ L to E and P4′ E to Q
SEQ ID NO: 15: Granzyme B cleavage recognition sequence GZMB-11, P2′ L to E and P4′ E to V
SEQ ID NO: 16: Granzyme B cleavage recognition sequence GZMB-12, P1 D to N
SEQ ID NO: 17: Granzyme B cleavage recognition sequence GZMB-13, P1 D to E
SEQ ID NO: 18: Granzyme B cleavage recognition sequence GZMB-14, P1 D to Q
SEQ ID NO: 19: ROR1-binding antigen binding domain LCDR 1
SEQ ID NO: 20: ROR1-binding antigen binding domain LCDR 2
SEQ ID NO: 21: ROR1-binding antigen binding domain LCDR 3
SEQ ID NO: 22: ROR1-binding antigen binding domain HCDR 1
SEQ ID NO: 23: ROR1-binding antigen binding domain HCDR 2
SEQ ID NO: 24: ROR1-binding antigen binding domain consensus HCDR 3
SEQ ID NO: 25: ROR1-binding antigen binding domain HCDR 3, Clone F Heavy Chain CDR3
SEQ ID NO: 26: ROR1-binding antigen binding domain HCDR 3, Humanised 1 Heavy Chain CDR3
SEQ ID NO: 27: ROR1-binding antigen binding domain HCDR 3, Humanised 2 and 5 Heavy Chain CDR3
SEQ ID NO: 28: ROR1-binding antigen binding domain HCDR 3, Humanised 3 and 4 Heavy Chain CDR3
SEQ ID NO: 29: ROR1-binding antigen binding domain, Clone F light chain variable region
SEQ ID NO: 30: ROR1-binding antigen binding domain, Humanised 1 light chain variable region
SEQ ID NO: 31: ROR1-binding antigen binding domain, Humanised 2 light chain variable region
SEQ ID NO: 32: ROR1-binding antigen binding domain, Humanised 3 light chain variable region
SEQ ID NO: 33: ROR1-binding antigen binding domain, Humanised 4 light chain variable region
SEQ ID NO: 34: ROR1-binding antigen binding domain, Humanised 5 light chain variable region
SEQ ID NO: 35: ROR1-binding antigen binding domain, Clone F heavy chain variable region
SEQ ID NO: 36: ROR1-binding antigen binding domain, Humanized 1 heavy chain variable region
SEQ ID NO: 37: ROR1-binding antigen binding domain, Humanized 2 heavy chain variable region
SEQ ID NO: 38: ROR1-binding antigen binding domain, Humanized 3 heavy chain variable region
SEQ ID NO: 39: ROR1-binding antigen binding domain, Humanized 4 heavy chain variable region
SEQ ID NO: 40: ROR1-binding antigen binding domain, Humanized 5 heavy chain variable region
SEQ ID NO: 41: CD3-binding antigen binding domain, mouse light chain variable region
SEQ ID NO: 42: CD3-binding antigen binding domain, Humanised 1 light chain variable region
SEQ ID NO: 43: CD3-binding antigen binding domain, Humanised 2 light chain variable region
SEQ ID NO: 44: CD3-binding antigen binding domain, Humanised 3 light chain variable region
SEQ ID NO: 45: CD3-binding antigen binding domain, Humanised 4 light chain variable region
SEQ ID NO: 46: CD3-binding antigen binding domain, Humanised 5 light chain variable region
SEQ ID NO: 47: CD3-binding antigen binding domain, mouse heavy chain variable region
SEQ ID NO: 48: CD3-binding antigen binding domain, Humanised 1 heavy chain variable region
SEQ ID NO: 49: CD3-binding antigen binding domain, Humanised 2 heavy chain variable region
SEQ ID NO: 50: CD3-binding antigen binding domain, Humanised 3 heavy chain variable region
SEQ ID NO: 51: CD3-binding antigen binding domain, Humanised 4 heavy chain variable region
SEQ ID NO: 52: CD3-binding antigen binding domain, Humanised 5 heavy chain variable region
SEQ ID NO: 53: CD3-binding antigen binding domain LCDR 1
SEQ ID NO: 54: CD3-binding antigen binding domain LCDR 2
SEQ ID NO: 55: CD3-binding antigen binding domain LCDR 3
SEQ ID NO: 56: CD3-binding antigen binding domain HCDR 1
SEQ ID NO: 57: CD3-binding antigen binding domain HCDR 2
SEQ ID NO: 58: CD3-binding antigen binding domain HCDR 3
SEQ ID NO: 59: linker sequence
SEQ ID NO: 60: linker sequence
SEQ ID NO: 61: ROR1×CD3 bispecific antibody amino acid sequence
SEQ ID NO: 62: ROR1×CD3 bispecific antibody amino acid sequence with an N-terminal hexa-histidine tag (NVG-111)
SEQ ID NO: 63: common LCDR 1 of FIX-binding and FX-binding antigen binding domains
SEQ ID NO: 64: common LCDR 2 of FIX-binding and FX-binding antigen binding domains
SEQ ID NO: 65: common LCDR 3 of FIX-binding and FX-binding antigen binding domains
SEQ ID NO: 66: FIX-binding antigen binding domain HCDR 1
SEQ ID NO: 67: FIX-binding antigen binding domain HCDR 2
SEQ ID NO: 68: FIX-binding antigen binding domain HCDR 3
SEQ ID NO: 69: FX-binding antigen binding domain HCDR 1
SEQ ID NO: 70: FX-binding antigen binding domain HCDR 2
SEQ ID NO: 71: FX-binding antigen binding domain HCDR 3
SEQ ID NO: 72: common light chain variable region of FIX-binding and FX-binding antigen binding domains
SEQ ID NO: 73: heavy chain variable region of FIX-binding antigen binding domain
SEQ ID NO: 74: heavy chain variable region of FX-binding antigen binding domain
SEQ ID NO: 75: hinge region
SEQ ID NO: 76: hinge region plus SEQ ID NO: 1
SEQ ID NO: 77: linker sequence
SEQ ID NO: 78: linker sequence
SEQ ID NO: 79: linker sequence
SEQ ID NO: 80: linker sequence
SEQ ID NO: 81: linker sequence
SEQ ID NO: 82: hinge region of CAR embodiment
SEQ ID NO: 83: partially deleted hinge region of CAR embodiment plus SEQ ID NO: 7
SEQ ID NO: 84: hinge region of CAR embodiment plus SEQ ID NO: 7

### BRIEF DESCRIPTION OF THE FIGURES

FIG. 1: Schematic representation of the mode-of-action for autoregulation (AR) exemplified with therapeutic antibodies.

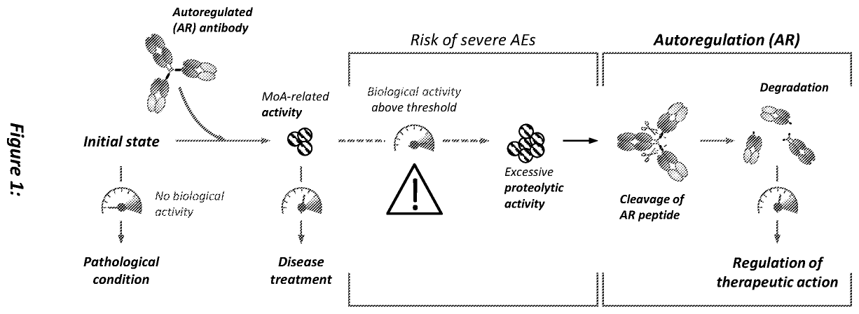

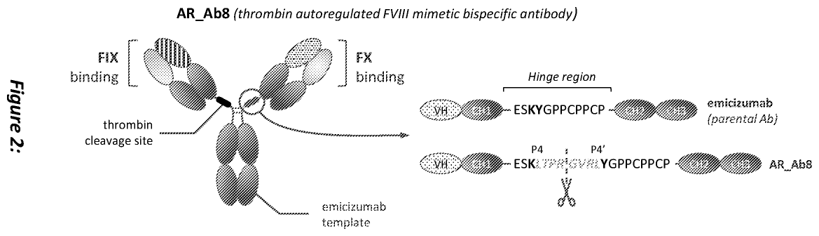

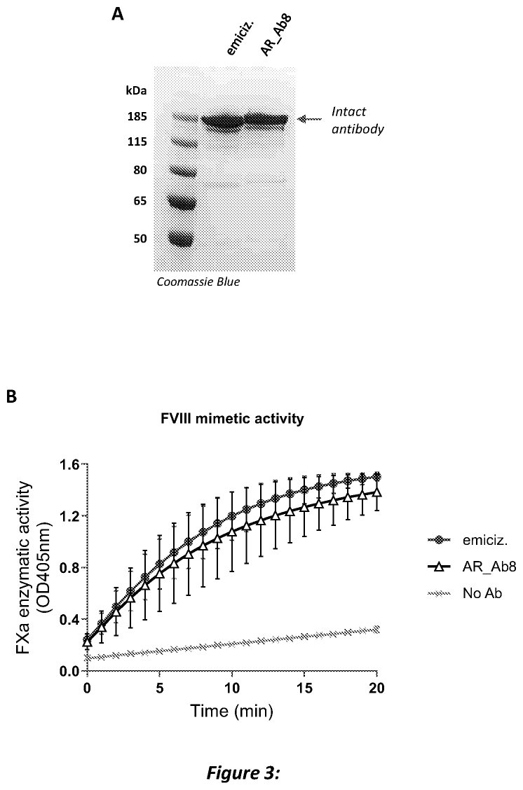

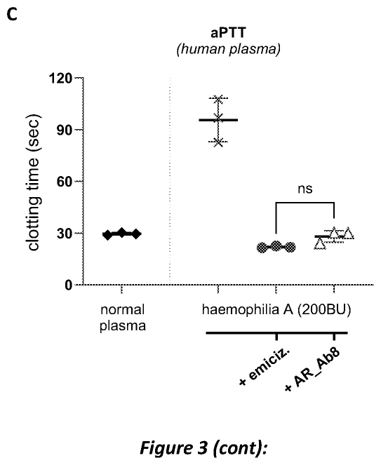

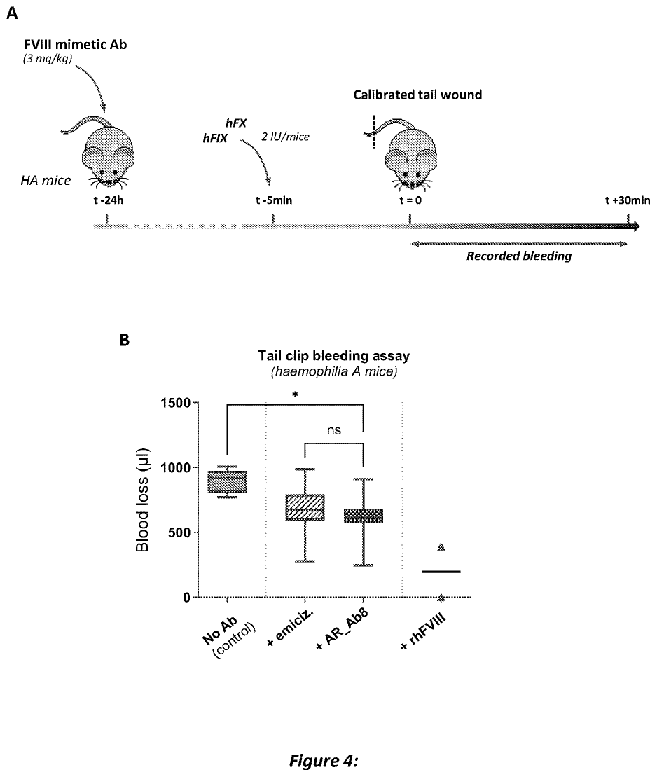

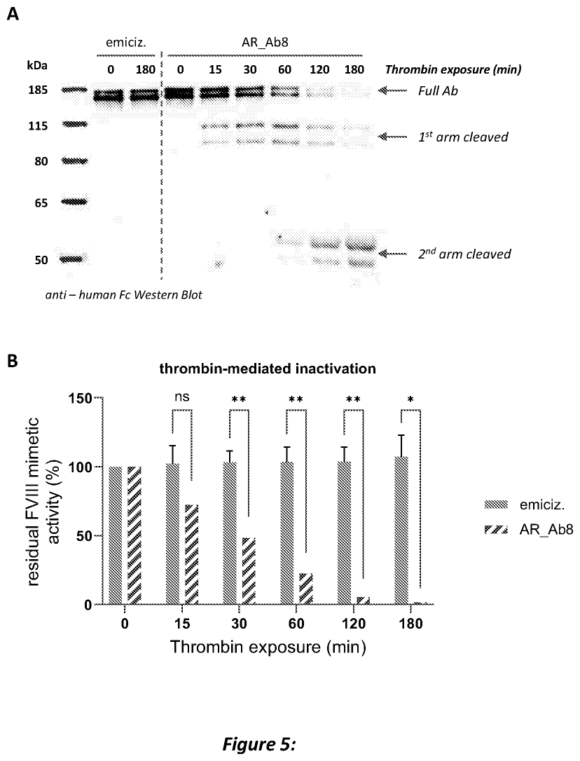

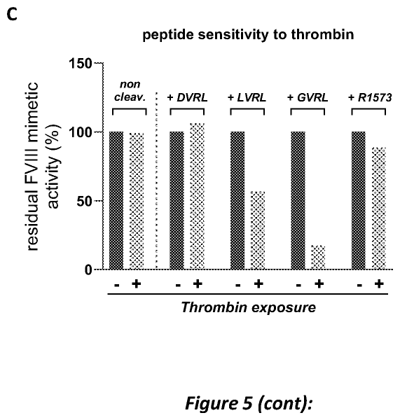

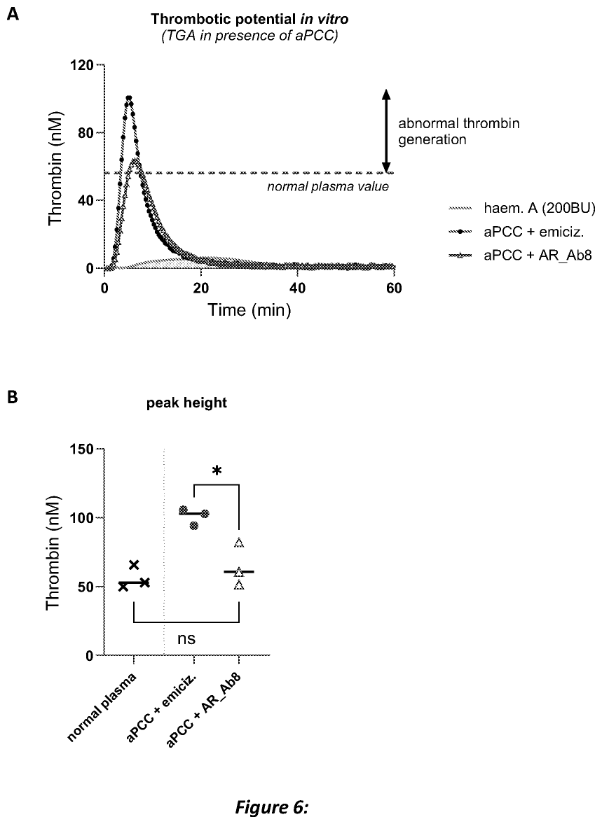

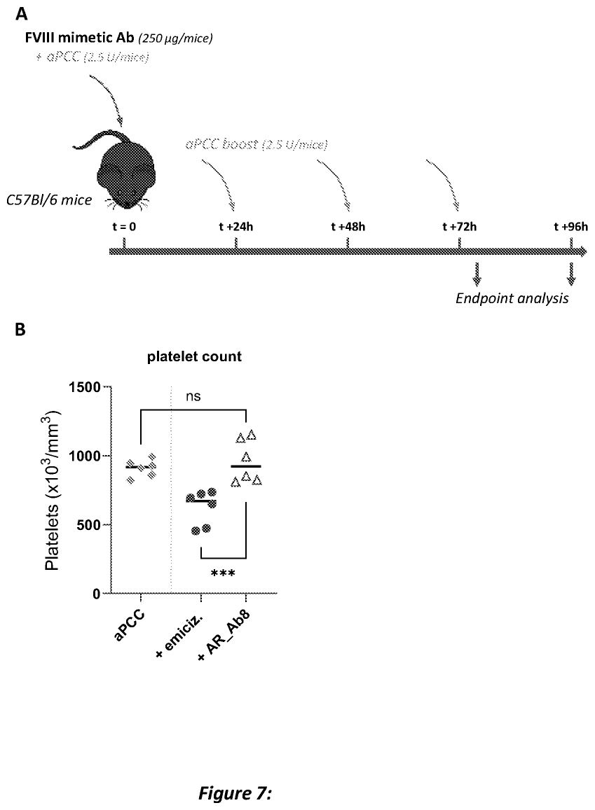

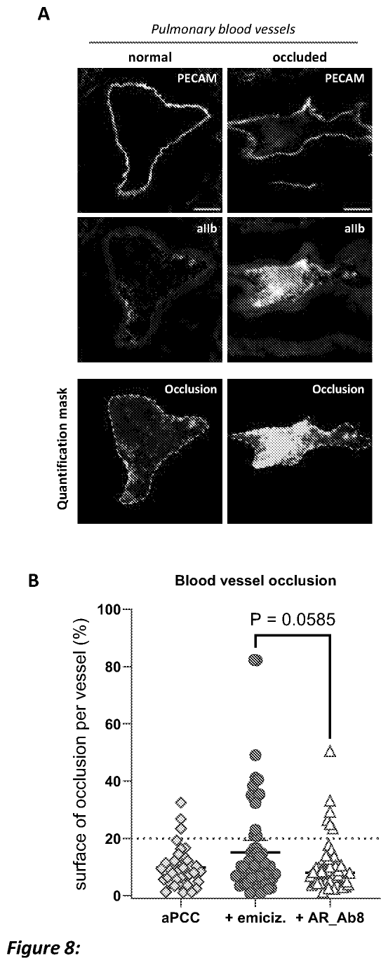

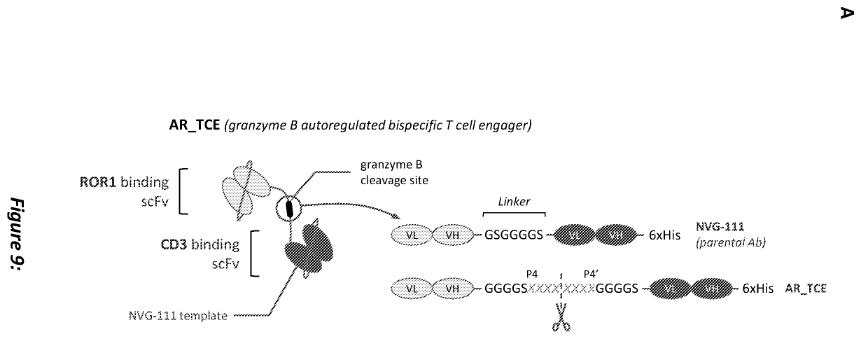

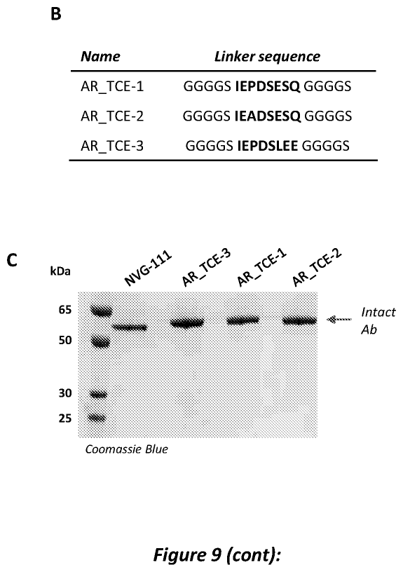

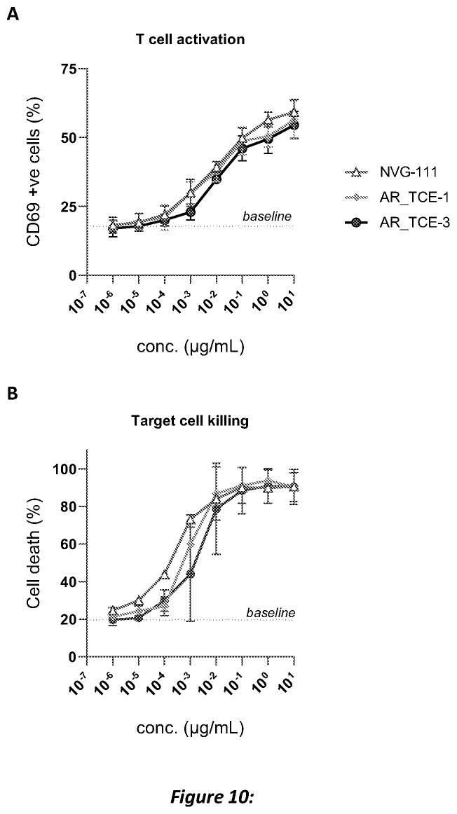

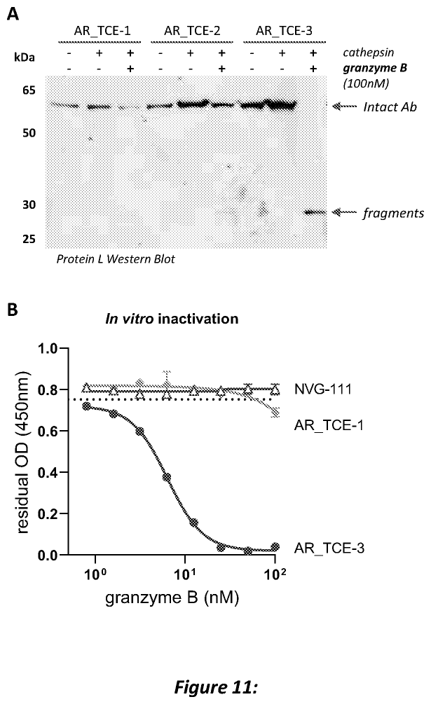

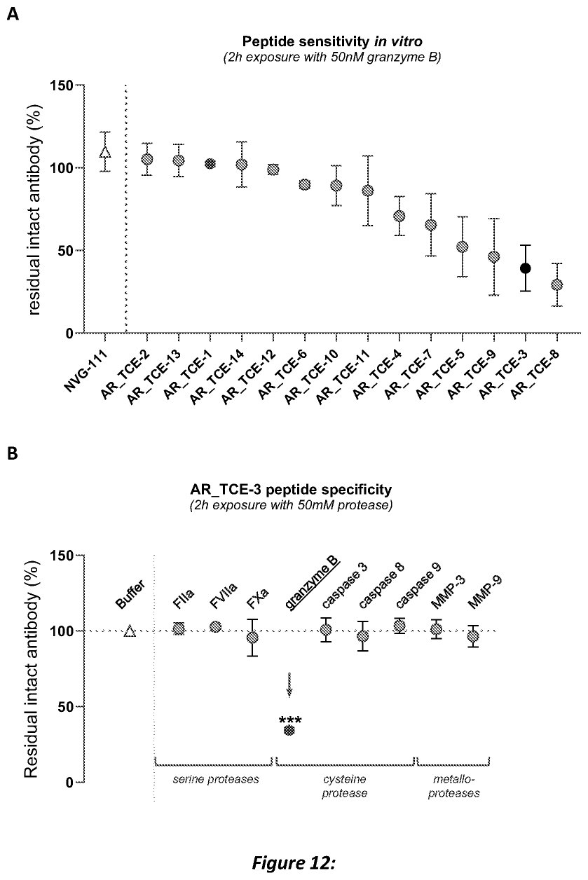

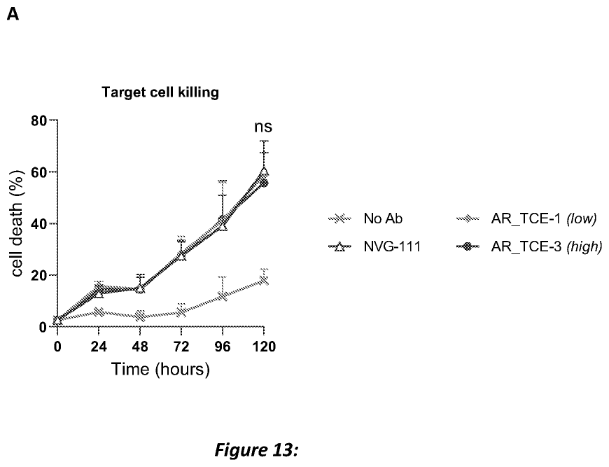

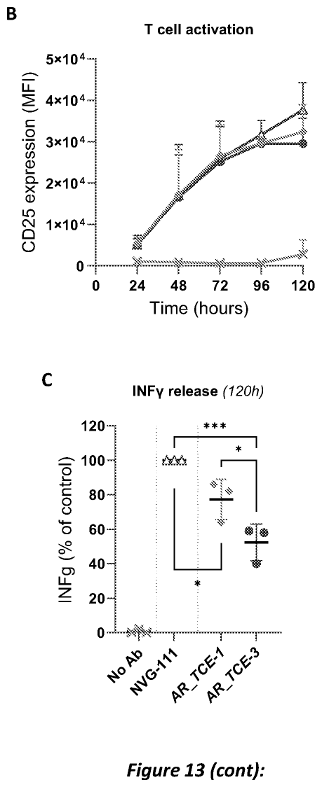

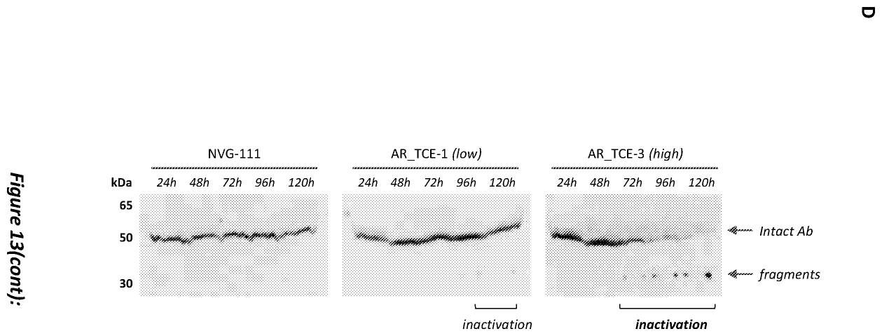

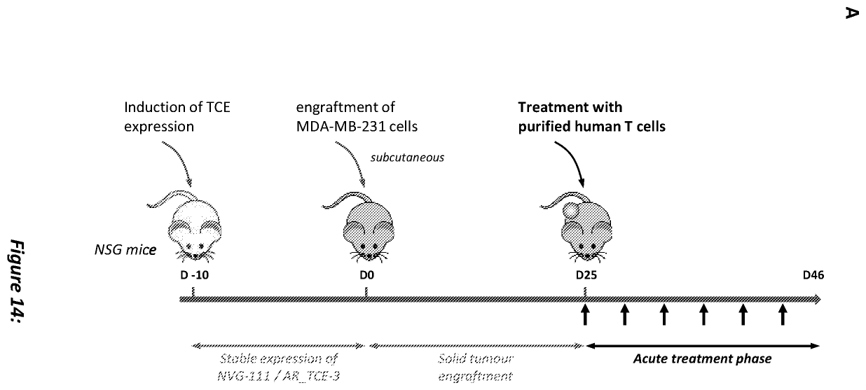

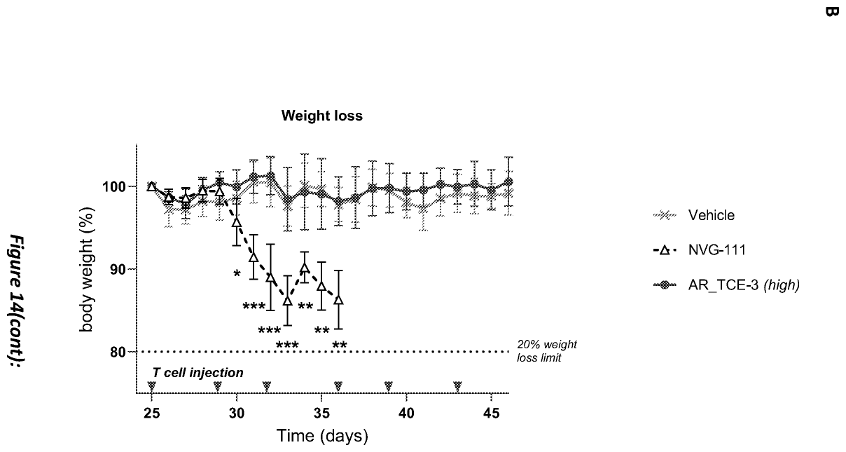

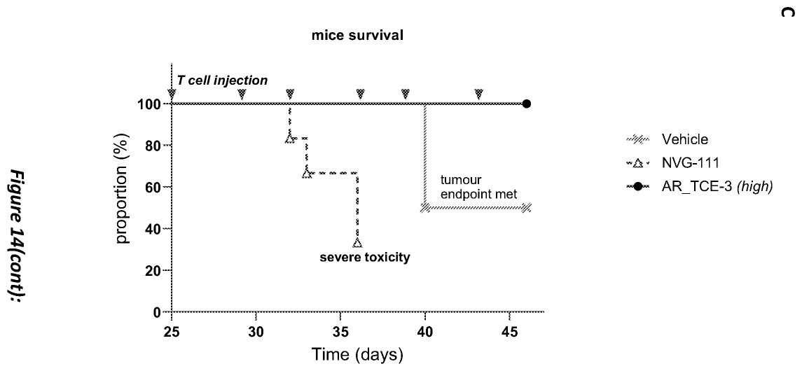

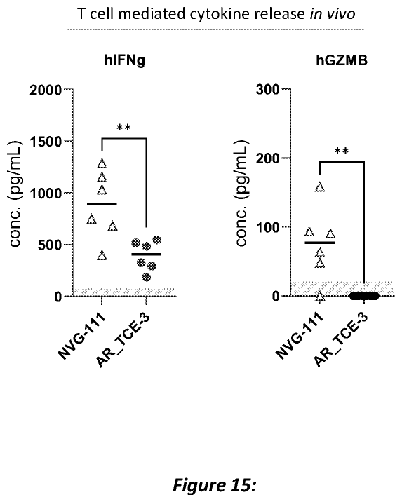

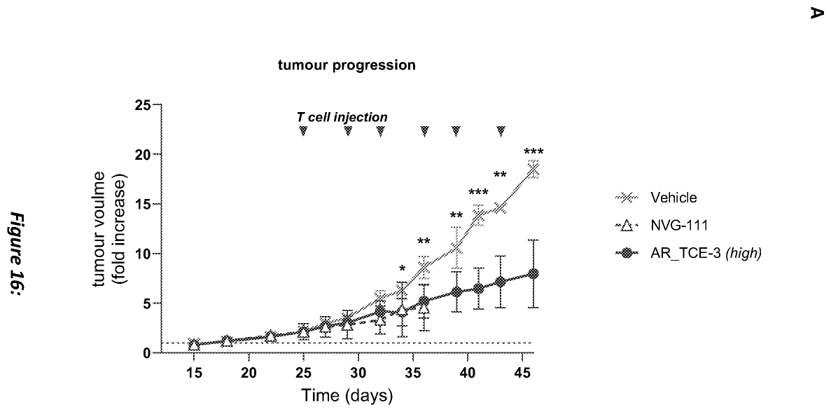

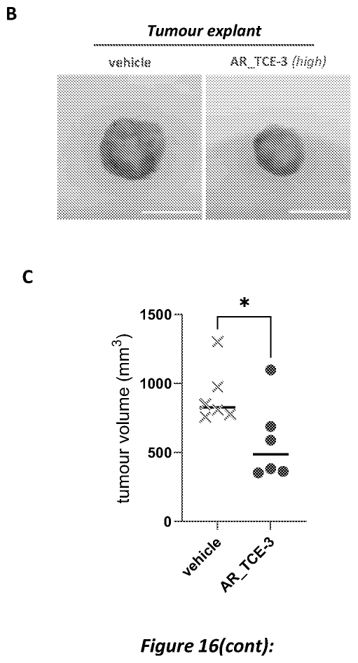

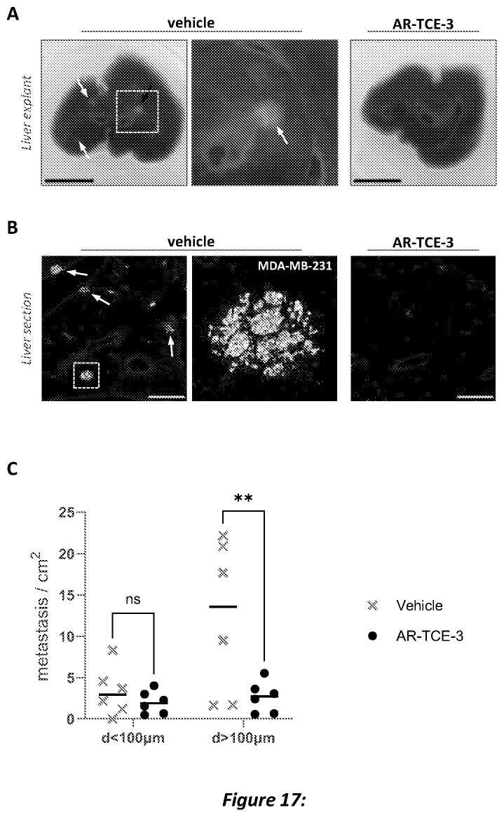

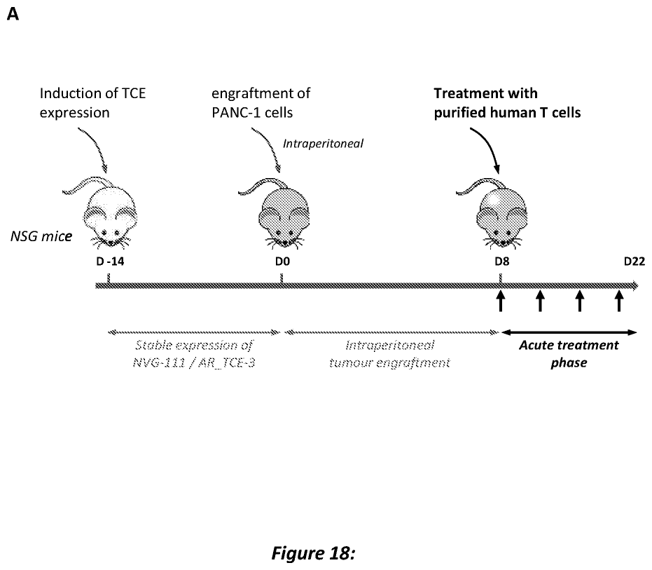

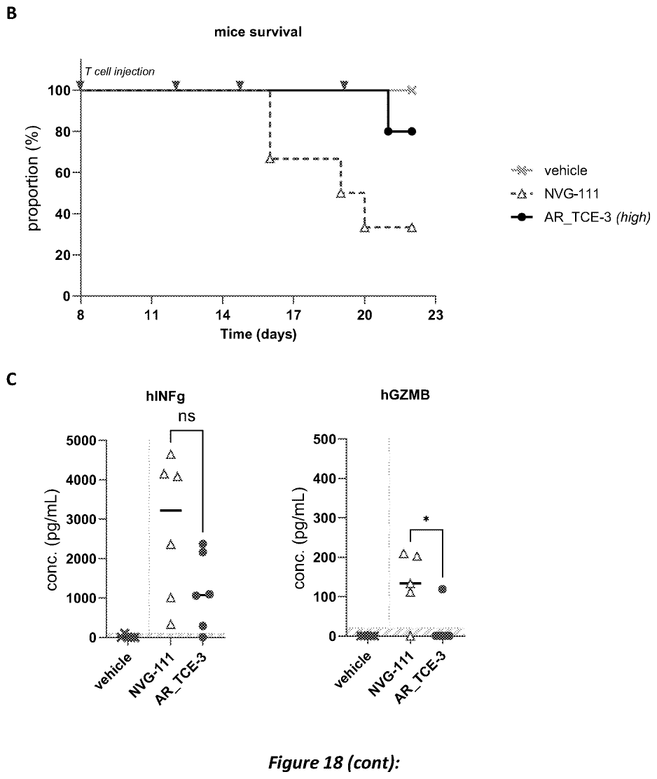

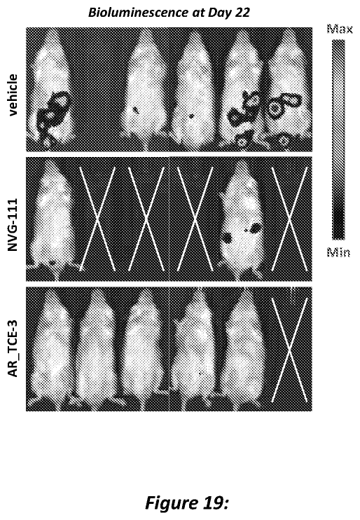

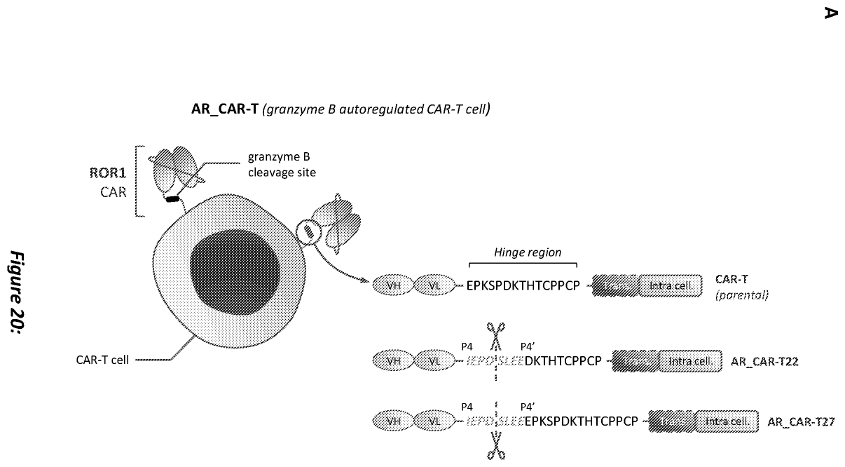

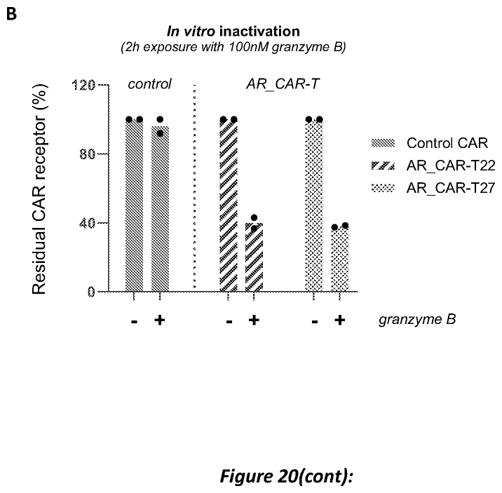

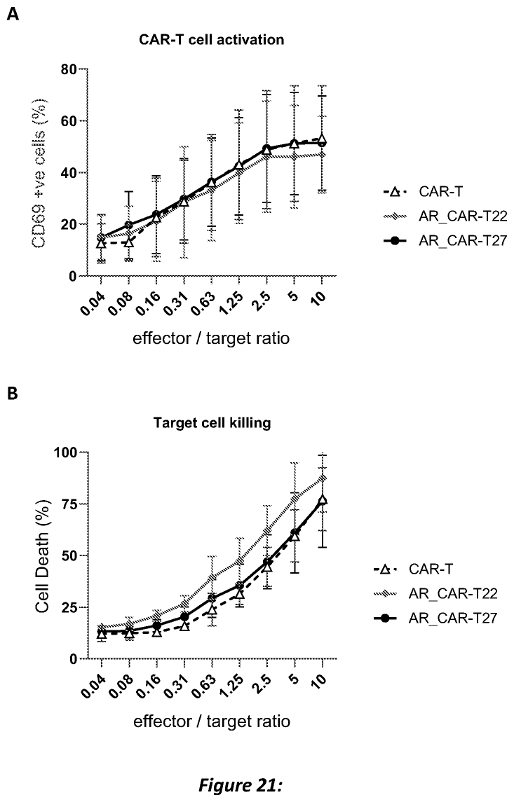

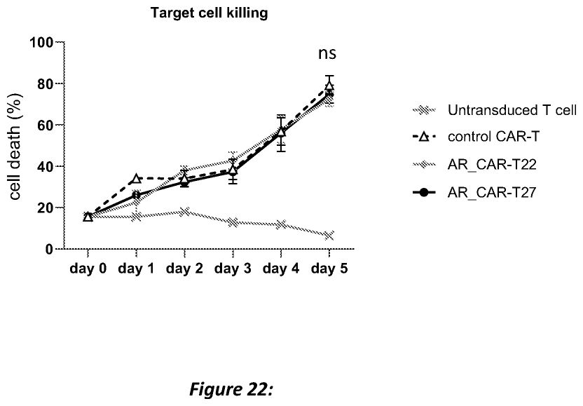

The initial pathological state is characterised by an absence of biological activity. In the context of a therapeutic intervention, the AR antibody (or therapeutic agent) generates the biological activity required for an efficient therapeutic action. In a situation of excessive biological activity leading to adverse events, a product with proteolytic activity resulting from the mode-of-action of the AR antibody is used to trigger autoregulation. Elevated activity of this enzymatic product will cleave specific sensitive-peptides inserted into the structure of the AR antibody (or therapeutic agent), thus leading to a progressive degradation of the molecule. The reduced concentration of the AR antibody will therefore reduce its biological activity and regulate the therapeutic efficacy to prevent the occurrence of adverse events.

FIG. 2: Design of an autoregulated bispecific antibody with FVIII mimetic activity for haemophilia A.

AR_Ab8, a prototype of autoregulated bispecific therapeutic antibody for the treatment of haemophilia A was designed using the core sequence of emicizumab, a full-size humanised IgG4 antibody targeting coagulation Factor IX (FIX) and Factor X (FX). A thrombin-sensitive cleavable peptide composed of 8 residues (P4 to P4′ sequence) was inserted in the hinge region of both heavy chains of the parental antibody.

FIG. 3: In vitro characterisation of the FVIII mimetic potential.

(A) SDS-Page gel stained with Coomassie blue illustrating the migration profile in non-reducing condition of purified emicizumab and AR_Ab8, showing the absence of premature degradation. (B) The FVIII mimetic activity of both emicizumab and AR_Ab8 (2 nM) were tested in a chromogenic assay measuring the conversion of FX into FXa by FIXa. Dots indicate the mean OD value±SD of three replicates. Control condition was performed in absence of bsAb. (C) In vitro procoagulant potential was measured in human plasma. A pool of citrated normal human plasma was spiked with 200 BU of anti-FVIII antibody to neutralise endogenous FVIII. This induced haemophilia A plasma was then treated with 350 nM of emicizumab or AR_Ab8 and activated Partial Thromboplastin Time (aPTT) was measured. Symbols represent individual clotting times values and lines indicate the mean±SD of three replicates. ns means non-significant in a one-way ANOVA test with Sidak's multiple comparisons.

FIG. 4: Uncompromised therapeutic efficacy of AR_Ab8 in haemophilia A mice. (A) The procoagulant efficacy of AR_Ab8 was tested in vivo. FVIIIKO mice received an IV injection with 3 mg/kg of emicizumab or AR_Ab8. 24 h later the mice were injected with a bolus dose of human FIX and FX and a tail clip assay was then performed 5 min after the second injection. Control animals received 2 U/mice of human FVIII. (B) Total blood loss results are presented as Wisker plots with the median (line), 25^{th} and 75^{th} percentile (upper and lower boxes) and min/max (bars) of 6 to 11 mice. Symbols indicate individual values. ns and * means non-significant and p<0.05 respectively in a one-way ANOVA test with Sidak's multiple comparisons.

FIG. 5: Thrombin-mediated degradation of AR_Ab8 in vitro 2 nM of either AR_Ab8 or emicizumab was incubated with 2 U/mL of α-thrombin to evaluate the cleavage kinetic of the thrombin sensitive peptide. The enzymatic cleavage reaction was stopped with the addition of 500 nM PPACK at different time-point up to 180 min. (A) Reaction samples were migrated on a SDS-Page gel in non-reducing conditions and immunoblotted with an anti-human Fc antibody to visualize the degradation of AR_Ab8. (B) The residual FVIII mimetic activity of both antibodies was then measured in the FXa chromogenic assay. Results were normalised and expressed as percentage of emicizumab for each time-point. Bars represent the mean±SD of three replicates. ns, * and ** means non-significant, p<0.05 and p<0.01 in a two-way ANOVA test with Sidak's multiple comparisons. (C) The sensitivity of various thrombin cleavable peptides was tested in a bispecific tandem_scFv format. 2 nM of the different tandem-scFvs were exposed to 2 U/mL of α-thrombin for 20 min, and the residual FVIII mimetic was measured in the FXa chromogenic assay. Results were normalised to the control condition (without thrombin exposure) and bars represent the percentage of residual activity. A negative control (non-cleav.) was performed with a non-autoregulated construct.

FIG. 6: Thrombin-mediated AR reduces abnormal thrombin generation in vitro In vitro thrombogenic profile of AR_Ab8 was investigated using a model of thrombin generation in presence of activated Prothrombin Complex Concentrate (aPCC) which potentialized abnormal thrombin generation with FVIII mimetic antibodies. A pool of citrated normal human plasma was spiked with 200 BU of anti-FVIII antibody to neutralise endogenous FVIII and further spiked with 0.5 U/mL of aPCC combined with 600 nM. Thrombin generation was then trigger with the Low Tissue Factor solution and measured in the Calibrated Automated Thrombogram (CAT) method (A) Dotted line shows the mean value of the maximum of thrombin generated in normal plasma and curves above the dotted line show abnormal thrombin generation in the mean of three replicates. Peak height (maximal thrombin generation) was extracted from the thrombogram and plotted as individual values (symbols) and mean of three replicates (B). ns and * means non-significant and p<0.05 respectively in a one-way ANOVA test with Sidak's multiple comparisons.

FIG. 7: Thrombin-mediated AR reduces the thrombotic profile of FVIII mimetic antibody in mice

In vivo thrombogenic profile of bispecific antibodies with FVIII mimetic activity was evaluated in a mice model co-treated with aPCC, which potentialize the thrombotic potential of emicizumab. (A) C57Bl/6 male mice first received a bolus intravenous injection of aPCC (2.5 U) and emicizumab or AR_Ab8 (250 ug) followed by 3 booster injection of aPCC 24 h apart. At t+96 h the thrombotic outcome was evaluated. (B) Plasma samples were collected on EDTA anticoagulant at termination of the experiment (96 h) and the platelet count was measured. Symbols are individual animals and horizontal bar represent the median distribution of each experimental condition. ns means not-significant and *** means p<0.001 in an unpaired t-test.

FIG. 8: AR_Ab8 reduces the formation of pulmonary thrombi in mice

Lungs of animals treated with aPCC and emicizumab or AR_Ab8 were dissected, fixed in formalin and sections were mounted for histological evaluation. (A) Immunofluorescent staining against PECAM (green) and platelet specific integrin αIIb (purple) was performed on lung sections. PECAM staining was used to delimit the blood vessel surface (dotted line) and the αIIb staining was transformed in a binary occlusion mask. Objective 20×, scale bars measure 50 um. (B) Blood vessels occlusion was quantified in a blinded manner and was calculated as the percentage of occluded mask within the total blood vessel surface. Symbols represent occlusion values of each blood vessels measured from eight different animals per groups in two independent experiments. Horizontal bar represent the median distribution of each experimental condition and p value was calculated in an unpaired t-test.

FIG. 9: Design of an autoregulated bispecific T cell engager targeting ROR1. Autoregulated bispecific T cell engagers (AR_TCE) were designed using the core sequence of NVG-111, a CD3×ROR1 bispecific tandem scFvs antibody with indications for the treatment of ROR1 positive solid and haematological malignancies. (A) The linker region connecting the two scFvs of the parental antibody was replaced with a granzyme B sensitive cleavable peptide composed of 8 residues (P4 to P4′ sequence) and flanked with 2×G₄S motifs. (B) Three variants of the AR_TCE were developed with different granzyme B cleavable P4 to P4′ sequences. (C) All variants were expressed in Expi-293 cells and show similar migration profile to the parental antibody NVG-111 on a SDS-Page in reducing conditions followed by Coomassie blue staining.

FIG. 10: T cell engagement capacity of AR_TCEs.

The potential for T cell engagement of AR_TCE variants was tested in a short term killing assay model. Increasing concentrations of NVG-111 or AR_TCEs were incubated for 48 h in a coculture model with ROR1 positive Jeko-1 and purified human T cells from healthy donors with a 5:1 effector to target ratio. (A) The level of T cell activation was measured by direct staining of CD69 in flow cytometry and expressed as the percentage of positive cells within the T cell population. (B) In parallel, The percentage of cell death was measured in the target cell population using incorporation of a viability dye. Symbols are the mean±SD of three replicates. The dotted lines show basal cell death and CD69 expression in absence of antibody.

FIG. 11: Granzyme B dose-dependent mediated inactivation of AR_TCEs in vitro

The ability of AR_TCE variants to be cleaved and inactivated by granzyme B was tested in vitro. Recombinant granzyme B was first activated using cathepsin C. Activated granzyme B was then incubated at 100 nM final with 1 ug/mL of NVG-111 or the AR_TCE variants for 2 h at 37° C. Controls were done with buffer only or cathepsin C alone. (A) Samples were migrated on an SDS-Page gel in reducing conditions and immunoblotted using protein L to detect intact and fragment antibodies. NVG-111 or the AR_TCE variants (1 ug/mL) were then incubated at 37° C. with increasing concentrations of activated granzyme B. The reaction was then stopped at 2 h by the addition of a broad-spectrum protease inhibitor. (B) The residual intact antibodies were detected using a dedicated ELISA assay. Symbols are the mean±SD of two replicates and curves fit a 4-parameters variable slope model using Prism software.

FIG. 12: In vitro characterisation of sensitivity and specificity for AR_TCEs

(A) An extended range of AR_TCEs with various AR peptide sequences was incubated with a single concentration of granzyme B (50 nM) for 2 h and the reaction was stopped by addition of a broad-spectrum protease inhibitor. (B) AR_TCE-3 was then exposed for 2 h to a broad panel of proteases at a similar concentration. The residual intact antibodies were detected by ELISA. Residual antibody was normalised to the non-treated counterpart and expressed as percentage of residual intact antibody. In (B) he results were ranked from highest to lowest percentage. Symbols are the mean±SD of 3 replicates. *** means p<0.001 in a one-way ANOVA test with Sidak's multiple comparisons.

FIG. 13: Granzyme B mediated AR in reduces cytokine release in vitro

The efficacy and safety profile of 2 AR_TCE variants with low (AR_TCE-1) and high (AR_TCE-3) cleavage rate was evaluated in a long-term coculture assay. NVG-111 or the AR_TCE variants were incubated at 1 μg/mL with ROR1 positive Jeko-1 and purified human T cells from healthy donors using an excess of target cells (1:10 effector to target ratio), for up to 120 h. (A) The percentage of cell death was measured every 24 h by flow cytometry in target cells using incorporation of a viability dye. (B) The level of T cell activation was measured by direct staining of CD25 and expressed as mean fluorescence intensity (MFI) the CD3 positive population. Symbols are the mean±SD of 3 replicates. (C) Levels of human interferon gamma (IFNγ) release by activating T cells was measured in the culture supernatant at 120 h. Symbols are individual values and horizontal bars are the mean±SD of three replicates. * and *** mean p<0.05 and p<0.001 respectively in a one-way ANOVA test with Sidak's multiple comparisons. (D) Supernatant samples were migrated on a SDS-Page gel and immunoblotted using an anti His-Tag antibody to detect cleavage and inactivation of the AR_TCE constructs.

FIG. 14: Granzyme B mediated AR increases survival in a mouse xenograft model of TNBC.

The safety profile of AR was evaluated in a murine model of TCE-induced cytokine-mediated toxicity. (A) Immunocompromised NOD.SCID Gamma mice were first induced or not (vehicle) with stable expression of either NVG-111 or AR_TCE-3. At day 0 all animals were engrafted with a subcutaneous injection of 2·10⁶ human ROR1⁺ triple negative breast cancer (TNBC) MDA-MB-231 cells and the tumour was allowed to engraft for 25 days. Mice then received 6 cycles of purified human T cells. Animals were carefully monitored during the treatment phase and culled when ethically required or at day 46 upon completion of the study. (B) The total body weight of individual animals was recorded every 24 h and presented as percentage of the measured weight on the day of the 1^{st} T cell injection (day 25). Symbols and connecting curves represent the mean±SD of 6 animals per group. Arrows mark the days of T cell injection. *, ** and *** mean p<0.05, p<0.01 and p<0.001 respectively in a one-way ANOVA test with Sidak's multiple comparisons. (C) Animals meeting one of the study endpoint (severe weight loss, acute toxicity or tumour ulceration) were either culled or taken of the study (treatment with T cell stopped). Results are presented as proportion of surviving animals and plotted as a Kaplan-Meier curve.

FIG. 15: Granzyme B mediated AR reduces TCE-induced toxicity and cytokine release in a model of TNBC.

In mice engrafted with solid tumour of triple negative breast cancer and treated with either NVG-111 or AR_TCE-3 in combination with human T cells, plasma samples were prepared at the time of culling. Circulating levels of T cell derived cytokine, including human IFNγ and granzyme B were measured by ELISAs. Symbols are individual values and horizontal bars represent the mean distribution for each condition. Dashed area shows the lower limit of detection and N.D. means not detectable. ns and ** means non-significant and p<0.01 respectively in an unpaired t-test.

FIG. 16: AR_TCE shows uncompromised therapeutic efficacy in a TNBC model

(A) The efficacy of AR_TCE was evaluated on tumour progression. Tumour size was measured on 2 axis using a micrometric calliper and volume was calculated using the sphere formula. Volumes calculated at day 15 and 18 were pooled to reduce variability and the subsequent measures were normalised to this value. Results are presented as fold-increase from the normalised baseline. Symbols and connecting curves represent the mean±SD of 6 animals per group. Arrows mark the days of T cell injection. *, ** and *** mean p<0.05, p<0.01 and p<0.001 respectively in a one-way ANOVA test with Sidak's multiple comparisons. (B) Tumours were explanted upon culling of the animals and photographed with bars representing 10 mm. (C) Explant volumes were measured from the images and presented as mean volume. Symbols are individual values and * means p<0.05 in an unpaired statistical t-test.

FIG. 17: AR_TCE reduces metastatic progression in a TNBC model

Impact of the extended therapeutic index of TCEs in presence of AR was evaluated on metastatic progression of the TNBC model. (A) The liver of vehicle and AR_TCE-3 treated animals was explanted upon termination of the study and photographed with bars representing 10 mm. (B) Immunofluorescent staining against the MDA-MB-231 cells was performed on liver section to evaluate the metastatic infiltration in the tissue and images were acquired in widefield microscopy with bars representing 1 mm. Arrows indicate large cancer cell clusters and dotted squares delimits the magnified area. (C) The density of metastatic nodules was quantified from the fluorescent images and sorted by nodule diameter (d)< or > to 100 m. Symbols represent individual animals and bars are mean value for each condition. ns means nonsignificant and **P<0.01 in a 2-way ANOVA test with Sidak's multiple comparisons

FIG. 18: Granzyme B mediated AR reduces TCE induced cytokine release and increase survival in an intraperitoneal model of pancreatic cancer.

The safety profile of AR was evaluated in a murine model of ROR1⁺ pancreatic tumour engrafted in the peritoneal cavity. (A) Immunocompromised NOD.SCID Gamma mice were first induced or not (vehicle) with stable expression of either NVG-111 or AR_TCE-3. At day 0 all animals received a single intraperitoneal injection with 2.10⁶ human pancreatic cancer PANC-1 cells, and were allowed to recover for 8 days. Mice then received 4 cycles of purified human T cells. (B) Animals were monitored carefully during the treatment phase and ethically culled when meeting one of the study endpoint (severe weight loss, acute toxicity). Survival results are presented as proportion of animals and plotted in a Kaplan-Meier curve. (C) Plasma samples were prepared from all animals 24 h after the 2^{nd} T cell injection and cytokine levels were measured by ELISAs. Symbols are individual values and horizontal bars represent the mean distribution for each condition. Dashed area shows the lower limit of detection and N.D. means not detectable. ns and * means non-significant and p<0.05 respectively in an unpaired t-test.

FIG. 19: AR_TCE reduces the tumour burden in a model of intraperitoneal pancreatic cancer.

In mice engrafted with intraperitoneal pancreatic cancer and treated with either NVG-111 or AR_TCE-3 in combination with human T cells, tumour progression was monitored using by bioluminescent imaging on an IVIS imager. Mice received and intraperitoneal injection of D-luciferin to detect the Luciferase-positive PANC-1 and were imaged 15 min later. Colour range indicates tumour bioluminescence intensity and white crosses marks mice ethically culled before day 22.

FIG. 20: Design of autoregulated CAR-T cells targeting ROR1.

Autoregulated CAR-T cells (AR_CAR-T) were designed using the template of a ROR1 CAR-T cell. (A) The upper region of the hinge connecting the ROR1 targeting scFv and the CD8a transmembrane domain of the CAR receptor was modified to introduce the P4 to P4′ sequence of granzyme B sensitive cleavable peptide. (B) The ability of the modified AR_CAR receptor to be cleaved of the CAR-T cells by granzyme B was tested in vitro. Control CAR-T or the AR_CAR-T cell variants were incubated with 100 nM of granzyme B for 2 h at 37° C. The presence of residual CAR receptor at the surface of the cells was detected with fluorescent protein L in flow cytometry. Results were normalised to the non-treated counterpart and expressed as percentage of residual CAR receptor. Histograms represent the average and symbols are individual values.

FIG. 21: Activation and target engagement capacity of AR_CAR-T cells variants.

Target engagement and activation of AR_CAR-T cells were tested in a short-term killing assay model where ROR1 positive Jeko-1 cells were cocultured with CAR-T cells at various effector to target ratios, for 48 h. (A) The level of activation in CAR-T cells was measured by direct staining of CD69 in flow cytometry and expressed as the percentage of positive cells. (B) In parallel, the percentage of target cell death was measured in the Jeko-1 population using incorporation of a viability dye. Symbols represent the mean±SD of 4 replicates.

FIG. 22: AR_CAR-T cells maintains uncompromised serial killing potential in vitro.

The Serial killing potential of AR_CAR-T cell variants was evaluated in a long-term coculture assay with an excess of target cells. Control CAR-T or the AR_CAR-T cells were cocultured for 5 days with ROR1 positive Jeko-1 at a 1:10 effector to target ratio. The percentage of target cell death was measured every 24 h by flow cytometry using incorporation of a viability dye. Symbols are the mean±SD of 4 replicates. ns means non-significant in a one-way ANOVA test with Sidak's multiple comparisons.

### DETAILED DESCRIPTION OF THE INVENTION

It is to be understood that different applications of the disclosed invention may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural references unless the content clearly dictates otherwise. Thus, for example, reference to “an antibody” includes “antibodies”, and the like.

All publications, patents and patent applications cited herein, whether supra or infra, are hereby incorporated by reference in their entirety.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Unless otherwise explained, all technical and scientific terms used herein have the same meaning as commonly understood by one of ordinary skill in the art to which this disclosure belongs.

In general, the term “comprising” is intended to mean including, but not limited to. For example, the phrase “A therapeutic agent comprising a polypeptide” should be interpreted to mean that the agent comprises at least one polypeptide, but the agent may also comprise further components.

In some embodiments of the invention, the word “comprising” is replaced with the phrase “consisting of”. The term “consisting of” is intended to be limiting. For example, the phrase “A therapeutic agent consisting of a polypeptide” should be interpreted to mean that the agent consists of a polypeptide and no further components.

In some embodiments of the invention, the word “comprising” is replaced with the phrase “consisting essentially of”. The term “consisting essentially of” means that specific further components can be present, namely those not materially affecting the essential characteristics of the subject matter. For example, the phrase “A therapeutic agent comprising essentially of a polypeptide” may also comprise other components such as linker sequences or labels in addition to the polypeptide.

The terms “self-regulation” and “autoregulation” are equivalent and are used interchangeably in the disclosure herein. The term “AR” is an abbreviation of “autoregulation”. “AR” is an equivalent term to “autoregulation” and “self-regulation”.

### Therapeutic Agent

The therapeutic agent of the invention is any substance that is useful for achieving a therapeutic end or result; for example, a substance or combination of substances useful for preventing or treating a disease or condition. Therapeutic agents include, but are not limited to, proteins, nucleic acid molecules, and other molecules of interest. In some embodiments, the therapeutic agent comprises a polypeptide, such as an antigen-binding protein. In some embodiments, the therapeutic agent consists essentially of a polypeptide, such as an antigen binding protein. In some embodiments, the therapeutic agent is a polypeptide, such as an antigen binding protein. The skilled artisan will understand that particular agents may be useful to achieve more than one result. Therapeutic agents act on a metabolic pathway associated with a disease or condition, in order to treat said disease or condition or prevent symptoms associated with said disease or condition. Treating can include alleviation of symptoms and includes the prevention of side effects resulting from adverse effects of such treatment.

A therapeutically effective amount of the therapeutic agent can be defined as a quantity of said agent, such as a disclosed antigen-binding protein, sufficient to achieve a desired effect in a subject being treated. For instance, this can be the amount necessary to inhibit tumour growth, or prevent haemorrhage. In several embodiments, a therapeutically effective amount is the amount necessary to reduce a symptom of the disease or condition. When administered to a subject, a dosage will generally be used that will achieve target tissue concentrations that has been shown to achieve a desired in vitro effect.

### Self-Regulation/Autoregulation Element

The self-regulation or autoregulation element of the invention is a small peptide cleavable by an endogenous factor. A small peptide can be defined as an amino acid sequence of at least 4 amino acids but no more than 15 amino acids in length, preferably at least 5, 6, 7 amino acids, more preferably at least 8 amino acids but no more than 15 amino acids in length. In a preferred embodiment, the self-regulation/autoregulation element is 7, 8, 9 or 10 amino acids in length. In an especially preferred embodiment, the self-regulation/autoregulation element is 8 amino acids in length.

The self-regulation element does not affect the activity of the therapeutic agent to a significant extent. That is, the therapeutic agent can function it its therapeutic range in the presence of the self-regulation element. Suitable tests for therapeutic activity are known to the person skilled in the art. The self-regulation element is positioned in an exposed position within the therapeutic agent, so that the self-regulation element is accessible to the endogenous factor.

In the therapeutic agent of the invention, the activity of the endogenous factor is modified as a consequence of the activity of the therapeutic agent, and cleavage of the self-regulation element by the endogenous factor results in separation of the therapeutic agent into non-functional fragments. Non-functional fragments can be defined as fragments that no longer have the required therapeutic activity, or fragments that are subject to degradation, or elimination, at a rate that would make them functionally inactive. Therapeutic activity can include binding to a target antigen. Binding kinetics can be investigated by standard laboratory techniques.

The endogenous factor as described herein is a factor present endogenously in the subject to be treated. The endogenous factor as described herein has proteolytic activity. In a preferred embodiment of the invention the endogenous factor is a protease. In a preferred embodiment of the invention the self-regulation element comprises a proteolytic cleavage site.

The self-regulation element of the invention can be defined according to the proteolytic cleavage recognition site below:
P4-P3-P2-P1-site of cleavage-P1′-P2′-P3′-P4′,
   wherein P denotes a single amino acid (aa).

The endogenous factor is one active in the same metabolic pathway as that acted on by the therapeutic agent of the invention. It is envisaged that suitable endogenous factors can be chosen upon consideration of the metabolic pathways of disease acted on by the therapeutic agent.

By way of illustration but not limitation, the following proteases are associated with the haemophilia metabolic pathway, otherwise known as the coagulation cascade: Thrombin (coagulation factor IIa-Factor IIa), Coagulation factor IXa (Factor IXa), Coagulation factor Xa (Factor Xa), Coagulation factor XIa (Factor XIa), Coagulation factor XIIIa (Factor XIIIa) and Activated protein C (APC).

The Factor Xa protease specifically cleaves after the amino acid sequence P4, P3, P2-Gly, P1-Arg, wherein P4 is Ile, Leu, Pro or Ala and P3 is Glu, Asp, Gln or Asn. Factor Xa preferably cuts after the cleavage sequence Ile, Glu, Gly, Arg. The P1′-P4′ position amino acids may not be determinative for cleavage specificity of Factor Xa.

Thrombin is one of the most extensively studied of all proteases. Its central role in the coagulation cascade as well as several other areas has been thoroughly documented. Thrombin regulates the coagulation process both positively, by cleaving prothrombin, FV and FVIII and negatively, by cleaving protein C. The thrombin cleavage recognition site has been extensively studied (see for example, Gallwitz et al. The Extended Cleavage Specificity of Human Thrombin, (2012) Plos One; 7, e31756). The consensus recognition sequence has been identified as, P4—aliphatic, P3—not negatively charged aa, P2—Pro, P1—Arg, P1′—Ser/Ala/Gly/Thr, P2′—not acidic, P3′—Arg and P4—unspecific aa.

In a preferred embodiment of the invention, the self-regulation element may comprise one of the following thrombin cleavage recognition site sequences: LTPRGVRL (SEQ ID NO: 1), LTPRLVRL (SEQ ID NO: 2), LTPRDVRL (SEQ ID NO: 3), or WYLRSNNG (SEQ ID NO: 4), where cleavage occurs between the two amino acids in bold. In a particularly preferred embodiment the self-regulation element consists of the sequence LTPRGVRL (SEQ ID NO: 1).

By way of further illustration, T cells are involved in the immune system's response to infection, and to the immune system's response to diseases such as cancer, or to conditions such as transplant rejection. In diseases or conditions associated with metabolic pathways where T cells are activated (for example, adoptive immunotherapy), the following proteases can be involved: members of the Granzyme family (Granzymes A, B, K, H, M), Members of the Cathepsin family (including Cathepsin B, L, W), and members of the matrix metalloprotease family (including MMP-2, MMP-9, MMP-28).

Granzymes are granule-stored serine proteases that are implicated in T cell and natural killer cell-mediated cytotoxic defence reactions after target cell recognition. The principal function of granzymes is to induce the death of virus-infected and other potentially harmful cells. Granzyme B is one type of granzyme, and upon target cell contact it is directionally exocytosed and enters target cells assisted by perforin (a cytolytic protein expressed by cytotoxic T cells and natural killer cells). Granzyme B processes and activates various pro-caspases, thereby inducing apoptosis in the target cell. In accordance with the invention, the term “Granzyme B protease” includes enzymes which are or may be classified under the Enzyme Commission number EC 3.4.21.79 in Enzyme nomenclature database, Release 34, February 2004 (‘http://www.expasv.orq/enzvme).

The Granzyme B protease is known to have a preference for cleaving after aspartate residues (D), and Granzyme B is the only mammalian serine protease known to have this P1-proteolytic specificity. Hence, in accordance with the invention it is contemplated that the Granzyme B cleavage site in useful embodiments at least comprises an aspartate residue at the P1 position located N-terminally to the cleavage site. Some of the presently known Granzyme B protease recognition sites are disclosed in Harris et al (1998) Definition and redesign of the extended substrate specificity of granzyme B, J Biol Chem: 273 pp. 27364-73.

Thus, in embodiments of the invention, the Granzyme B cleavage recognition site has an amino acid sequence of the general formula: P4-P3-P2-P1-site of cleavage located N-terminally to the cleavage site, wherein P4 preferably is amino acid I or V, P3 preferably is amino acid E, Q or M, P2 is X, where X denotes any amino acid, P1 preferably is amino acid D.

In a preferred embodiment of the invention, the self-regulation element may comprise one of the following Granzyme B recognition site sequences: IEPDSESQ (SEQ ID NO: 5), IEADSESQ (SEQ ID NO: 6), IEPDSLEE (SEQ ID NO: 7), HSRLGRIEADSESQEDIIRN (SEQ ID NO: 8), VEPDSLEE (SEQ ID NO: 9), LEPDSLEE (SEQ ID NO: 10), IEGDSLEE (SEQ ID NO: 11), IEADSLEE (SEQ ID NO: 12), IEPDALEE (SEQ ID NO: 13), IEPDSEEQ (SEQ ID NO: 14), IEPDSEVE (SEQ ID NO: 15), IEPNSLEE (SEQ ID NO: 16), IEPESLEE (SEQ ID NO: 17), or IEPQSLEE (SEQ ID NO: 18). In a particularly preferred embodiment the self-regulation element consists of the sequence IEPDSLEE (SEQ ID NO: 7).

In a preferred embodiment of the invention, the rate of cleavage of the self-regulation element by the endogenous factor is correlated with the level of activity of the therapeutic agent. The rate of cleavage of the self-regulation element can be calculated according to standard laboratory techniques, such as use of western blotting to visualise protein fragments released over time. The level of activity of the therapeutic agent can be calculated using standard laboratory techniques, such as extent of binding to a target via analysis of Kd. Further examples of suitable techniques are discussed in the Examples of the present application.

In a preferred embodiment of the invention, the rate of cleavage of the self-regulation element by the endogenous factor is correlated with the level of activity of the therapeutic agent, and the amino acid sequence of the self-regulation element. The rate of cleavage of the self-regulation element by the endogenous factor can be tuned by altering the amino acid sequence of the self-regulation element.

### Polypeptide

In a preferred embodiment of the invention, the therapeutic agent comprises a polypeptide, or protein. In an embodiment, the therapeutic agent of the invention consists essentially of a polypeptide.

A polypeptide, or protein, can be defined as any chain of amino acids, regardless of length or post-translational modification (e.g., glycosylation or phosphorylation). In a preferred embodiment of the invention, the polypeptide is an antigen-binding protein. In a preferred embodiment of the invention, the polypeptide is an antibody or a fragment thereof. A “residue” refers to an amino acid or amino acid mimetic incorporated in a polypeptide by an amide bond or amide bond mimetic. A polypeptide has an amino terminal (N-terminal) end and a carboxy terminal end. Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

### Antigen-Binding Protein

An antigen can be defined as a compound, composition, or substance that can stimulate the production of antibodies or a T cell response in an animal, including compositions that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or non-contiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. Antigens can include peptides derived from a pathogen of interest or from a cancerous cell. Exemplary pathogens include bacteria, fungi, viruses and parasites. In some embodiments, an antigen is derived from a cancerous cell such as a haematological cancerous cell (chronic lymphocytic leukaemia—CLL, acute lymphoblastic leukaemia, mantle cell lymphoma) or a solid malignancy (breast, pancreatic, melanoma). In some preferred embodiments, the antigen is a ROR1 polypeptide or antigenic fragment thereof.

A “target epitope” is a specific epitope on an antigen that specifically binds an antibody of interest, such as a monoclonal antibody. In some examples, a target epitope includes the amino acid residues that contact the antibody of interest, such that the target epitope can be selected by the amino acid residues determined to be in contact with the antibody.

In a preferred embodiment of the invention, the therapeutic agent comprises an antigen-binding protein. In a preferred embodiment of the invention, the therapeutic agent is an antigen-binding protein. In a preferred embodiment, the antigen-binding protein of the invention comprises a first antigen binding domain, wherein the first antigen binding domain comprises a heavy chain variable domain comprising a heavy chain complementarity determining region (HCDR)1, an HCDR2 and an HCDR3, and wherein the self-regulation element is positioned C-terminal to the first antigen binding domain. In a preferred embodiment, the first antigen binding domain is a VHH antibody.

In a preferred embodiment of the invention, the antigen binding protein comprises a first antigen binding domain, wherein the first antigen binding domain comprises a light chain variable domain and a heavy chain variable domain, wherein the light chain variable domain comprises a light chain complementarity determining region (LCDR)1, an LCDR2 and an LCDR3, and wherein the heavy chain variable domain comprises a heavy chain complementarity determining region (HCDR)1, an HCDR2 and an HCDR3, and wherein the self-regulation element is positioned C-terminal to the first antigen binding domain.

In an embodiment, the self-regulation element may be adjacent to, and C terminal to, the first antigen-binding domain. The term “adjacent to” implies that the self-regulation element is positioned such that it does not affect the function of the first antigen-binding domain, but it is placed next to the first antigen-binding domain. The self-regulation element may be positioned within a peptide linker sequence. A linker sequence may be at least 1, 2, 3, 4, 5, 6, 7, 8, 9 or 10 amino acids in length. Exemplary linkers include the amino acid sequence GGGGS (SEQ ID NO. 59) and GGGGSGGGGS (SEQ ID NO. 60).

In a preferred embodiment, the antigen binding protein forms part of a Chimeric antigen receptor (CAR) or T cell receptor (TCR). In a preferred embodiment, the antigen binding protein is exogenously expressed in a CAR-T cell, a CAR-M cell or a modified NK cell.

The antigen binding proteins of the invention include antibodies. The antigen binding proteins of the invention include single chain antibodies (i.e. a full-length heavy chain and light chain); Fab, modified Fab, Fab′, modified Fab′, F(ab′)2, Fv, Fab-Fv, Fab-dsFv, single domain antibodies (e.g. VH or VL or VHH), scFv, mono-, bi-, tri- or tetra-valent antibodies, scFv, Bis-scFv, single-domain antibodies (sdAbs), also known as VHH antibodies, nanobodies (Camelid-derived single-domain antibodies), shark IgNAR-derived single-domain antibody, diabodies, tribodies, triabodies, tetrabodies and epitope-binding fragments of any of the above (see for example Holliger P and Hudson P J, 2005, Nat. Biotechnol., 23: 1126-1136; Adair J R and Lawson A D G, 2005, Drug Design Reviews—Online, 2, 209-217). The methods for creating and manufacturing these antigen-binding proteins are well known in the art (see for example Verma R et al., 1998, J. Immunol. Methods, 216, 165-181). The Fab-Fv format was first disclosed in WO2009/040562 and the disulphide-stabilised versions thereof, the Fab-dsFv was first disclosed in WO2010/035012. Multi-valent antigen-binding proteins of the invention may comprise multiple specificities e.g. bispecific, or may be monospecific. The antigen binding proteins of the invention may be biparatopic.

A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies and heteroconjugate antibodies such as bispecific antibodies. See also, Pierce Catalog and Handbook, 1994-1995 (Pierce Chemical Co., Rockford, IL); Kuby, Immunology, 3^{rd} Ed., W.H. Freeman & Co., New York, 1997.

Antigen binding proteins of the invention include, but are not limited to, the following: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)₂, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule.

In an embodiment of the invention, the antigen binding protein may comprise heavy and light chain each containing a constant region and a variable region, (the regions are also known as “domains”). In several embodiments of the invention, the heavy and the light chain variable domains combine to specifically bind the antigen. In additional embodiments of the invention, only the heavy chain variable domain is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). Light and heavy chain variable domains contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for antigen binding. The sequences of the framework regions of different light or heavy chains are relatively conserved within a species. The framework region of an antibody, that is the combined framework regions of the constituent light and heavy chains, serves to position and align the CDRs in three-dimensional space.

The CDRs of each chain are typically referred to as CDR1, CDR2, and CDR3 (from the N-terminus to C-terminus), and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Light chain CDRs can also be referred to as CDR L1, CDR L2 and CDR L3, or LCDR1, LCDR2 and LCDR3. Heavy chain CDRs can be referred to as CDR H1, CDR H2 and CDR H3, or HCDR1, HCDR2 and HCDR3.

The residues in antibody variable domains are conventionally numbered according to IMGT (http://www.imgt.org). This system is set forth in Lefranc M P (1997, J, Immunol. Today, 18, 509). This numbering system is used in the present specification except where otherwise indicated.

The IMGT residue designations do not always correspond directly with the linear numbering of the amino acid residues. The actual linear amino acid sequence may contain fewer or additional amino acids than in the strict IMGT numbering corresponding to a shortening of, or insertion into, a structural component, whether framework or CDR, of the basic variable domain structure. The correct IMGT numbering of residues may be determined for a given antibody by alignment of residues of homology in the sequence of the antibody with a “standard” IMGT numbered sequence.

Suitable antigen binding proteins, antibodies or binding fragments thereof may be disclosed herein by the primary amino acid sequences of their heavy and light chain CDRs, their heavy and light chain variable regions, and/or their full length heavy and light chains.

An antigen binding protein, antibody or binding fragment thereof may comprise one or more VH CDR sequences and alternatively or additionally one or more VL CDR sequences of said specific antigen binding protein or antibody, in addition to VL CDR1. An antigen binding protein, antibody or binding fragment thereof may comprise one, two or all three of the VH CDR sequences of a specific antigen binding protein, antibody or binding fragment thereof as described above and alternatively or additionally one, two or all three of the VL chain CDR sequences of said specific antigen binding protein, antibody or binding fragment thereof, including VL CDR1. An antigen binding protein, antibody or binding fragment thereof may comprise all six CDR sequences of a specific antigen binding protein, antibody or binding fragment as described above.

A variant antigen binding protein or antibody may comprise 1, 2, 3, 4, 5, up to 10, up to 20, up to 30 or more amino acid substitutions and/or deletions from the specific sequences and fragments discussed above, whilst maintaining the activity of the antigen binding proteins or antibodies described herein. “Deletion” variants may comprise the deletion of, for example, 1, 2, 3, 4 or 5 individual amino acids or of one or more small groups of amino acids such as 2, 3, 4 or 5 amino acids. “Small groups of amino acids” can be defined as being sequential, or in close proximity but not sequential, to each other. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid, another aliphatic amino acid, another tiny amino acid, another small amino acid or another large amino acid. Some properties of the 20 main amino acids, which can be used to select suitable substituents, are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged (+) | Trp | aromatic, hydrophobic, neutral |
| Leu | aliphatic, hydrophobic, neutral | Tyr | aromatic, polar, hydrophobic |

Preferred “derivatives” or “variants” include those in which instead of the naturally occurring amino acid the amino acid, which appears in the sequence, is a structural analog thereof. Amino acids used in the sequences may also be derivatized or modified, e.g. labelled, providing the function of the antibody is not significantly adversely affected.

Derivatives and variants as described above may be prepared during synthesis of the antigen binding protein or antibody or by post-production modification, or when the antigen binding protein or antibody is in recombinant form using the known techniques of site-directed mutagenesis, random mutagenesis, or enzymatic cleavage and/or ligation of nucleic acids.

Preferably variant antigen bonding proteins or antibodies have an amino acid sequence which has more than 60%, or more than 70%, e.g. 75 or 80%, preferably more than 85%, e.g. more than 90%, 95%, 96%, 97%, 98% or 99% amino acid identity to the VL and/or VH, or a fragment thereof, of an antigen binding protein or antibody disclosed herein. This level of amino acid identity may be seen across the full-length of the relevant SEQ ID NO sequence or over a part of the sequence, such as across 20, 30, 50, 75, 100, 150, 200 or more amino acids, depending on the size of the full-length polypeptide.

Preferably the variant antigen binding proteins or antibodies comprise one or more of the CDR sequences as described herein.

In connection with amino acid sequences, “sequence identity” refers to sequences, which have the stated value when assessed using ClustalW (Thompson J D et al., 1994, Nucleic Acid Res., 22, 4673-4680) with the following parameters:
Pairwise alignment parameters—Method: slow/accurate, Matrix: PAM, Gap open penalty: 10.00, Gap extension penalty: 0.10;
Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: G, P, S, N, D, Q, E, K, R. Sequence identity at a particular residue is intended to include identical residues, which have simply been derivatized.

The methods of the present invention may use antibodies having specific VH and VL amino acid sequences and variants and fragments thereof, which maintain the function or activity of these VHs and VLs.

References to “V_{H}” or “VH” refer to the variable region of an immunoglobulin heavy chain, including that of an antibody fragment, such as Fv, scFv, dsFv or Fab. References to “V_{L}” or “VL” refer to the variable region of an immunoglobulin light chain, including that of an Fv, scFv, dsFv or Fab.

In some embodiments of the invention, the antigen binding protein may be an antibody comprising heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

IgG1 (e.g. IgG1/kappa) antibodies having an IgG1 heavy chain and a light chain may advantageously be used in the invention. However, other human antibody isotypes are also encompassed by the invention, including IgG2, IgG3, IgG4, IgM, IgA1, IgA2, IgAsec, IgD and IgE in combination with a kappa or lambda light chain. Also, all animal-derived antibodies of various isotypes can be used in the invention. The antibodies can be full-size antibodies or antigen-binding fragments of antibodies, including Fab, F(ab′)2, single-chain Fv fragments, or single-domain VHH, VH or VL single domains.

The Fc region generally refers to the last two constant region immunoglobulin domains of IgA, IgD, and IgG, and the last three constant region immunoglobulin domains of IgE and IgM. An Fc region may also include part or all of the flexible hinge N-terminal to these domains. For IgA and IgM, an Fc region may or may not comprise the tailpiece, and may or may not be bound by the J chain. For IgG, the Fc region comprises immunoglobulin domains Cgamma2 and Cgamma3 (Cγ2 and Cγ3) and the lower part of the hinge between Cgamma1 (Cγ1) and Cγ2. Although the boundaries of the Fc region may vary, the human IgG heavy chain Fc region is usually defined to comprise residues C226 or P230 to its carboxyl-terminus, wherein the numbering is according to the EU index. For IgA, the Fc region comprises immunoglobulin domains Calpha2 and Calpha3 (Cα2 and Cα3) and the lower part of the hinge between Calpha1 (Cα1) and Cα2. Encompassed within the definition of the Fc region are functionally equivalent analogs and variants of the Fc region. A functionally equivalent analog of the Fc region may be a variant Fc region, comprising one or more amino acid modifications relative to the wild-type or naturally existing Fc region. Variant Fc regions will possess at least 50% homology with a naturally existing Fc region, such as about 80%, and about 90%, or at least about 95% homology. Functionally equivalent analogs of the Fc region may comprise one or more amino acid residues added to or deleted from the N- or C-termini of the protein, such as no more than 30 or no more than 10 additions and/or deletions. Functionally equivalent analogs of the Fc region include Fc regions operably linked to a fusion partner. Functionally equivalent analogs of the Fc region must comprise the majority of all of the Ig domains that compose Fc region as defined above; for example, IgG and IgA Fc regions as defined herein must comprise the majority of the sequence encoding CH₂ and the majority of the sequence encoding CH₃. Thus, the CH₂ domain on its own, or the CH₃ domain on its own, are not considered Fc region. The Fc region may refer to this region in isolation, or this region in the context of an Fc fusion polypeptide.

When referring to an antigen binding protein of the invention, the binding to said protein to an antigen refers to a binding reaction which determines the presence of a target protein, peptide, or polysaccharide in the presence of a heterogeneous population of proteins and other biologics. Thus, under designated conditions, an antigen binding protein binds preferentially to a particular target protein, peptide or polysaccharide (such as an antigen present on the surface of a tumour, for example ROR1) and does not bind in a significant amount to other proteins or polysaccharides present in the sample or subject. Specific binding can be determined by methods known in the art. With reference to an antibody antigen complex, specific binding of the antigen and antibody has a K_{d} of less than about 10⁻⁵ Molar, 10⁻⁶ Molar, 10⁻⁷ Molar, such as less than about 10⁻⁷ Molar, 10⁻⁸ Molar, 10⁻⁹, or even less than about 10⁻¹⁰ Molar.

The terms “binding activity” and “binding affinity” are intended to refer to the tendency of an antigen-binding protein to bind or not to bind to a target. Binding affinity may be quantified by determining the dissociation constant (Kd) for an antigen-binding protein and its target. Similarly, the specificity of binding of an antigen binding protein to its target may be defined in terms of the comparative dissociation constants (Kd) of the antibody for its target as compared to the dissociation constant with respect to the antigen binding protein and another, non-target molecule.

Typically, the Kd for the antibody with respect to the target will be 2-fold, preferably 5-fold, more preferably 10-fold less than the Kd with respect to the other, non-target molecule such as unrelated material or accompanying material in the environment. More preferably, the Kd will be 50-fold less, even more preferably 100-fold less, and yet more preferably 200-fold less.

The value of this dissociation constant can be determined directly by well-known methods, and can be computed even for complex mixtures by methods such as those, for example, set forth in Caceci M S and Cacheris W P (1984, Byte, 9, 340-362). For example, the Kd may be established using a double-filter nitrocellulose filter binding assay such as that disclosed by Wong I and Lohman T M (1993, Proc. Natl. Acad. Sci. USA, 90, 5428-5432) or for example, by using Octet surface plasmon resonance.

One method for the evaluation of binding affinity is by ELISA. Other standard assays to evaluate the binding ability of ligands such as antibodies towards targets are known in the art, including for example, Western blots, RIAs, and flow cytometry analysis. The binding kinetics (e.g. binding affinity) of the antibody also can be assessed by standard assays known in the art, such as surface plasmon resonance, for example by Biacore™ system analysis.

In one embodiment the antigen binding protein is a monoclonal antibody. Monoclonal antibodies are immunoglobulin molecules that are identical to each other and have a single binding specificity and affinity for a particular epitope. Monoclonal antibodies (mAbs) of the present invention can be produced by a variety of techniques, including conventional monoclonal antibody methodology, for example those disclosed in “Monoclonal Antibodies: a manual of techniques” (Zola H, 1987, CRC Press) and in “Monoclonal Hybridoma Antibodies: techniques and applications” (Hurrell J G R, 1982 CRC Press).

In a preferred embodiment of the invention, the therapeutic agent is an antigen binding protein comprising a first antigen binding domain and a second binding domain, wherein the self-regulation element is positioned between the first and second antigen binding domains. The term “between”, can be interpreted to mean that the self-regulation element is positioned C-terminal to the first antigen binding domain and N-terminal to the second antigen binding domain. In an embodiment of the invention, the antigen binding protein contains more than one self-regulation element. In a preferred embodiment of the invention, the antigen binding protein is a bi-specific antibody comprising a first self-regulation element C terminal to the first antigen binding domain, and a second self-regulation element N terminal to the second antigen binding domain.

In an embodiment of the invention where the antigen binding protein comprises an scFv, the self-regulation element may be positioned within the scFv in a peptide linker sequence between the heavy and light chain variable regions, so that the scFv becomes non-functional when the self-regulation element is cleaved.

In a preferred embodiment of the invention, the first and second antigen binding domains of the antigen binding protein are scFv proteins covalently linked by a peptide linker. In preferred embodiment, the self-regulation element is present within the peptide linker.

As indicated below, the sequence of each light chain variable domain and heavy chain variable domain referred to above may differ from the given sequence. For example, the light/heavy chain variable domain may comprise a sequence which is at least 90%, 91%, 92%, 93%, 94%, 95%, 96%, 97%, 98% or 99% identical to the amino acid sequences as set forth in the sequence listing. Alternatively, the light/heavy chain variable domain sequence may differ at up to 10 amino acid positions, although it is preferred that fewer than 10 amino acid substitutions are present so that there may be up to 9, 8, 7, 6, 5, 4, 3, 2 or 1 amino acid substitutions.

As referred to above, in some embodiments, the Light Chain Variable Domains and the Heavy Chain Variable Domains comprise an amino acid sequence at least 90%, 91%, 92%, 93%, 94%, 95%, 96%, 97%, 98% or 99% identical to the amino acid sequences as set forth above. For example, the Light Chain Framework Regions, the Heavy Chain Framework Regions, the Light Chain Variable Domains and the Heavy Chain Variable Domains may include at most 10, at most 9, at most 8, at most 7, at most 6, at most 5, at most 4, at most 3, at most two or at most one amino acid substitutions in the amino acid sequences as set forth above. Where there is variation in the sequences of the Light Chain Variable Domain and the Heavy Chain Variable Domain, any amino acid substitutions are preferably not in the CDRs. In particular, the Light Chain Framework Regions and/or the Heavy Chain Framework Regions of the antibodies described above may comprise an amino acid sequence at least 90%, 91%, 92%, 93%, 94%, 95%, 96%, 97%, 98% or 99% identical to the amino acid sequences as set forth above. Further, the Light Chain Framework Regions and/or the Heavy Chain Framework Regions may include at most 10, at most 9, at most 8, at most 7, at most 6, at most 5, at most 4, at most 3, at most two or at most one amino acid substitutions in the amino acid sequences as set forth above. Preferably the amino acid substitutions are conservative substitutions as described above. For example, the framework regions may comprise such substitutions in order to humanise the sequence. Preferably, the framework regions are humanised.

The sequence of each light chain variable domain and heavy chain variable domain of the second antigen binding domain referred to above may differ from the given sequence. For example, the light/heavy chain variable domain may comprise a sequence which is at least 90%, 91%, 92%, 93%, 94%, 95%, 96%, 97%, 98% or 99% identical to the amino acid sequences as set forth in the sequence listing. Alternatively, the light/heavy chain variable domain sequence may differ at up to 10 amino acid positions, although it is preferred that fewer than 10 amino acid substitutions are present so that there may be up to 9, 8, 7, 6, 5, 4, 3, 2 or 1 amino acid substitutions. Preferably, there are no substitutions present in the CDRs of the heavy/light chain.

Antigen binding proteins comprising the antigen binding domains can be prepared by proteolytic hydrolysis of the antibody or by expression in mammalian cells of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. Nos. 4,036,945 and 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antigen binding proteins and antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody, or antigen binding protein.

In an embodiment of the invention, the antigen binding protein is exogenously expressed in a CAR-T cell, a CAR-M cell or a modified NK cell.

Position of Cleavage Site within Antigen Binding Protein

In an embodiment of the invention, the self-regulation element is positioned in an exposed position within the antigen-binding protein, so that the self-regulation element is accessible to the endogenous factor.

In an embodiment of the invention, the antigen binding protein comprises a self-regulation element, wherein the self-regulation element is positioned within a peptide linker sequence.

In an embodiment of the invention, the antigen binding protein comprises a self-regulation element, wherein the self-regulation element is positioned within a peptide linker sequence within the first and/or the second antigen binding domains.

In an embodiment, the self-regulation element may be adjacent to, and C terminal to, the first antigen-binding domain. The term “adjacent to” implies that the self-regulation element is positioned such that it does not affect the function of the first antigen-binding domain, but it is placed within about 2, 3, 4, 5, 6, 7, 8, 9, or 10 amino acids of the first antigen-binding domain. The self-regulation element may be positioned within a peptide linker sequence. A linker sequence may be at least 1, 2, 3, 4, 5, 6, 7, 8, 9 or 10 amino acids in length. Exemplary linkers include the amino acid sequence GGGGS (SEQ ID NO. 59) and GGGGSGGGGS (SEQ ID NO. 60). In an embodiment of the invention, the self-regulation element is positioned directly C-terminal to a peptide linker with sequence GGGGS (SEQ ID NO. 59).

In an embodiment of the invention, the self-regulation element may be positioned between the first and the second antigen binding domains of the antigen binding protein. In an embodiment, the self-regulation element may be positioned in a hinge region present between the first and the second antigen binding domains.

In an embodiment of the invention, the antigen binding protein comprises a constant region, and the self-regulation element is located within the hinge region of the constant region, between CH1 and CH2. In a preferred embodiment, there are two self-regulation elements, each one located within the hinge region of the constant region, between CH1 and CH2.

### ROR1-Binding Antigen Binding Proteins

In a preferred embodiment, the antigen binding protein of the invention comprises a self-regulation element comprising a Granzyme B cleavage site. In a preferred embodiment of the invention the antigen binding protein comprises a first antigen binding domain which selectively binds to Receptor Tyrosine Kinase Like Orphan Receptor 1 (ROR1) (also known as Neurotrophic Tyrosine Kinase, Receptor-Related 1, NTRKR1), wherein the first antigen binding domain binds to an epitope of ROR1 comprising amino acid Gln-261, and a self-regulation element comprising a Granzyme B cleavage site.

In a preferred embodiment of the invention the antigen binding protein comprises a first antigen binding domain which selectively binds to Receptor Tyrosine Kinase Like Orphan Receptor 1 (ROR1) (also known as Neurotrophic Tyrosine Kinase, Receptor-Related 1, NTRKR1), wherein the first antigen binding domain binds to an epitope of ROR1 comprising amino acid Gln-261; and
a second antigen binding domain which selectively binds to the CD3 subunit of the T-Cell Receptor (TCR), and a self-regulation element comprising a Granzyme B cleavage site. In a more preferred embodiment, the self-regulation element is set out in SEQ ID NO 7.

Antigen binding proteins disclosed herein that specifically bind to ROR1 and CD3 are disclosed in WO2019/008379, which is incorporated herein by reference.

In a first embodiment, the first antigen binding domain comprises a light chain variable domain and a heavy chain variable domain, wherein the light chain variable domain comprises a light chain complementarity determining region (LCDR)1, an LCDR2 and an LCDR3, wherein LCDR1 comprises the amino acid sequence set forth in SEQ ID NO: 19; LCDR2 comprises the amino acid sequence set forth in SEQ ID NO: 20; and LCDR3 comprises the amino acid sequence set forth in SEQ ID NO: 21; and wherein the heavy chain variable domain comprises a heavy chain complementarity determining region (HCDR)1, an HCDR2 and an HCDR3, wherein HCDR1 comprises the amino acid sequence set forth in SEQ ID NO: 22; HCDR2 comprises the amino acid sequence set forth in SEQ ID NO: 23; and HCDR3 comprises the amino acid sequence set forth in SEQ ID NO: 24; wherein the sequence of each complementarity determining region may differ from the given sequence at up to two amino acid positions.

The antigen binding protein specifically binds to a ROR1 polypeptide and specifically binds to CD3. The first and/or second antigen binding domain may be a monoclonal antibody or an antigen binding fragment thereof. In particular embodiments both the first and second antigen binding domains are a monoclonal antibody or an antigen binding fragment thereof.

As indicated above, the sequence of each CDR may differ from the given sequence at up to two amino acid positions. This means that the CDR may contain one or two amino acid substitutions compared to the given sequence. However, if one or more of the CDRs does contain amino acid substitutions, the antibody can still selectively bind to ROR1. Preferably, the amino acid substitutions are conservative substitutions.

Preferably, the sequence of each CDR may differ from the given sequence at one amino acid position. This means that the CDR may contain one amino acid substitution compared to the given sequence. Preferably, the amino acid substitution is a conservative substitution.

In some embodiments, heavy chain complementarity determining region 3 (HCDR3) comprises an amino acid sequence selected from any of the sequences set forth in SEQ ID NOs: 25, 26, 27 and 28. Preferably, HCDR3 comprises an amino acid sequence selected from any of the sequences set forth in SEQ ID NOs: 26, 27 and 28.

Preferably, the light chain variable domain comprises the amino acid sequence as set forth as one of SEQ ID NOs: 29, 30, 31, 32, 33 and 34. More preferably, the light chain variable domain comprises the amino acid sequence as set forth as one of SEQ ID NOs: 30, 31, 32, 33 and 34.

Preferably, the heavy chain variable domain comprises the amino acid sequence as set forth as one of SEQ ID NOs: 35, 36, 37, 38, 39 and 40. More preferably, the heavy chain variable domain comprises the amino acid sequence as set forth as one of SEQ ID NOs: 36, 37, 38, 39 and 40.

SEQ ID NOs: 30, 31, 32, 33 and 34 are humanised light chain variable regions. SEQ ID NOs: 36, 37, 38, 39 and 40 are humanised light chain variable regions. The inventors of WO2019/008379 tried all combinations of these light and heavy chain regions resulting in 25 different constructs.

Therefore, in some embodiments, the light chain variable domain comprises the amino acid sequence as set forth as SEQ ID NO: 30 and the heavy chain variable domain comprises the amino acid sequence as set forth as one of SEQ ID NOs: 36, 37, 38, 39 and 40. In a particular embodiment, the light chain variable domain comprises the amino acid sequence as set forth as SEQ ID NO: 30 and the heavy chain variable domain comprises the amino acid sequence as set forth as one of SEQ ID NOs: 36, 38 and 39.

In other embodiments, the light chain variable domain comprises the amino acid sequence as set forth as SEQ ID NO: 31 and the heavy chain variable domain comprises the amino acid sequence as set forth as one of SEQ ID NOs: 36, 37, 38, 39 and 40.

In further embodiments, the light chain variable domain comprises the amino acid sequence as set forth as SEQ ID NO: 32 and the heavy chain variable domain comprises the amino acid sequence as set forth as one of SEQ ID NOs: 36, 37, 38, 39 and 40.

In alternative embodiments, the light chain variable domain comprises the amino acid sequence as set forth as SEQ ID NO: 33 and the heavy chain variable domain comprises the amino acid sequence as set forth as one of SEQ ID NOs: 36, 37, 38, 39 and 40.

In various embodiments, the light chain variable domain comprises the amino acid sequence as set forth as SEQ ID NO: 34 and the heavy chain variable domain comprises the amino acid sequence as set forth as one of SEQ ID NOs: 36, 37, 38, 39 and 40.

Similarly, in some embodiments, the heavy chain variable domain comprises the amino acid sequence as set forth as SEQ ID NO: 36 and the light chain variable domain comprises the amino acid sequence as set forth as one of SEQ ID NOs: 30, 31, 32, 33 and 34.

In other embodiments, the heavy chain variable domain comprises the amino acid sequence as set forth as SEQ ID NO: 37 and the light chain variable domain comprises the amino acid sequence as set forth as one of SEQ ID NOs: 30, 31, 32, 33 and 34.

In further embodiments, the heavy chain variable domain comprises the amino acid sequence as set forth as SEQ ID NO: 38 and the light chain variable domain comprises the amino acid sequence as set forth as one of SEQ ID NOs: 30, 31, 32, 33 and 34.

In alternative embodiments, the heavy chain variable domain comprises the amino acid sequence as set forth as SEQ ID NO: 39 and the light chain variable domain comprises the amino acid sequence as set forth as one of SEQ ID NOs: 30, 31, 32, 33 and 34.

In various embodiments, the heavy chain variable domain comprises the amino acid sequence as set forth as SEQ ID NO: 40 and the light chain variable domain comprises the amino acid sequence as set forth as one of SEQ ID NOs: 30, 31, 32, 33 and 34.

In particular embodiments,
(a) the light chain variable domain comprises the amino acid sequence as set forth as SEQ ID NO: 29 and the heavy chain variable domain comprises the amino acid sequence as set forth as SEQ ID NO: 35;
(b) the light chain variable domain comprises the amino acid sequence as set forth as SEQ ID NO: 30 and the heavy chain variable domain comprises the amino acid sequence as set forth as SEQ ID NO: 36;
(c) the light chain variable domain comprises the amino acid sequence as set forth as SEQ ID NO: 31 and the heavy chain variable domain comprises the amino acid sequence as set forth as SEQ ID NO: 37;
(d) the light chain variable domain comprises the amino acid sequence as set forth as SEQ ID NO: 32 and the heavy chain variable domain comprises the amino acid sequence as set forth as SEQ ID NO: 38;
(e) the light chain variable domain comprises the amino acid sequence as set forth as SEQ ID NO: 33 and the heavy chain variable domain comprises the amino acid sequence as set forth as SEQ ID NO: 39; or
(f) the light chain variable domain comprises the amino acid sequence as set forth as SEQ ID NO: 34 and the heavy chain variable domain comprises the amino acid sequence as set forth as SEQ ID NO: 40.

The second antigen binding domain which selectively binds to the CD3 subunit of the T-Cell Receptor (TCR) can be any suitable antigen binding domain and such binding domains are well known to those skilled in the art. For example, CD3 monoclonal antibodies can be obtained from ThermoFisher Scientific. Further, bispecific antibodies which bind to a tumour antigen and CD3 are also known in the art, for example, as described in Baeuerle and Reinhardt (Cancer Res (2009); 69(12): 4941-4944), Chames and Baty (MAbs. (2009); 1(6): 539-547) and Hoffman et al. (Int. J. Cancer (2005) 115, 98-104).

The second antigen binding domain which selectively binds to the CD3 subunit of the T-Cell Receptor (TCR) may comprise a light chain variable domain and a heavy chain variable domain, wherein the light chain variable domain comprises the amino acid sequence as set forth as one of SEQ ID NOs: 41, 42, 43, 44, 45 and 46, and wherein the heavy chain variable domain comprises the amino acid sequence as set forth as one of SEQ ID NOs: 47, 48, 49, 50, 51 and 52. Preferably, the light chain variable domain comprises the amino acid sequence as set forth as one of SEQ ID NOs: 42, 43, 44, 45 and 46, and the heavy chain variable domain comprises the amino acid sequence as set forth as one of SEQ ID NOs: 48, 49, 50, 51 and 52.

In particular embodiments of the second antigen binding domain,
(a) the light chain variable domain comprises the amino acid sequence as set forth as SEQ ID NO: 41 and the heavy chain variable domain comprises the amino acid sequence as set forth as SEQ ID NO: 47;
(b) the light chain variable domain comprises the amino acid sequence as set forth as SEQ ID NO: 42 and the heavy chain variable domain comprises the amino acid sequence as set forth as SEQ ID NO: 48;
(c) the light chain variable domain comprises the amino acid sequence as set forth as SEQ ID NO: 43 and the heavy chain variable domain comprises the amino acid sequence as set forth as SEQ ID NO: 49;
(d) the light chain variable domain comprises the amino acid sequence as set forth as SEQ ID NO: 44 and the heavy chain variable domain comprises the amino acid sequence as set forth as SEQ ID NO: 50;
(e) the light chain variable domain comprises the amino acid sequence as set forth as SEQ ID NO: 45 and the heavy chain variable domain comprises the amino acid sequence as set forth as SEQ ID NO: 51; or
(f) the light chain variable domain comprises the amino acid sequence as set forth as SEQ ID NO: 46 and the heavy chain variable domain comprises the amino acid sequence as set forth as SEQ ID NO: 52.

In a first embodiment, the second antigen binding domain comprises a light chain variable domain and a heavy chain variable domain, wherein the light chain variable domain comprises a light chain complementarity determining region (LCDR)1, an LCDR2 and an LCDR3, wherein LCDR1 comprises the amino acid sequence set forth in SEQ ID NO: 53; LCDR2 comprises the amino acid sequence set forth in SEQ ID NO: 54; and LCDR3 comprises the amino acid sequence set forth in SEQ ID NO: 55; and wherein the heavy chain variable domain comprises a heavy chain complementarity determining region (HCDR)1, an HCDR2 and an HCDR3, wherein HCDR1 comprises the amino acid sequence set forth in SEQ ID NO: 56; HCDR2 comprises the amino acid sequence set forth in SEQ ID NO: 57; and HCDR3 comprises the amino acid sequence set forth in SEQ ID NO: 58; wherein the sequence of each complementarity determining region may differ from the given sequence at up to two amino acid positions.

The first antigen binding domain may have the structure of an antibody fragment such as Fab, F(ab′)₂, and Fv which include a heavy chain and light chain variable region and are capable of binding the epitopic determinant on ROR1. Similarly, the second antigen binding domain may have the structure of an antibody fragment such as Fab, F(ab′)₂, and Fv which include a heavy chain and light chain variable region and are capable of binding the epitopic determinant on CD3. These antibody fragments retain the ability to selectively bind with the antigen and are described above. Methods of making these fragments are known in the art (see for example, Harlow and Lane, Antibodies: A Laboratory Manual, Cold Spring Harbor Laboratory, New York, 1988).

In a further group of embodiments, the antigen binding domains may have the structure of an Fv antibody, which are typically about 25 kDa and contain a complete antigen-binding site with three CDRs per each heavy chain and each light chain. To produce these antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as mammalian cells or E. coli. The recombinant host cells synthesize a single polypeptide chain with a linker peptide bridging the two V domains. Exemplary linkers include the amino acid sequence GGGGS (SEQ ID NO. 59) and GGGGSGGGGS (SEQ ID NO. 60). Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio Technology 11:1271, 1993; and Sandhu, supra). Dimers of a single chain antibody (scFV₂), are also contemplated.

The antigen binding proteins disclosed herein can be derivatized or linked to another molecule (such as another peptide or protein). In general, the antigen binding protein is derivatized such that the binding to the ROR1 polypeptide and CD3 subunit are not affected adversely by the derivatization or labelling. For example, the antigen binding protein can be functionally linked, for example, by chemical coupling, genetic fusion, noncovalent association or otherwise to one or more other molecular entities, such as another antibody, a detection agent, a pharmaceutical agent, and/or a protein or peptide that can mediate association of the antibody with another molecule (such as a streptavidin core region or a polyhistidine tag).

A bispecific antibody can be produced by cross-linking two or more antibodies (of the same type or of different types. Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are available, for example, from Pierce Chemical Company (Rockford, IL).

In particular embodiments, the ROR1 antigen binding domain may be a scFv antibody. In some embodiments, the CD3 antigen binding domain is a scFv antibody. In various embodiments, both the ROR1 antigen binding domain and the CD3 antigen binding domain are scFv antibodies. These two scFv antibodies may be covalently linked using a short peptide linker of between 5 and 20 amino acids.

In a preferred embodiment the self-regulation element is located within this short peptide linker.

In some embodiments, the antigen binding protein comprises the sequence of SEQ ID NO. 61 or 62, or a sequence having at least 90% sequence identity thereto. The sequence may have at least 91%, 92%, 93%, 94%, 95%, 96%, 97%, 98% or 99% sequence identity thereto. In some embodiments, the bispecific antibody comprises the sequence of SEQ ID NO. 61 or 62.

The antibody can be labelled with a detectable moiety or marker as described above. The antibody can also be labelled with a radiolabeled amino acid. Examples of radiolabels include, but are not limited to, the following radioisotopes or radionucleotides: ³H, ¹⁴C, ¹⁵N, ³⁵, ⁹⁰Y, ⁹⁹Tc, ¹¹¹In, ¹²⁵ I, ¹³¹I. The radiolabel may be used for both diagnostic and therapeutic purposes.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

In particular embodiments, the ROR1 antigen binding domain may be part of a chimeric antigen receptor (CAR). The CAR may comprise a spacer sequence or hinge region to connect the ROR1-binding domain with the transmembrane domain and spatially separate the ROR1-binding domain from the endodomain of the CAR. A flexible spacer/hinge region allows the ROR1-binding domain to orient in different directions to enable ROR1 binding. Herein “flexible spacer”, “spacer sequence” and “hinge region” are used interchangeably as equivalent terms. A skilled person will appreciate that any suitable spacer sequence can be used. In a preferred embodiment the self-regulation element is located within this flexible spacer/hinge region. In a preferred embodiment the hinge region is set forth as SEQ ID NO: 82. In a preferred embodiment, the self-regulation element is positioned directly N-terminal to the hinge region. In a preferred embodiment the self-regulation element replaces the first 1, 2, 3, 4, or 5 amino acids in the hinge region. In a preferred embodiment, the self-regulation element is a Granzyme B cleavage site. In a preferred embodiment the combined self-regulation element and hinge region are set forth as SEQ ID NO: 83 or 84.

### Factor IXa and X Antigen-Binding Protein

In a preferred embodiment, the antigen binding protein of the invention comprises a self-regulation element comprising a Thrombin cleavage site. In a preferred embodiment, the antigen binding protein comprises a first antigen binding domain that selectively binds to FIX/FIXa and a second antigen binding domain that selectively binds to FX/FXa, and a self-regulation element comprising a Thrombin cleavage site. In a preferred embodiment, the antigen binding protein comprises a first antigen binding domain that selectively binds to FIXa and a second antigen binding domain that selectively binds to FX, and an self-regulation element comprising a Thrombin cleavage site. In a preferred embodiment, the self-regulation element comprises SEQ ID NO: 1, 2, 3 or 4. In a preferred embodiment, the self-regulation element comprises SEQ ID NO: 1.

In a preferred embodiment, the first antigen binding domain and second binding domain each comprise a light chain variable domain and a heavy chain variable domain, wherein the light chain variable domain comprises a light chain complementarity determining region (LCDR)1, an LCDR2 and an LCDR3, and wherein the heavy chain variable domain comprises a heavy chain complementarity determining region (HCDR)1, an HCDR2 and an HCDR3, wherein for the first antigen binding domain LCDR1 comprises the amino acid sequence set forth in SEQ ID NO: 63; LCDR2 comprises the amino acid sequence set forth in SEQ ID NO: 64; and LCDR3 comprises the amino acid sequence set forth in SEQ ID NO: 65; and wherein the heavy chain variable domain comprises a heavy chain complementarity determining region (HCDR)1, an HCDR2 and an HCDR3, wherein HCDR1 comprises the amino acid sequence set forth in SEQ ID NO: 66; HCDR2 comprises the amino acid sequence set forth in SEQ ID NO: 67; and HCDR3 comprises the amino acid sequence set forth in SEQ ID NO: 68; and wherein for the second antigen binding domain LCDR1 comprises the amino acid sequence set forth in SEQ ID NO: 63; LCDR2 comprises the amino acid sequence set forth in SEQ ID NO: 64; and LCDR3 comprises the amino acid sequence set forth in SEQ ID NO: 65; and wherein the heavy chain variable domain comprises a heavy chain complementarity determining region (HCDR)1, an HCDR2 and an HCDR3, wherein HCDR1 comprises the amino acid sequence set forth in SEQ ID NO: 69; HCDR2 comprises the amino acid sequence set forth in SEQ ID NO: 70; and HCDR3 comprises the amino acid sequence set forth in SEQ ID NO: 71;
wherein the sequence of each complementarity determining region may differ from the given sequence at up to two amino acid positions.

In particular embodiments, for the first antigen binding domain the light chain variable domain comprises the amino acid sequence as set forth as SEQ ID NO: 72 and the heavy chain variable domain comprises the amino acid sequence as set forth as SEQ ID NO: 73, and for the second antigen binding domain the light chain variable domain comprises the amino acid sequence as set forth as SEQ ID NO: 72 and the heavy chain variable domain comprises the amino acid sequence as set forth as SEQ ID NO: 74.

In a preferred embodiment, the antigen binding protein comprises a constant region. In a preferred embodiment, the self-regulation element is located within the constant region. In a preferred embodiment, the self-regulation element is located within the hinge region of the constant region. In a preferred embodiment, the self-regulation element is located within the hinge region of the constant region, between CH1 and CH2. In a preferred embodiment, the self-regulation element and hinge region comprise SEQ ID NO: 76. In a preferred embodiment, there are two self-regulation elements, each one located within the hinge region of the constant region, between CH1 and CH2. In a preferred embodiment, the antigen binding protein is Emicizumab, wherein the antigen binding protein also comprises an self-regulation element comprising a Thrombin cleavage site.

The antigen binding protein can be produced by cross-linking two or more antibodies (of the same type or of different types. Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are available, for example, from Pierce Chemical Company (Rockford, TL).

### Polynucleotides, Vectors and Host Cells

The present invention also encompasses polynucleotides, vectors and expression vectors encoding the antigen binding protein, antibody or binding fragments thereof described herein.

The invention also relates to polynucleotides that encode any antigen binding protein or fragment as described herein. The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, genomic DNA, recombinant polynucleotides, plasmids, vectors, isolated DNA of any sequence, isolated RNA of any sequence, nucleic acid probes, and primers. A polynucleotide may be provided in isolated or purified form.

A nucleic acid sequence which “encodes” a selected polypeptide is a nucleic acid molecule, which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of this disclosure, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence. In one embodiment, a polynucleotide comprises a sequence, which encodes a VH or VL amino acid sequence as described above. The polynucleotide may encode the VH or VL sequence of a specific antigen binding protein, antibody or binding fragment thereof as disclosed herein.

An antigen binding protein, antibody or binding fragment thereof may thus be produced from or delivered in the form of a polynucleotide, which encodes, and is capable of expressing it. Where the antigen binding protein or antibody comprises two or more chains, a polynucleotide may encode one or more antibody chains. For example, a polynucleotide may encode an antibody light chain, an antibody heavy chain or both. Two polynucleotides may be provided, one of which encodes an antibody light chain and the other of which encodes the corresponding antibody heavy chain. Such a polynucleotide or pair of polynucleotides may be expressed together such that an antibody is generated.

Polynucleotides can be synthesised according to methods well known in the art, as described by way of example in Sambrook J et al. (1989, Molecular cloning: a laboratory manual; Cold Spring Harbor: New York: Cold Spring Harbor Laboratory Press).

The nucleic acid molecules of the present invention may be provided in the form of an expression cassette, which includes control sequences operably linked to the inserted sequence, thus allowing for expression of the antibody of the invention in vivo. These expression cassettes, in turn, are typically provided within vectors (e.g., plasmids or recombinant viral vectors). Such an expression cassette may be administered directly to a host subject. Alternatively, a vector comprising a polynucleotide may be administered to a host subject. Preferably the polynucleotide is prepared and/or administered using a genetic vector. A suitable vector may be any vector, which is capable of carrying a sufficient amount of genetic information, and allowing expression of a polypeptide, such as the antibody or binding fragment thereof defined above.

Also disclosed are expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals, which may be necessary, and which are positioned in the correct orientation, in order to allow for expression of a peptide of the invention. Other suitable vectors would be apparent to persons skilled in the art. By way of further example in this regard we refer to Sambrook J et al. (1989, Molecular cloning: a laboratory manual; Cold Spring Harbor: New York: Cold Spring Harbor Laboratory Press).

A person skilled in the art may use the sequences described herein to clone or generate cDNA or genomic sequences for instance such as described in the below examples. Cloning of these sequences in an appropriate eukaryotic expression vector, like pcDNA3 (Invitrogen), or derivates thereof, and subsequent transfection of mammalian cells (like CHO cells) with combinations of the appropriate light and heavy chain-containing vectors will result in the expression and secretion of the antibodies described herein.

The skilled person may also make analogues of the antigen binding proteins, antibodies or binding fragments thereof as described herein by using the specific binding domains of the antigen binding protein or antibody sequences and express them in a different context, such as a polypeptide, such as a fusion protein. This is well known in the art.

Also disclosed are cells that have been modified to express an antigen binding protein or antibody. Such cells include transient, or preferably stable higher eukaryotic cell lines, such as mammalian cells or insect cells, lower eukaryotic cells, such as yeast or prokaryotic cells, such as bacterial cells. Particular examples of cells, which may be modified by insertion of vectors or expression cassettes encoding for an antigen binding protein or antibody of the invention, include mammalian HEK293, CHO, HeLa, NS0 and COS cells. Preferably the cell line selected will be one which is not only stable, but also allows for mature glycosylation.

Such cell lines may be cultured using routine methods to produce an antigen binding protein, antibody or binding fragment thereof, or may be used therapeutically or prophylactically to deliver antigen binding proteins, antibodies or binding fragments thereof to a subject. Alternatively, polynucleotides, expression cassettes or vectors of the invention may be administered to a cell from a subject ex vivo and the cell then returned to the body of the subject.

### Pharmaceutical Compositions

When used in the methods of the invention, the therapeutic agent, preferably the antigen binding protein as defined above, may be provided as a pharmaceutical composition comprising the therapeutic agent or antigen binding protein. The invention therefore encompasses pharmaceutical compositions comprising the therapeutic agent or antigen binding protein and a pharmaceutically acceptable carrier, for use in the methods of the invention.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intraocular, intramuscular, subcutaneous, intradermal or intraperitoneal administration (e.g. by injection or infusion). In certain embodiments, a pharmaceutically acceptable carrier comprises at least one carrier selected from the group consisting of a co-solvent solution, liposomes, micelles, liquid crystals, nanocrystals, nanoparticles, emulsions, microparticles, microspheres, nanospheres, nanocapsules, polymers or polymeric carriers, surfactants, suspending agents, complexing agents such as cyclodextrins or adsorbing molecules such as albumin, surface active particles, and chelating agents. In further embodiments, a polysaccharide comprises hyaluronic acid and derivatives thereof, dextran and derivatives thereof, cellulose and derivatives thereof (e.g. methylcellulose, hydroxy-propylcellulose, hydroxy-propylmethylcellulose, carboxymethylcellulose, cellulose acetate phthalate, cellulose acetate succinate, cellulose acetate butyrate, hydroxypropylmethyl-cellulose phthalate), chitosan and derivative thereof, [beta]-glucan, arabinoxylans, carrageenans, pectin, glycogen, fucoidan, chondrotin, dermatan, heparan, heparin, pentosan, keratan, alginate, cyclodextrins, and salts and derivatives, including esters and sulfates, thereof.

Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols, such as mannitol, sorbitol, or sodium chloride in the composition.

A pharmaceutical composition may include a pharmaceutically acceptable anti-oxidant. These compositions may also contain adjuvants, such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents, which delay absorption such as aluminium monostearate and gelatin.

Therapeutic compositions typically must be sterile and stable under the conditions of manufacture and storage. The pharmaceutical composition can be formulated as a solution, microemulsion, liposome, or other ordered structure suitable to high drug concentration.

Sterile injectable solutions can be prepared by incorporating the active agent (e.g. antibody) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Pharmaceutical compositions may comprise additional active ingredients as well as an therapeutic agent as defined above. As mentioned above, compositions of the invention may comprise one or more antigen binding proteins. They may also comprise additional therapeutic or prophylactic active agents.

Depending on the route of administration, the antigen binding protein, antibody or binding fragment thereof may be coated in a material to protect the antigen binding protein or antibody from the action of acids and other natural conditions that may inactivate or denature the antigen binding protein or antibody.

In a preferred embodiment, the pharmaceutical composition according to the invention is in a form selected from the group consisting of an aqueous solution, a gel, a hydrogel, a film, a paste, a cream, a spray, an ointment, or a wrap.

In further embodiments, the pharmaceutical compositions described herein can be administered by a route such as intravenous, subcutaneous, intraocular, intramuscular, intra-articular, intradermal, intraperitoneal, spinal or by other parenteral routes of administration, for example by injection or infusion. Administration may be rectal, oral, ocular, topical, epidermal or by the mucosal route. Administration may be local, including by inhalation. In a preferred embodiment, the pharmaceutical composition is administered intravenously or subcutaneously. In one embodiment, the pharmaceutical composition may be administered by inhalation. In one embodiment, a metered dose device comprising the pharmaceutical composition is used.

Also disclosed herein are kits comprising the therapeutic agent or other compositions of the invention and instructions for use. The kit may further contain one or more additional reagents, such as an additional therapeutic or prophylactic agent as discussed herein.

### Methods for the Prevention and Treatment of Disease

The methods of the invention may be for the prevention or treatment of a disease or condition in a subject, in which case the method comprises administering said therapeutic agent, such as the antigen binding protein, to the subject in a prophylactically or therapeutically effective amount.

In therapeutic applications, the therapeutic agent, such as the antigen binding protein, are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. Effective amounts for a given purpose will depend on the severity of the disease or injury as well as the weight and general state of the subject. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. As used herein, the term “subject” includes any vertebrate, typically any mammal, such as human or horse. The subject is preferably human.

In particular embodiments, the therapeutic agent, such as the antigen binding protein, may be linked (directly or indirectly) to another moiety. The other moiety may be a further therapeutic agent such as a drug. The other moiety may be a detectable label. The other moiety may be a binding moiety, such as an antibody or a polypeptide binding domain specific for a therapeutic target. The antigen binding protein of the invention may be a bispecific antibody.

The further therapeutic agent or a detectable label may be directly attached, for example by chemical conjugation, to an antigen binding protein of the invention. Methods of conjugating agents or labels to an antigen binding protein are known in the art. For example, carbodiimide conjugation (Bauminger S and Wilchek M, 1980, Methods Enzymol., 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety.

Other methods for conjugating a moiety to therapeutic agents can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antigen binding protein or antibody maintains its targeting ability and that the functional moiety maintains its relevant function.

The further therapeutic agent linked to the antigen binding protein may comprise a polypeptide or a polynucleotide encoding a polypeptide which is of therapeutic benefit. Examples of such polypeptides include anti-proliferative or anti-inflammatory cytokines.

The antigen binding protein may be linked to a detectable label. By “detectable label” it is meant that the antigen binding protein is linked to a moiety which, when located at the target site following administration of the antigen binding protein into a patient, may be detected, typically non-invasively from outside the body and the site of the target located.

Typically, the label is or comprises a radioactive atom which is useful in imaging. Suitable radioactive atoms include 99mTc and 123I for scintigraphic studies. Other labels include, for example, spin labels for magnetic resonance imaging (MRI) such as 123I again, 131I, 111In, 19F, 13C, 15N, 170, gadolinium, manganese or iron. Clearly, the sufficient amount of the appropriate atomic isotopes must be linked to the antibody in order for the molecule to be readily detectable.

The radio- or other labels may be incorporated in known ways. For example, the antigen binding protein, antibody, or fragment thereof, may be biosynthesised or may be synthesised by chemical amino acid synthesis using suitable amino acid precursors involving, for example, fluorine-19 in place of hydrogen. Labels such as 99mTc, 123I, 186Rh, 188Rh and 111In can, for example, be attached via cysteine residues in polypeptides. Yttrium-90 can be attached via a lysine residue. Preferably, the detectable label comprises a radioactive atom, such as, for example technetium-99m or iodine-123. Alternatively, the detectable label may be selected from the group comprising: iodine-123; iodine-131; indium-111; fluorine-19; carbon-13; nitrogen-15; oxygen-17; gadolinium; manganese; iron.

The therapeutic agent of the invention, preferably the antigen binding protein of the invention, may be administered via one or more routes of administration using one or more of a variety of methods known in the art. As will be appreciated by the skilled artisan, the route and/or mode of administration will vary depending upon the desired results. Preferred routes of administration for antigen binding proteins, antibodies or compositions of the invention include intravenous, subcutaneous, intraocular, intramuscular, intradermal, intraperitoneal, spinal or other parenteral routes of administration, for example by injection or infusion. The phrase “parenteral administration” as used herein means modes of administration other than enteral and topical administration, usually by injection. Administration may be rectal, oral, ocular, topical, epidermal or by the mucosal route. Administration may be local, including peritumoral, juxtatumoral, intratumoral, to the resection margin of tumors, intralesional, perilesional, by intra cavity infusion, intravesicle administration, or by inhalation. In a preferred embodiment, the pharmaceutical composition is administered intravenously or subcutaneously.

A suitable dosage of the therapeutic agent may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular therapeutic agent employed, the route of administration, the time of administration, the rate of excretion of the therapeutic agent the duration of the treatment, other drugs, compounds and/or materials used in combination with the particular compositions employed, the age, sex, weight, condition, general health and prior medical history of the patient being treated, and like factors well known in the medical arts.

A suitable dose of an antigen binding protein may be, for example, in the range of from about 0.1 μg/kg to about 100 mg/kg body weight of the patient to be treated. For example, a suitable dosage may be from about 1 μg/kg to about 50 mg/kg body weight per week, from about 100 μg/kg to about 25 mg/kg body weight per week or from about 10 μg/kg to about 12.5 mg/kg body weight per week.

A suitable dosage may be from about 1 μg/kg to about 50 mg/kg body weight per day, from about 100 μg/kg to about 25 mg/kg body weight per day or from about 10 μg/kg to about 12.5 mg/kg body weight per day.

Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Antigen binding proteins may be administered in a single dose or in multiple doses. The multiple doses may be administered via the same or different routes and to the same or different locations. Alternatively, antigen binding proteins can be administered as a sustained release formulation, in which case less frequent administration is required. Dosage and frequency may vary depending on the half-life of the antigen binding protein in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease.

Combined administration of two or more agents may be achieved in a number of different ways. In one embodiment, the therapeutic agent of the invention and the other agent may be administered together in a single composition. In another embodiment, the therapeutic agent and the other agent may be administered in separate compositions as part of a combined therapy. For example, the therapeutic agent may be administered before, after or concurrently with the other agent.

### Diseases to be Diagnosed, Treated or Prevented

A therapeutically effective amount of the antigen binding protein (or the nucleic acid encoding the antigen binding protein) will depend upon the severity of the disease and the general state of the patient's health. A therapeutically effective amount of the antigen binding protein can provide either subjective relief of a symptom(s) or an objectively identifiable improvement as noted by the clinician or other qualified observer. As noted above, these compositions can be administered in conjunction with another therapeutic agent, either simultaneously or sequentially.

When the therapeutic activity of the therapeutic agent, such as the antigen binding protein, remains within a defined range, the therapeutic agent will retain its full therapeutic potential, while when the therapeutic activity is such that it can lead to adverse events, the modification in activity of endogenous factor will lead to cleavage of the self-regulation elements and ultimately inactivates the therapeutic agent to reduce the risk of adverse events progression.

Single or multiple administrations of the compositions including the antigen binding protein, that are disclosed herein, are administered depending on the dosage and frequency as required and tolerated by the patient. In any event, the composition should provide a sufficient quantity of at least one of the antigen binding protein disclosed herein to effectively treat the patient. The dosage can be administered once but may be applied periodically until either a therapeutic result is achieved or until side effects warrant discontinuation of therapy. In one example, a dose of the antigen binding protein is infused for thirty minutes every other day. In this example, about one to about ten doses can be administered, such as three or six doses can be administered every other day. In a further example, a continuous infusion is administered for about five to about ten days. The subject can be treated at regular intervals, such as monthly, until a desired therapeutic result is achieved. Generally, the dose is sufficient to treat or ameliorate symptoms or signs of disease without producing unacceptable toxicity to the patient.

Compositions are further disclosed that include the antigen binding protein or nucleic acid encoding the antigen binding protein in a carrier. The compositions can be prepared in unit dosage forms for administration to a subject. The amount and timing of administration are at the discretion of the treating physician to achieve the desired purposes. The antigen binding protein and/or nucleic acid can be formulated for systemic or local administration. In one example, the antigen binding protein or nucleic acid encoding the antigen binding protein is formulated for parenteral administration, such as intravenous administration. In some embodiments, administration is intramuscular.

Active ingredients can also be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Specifically, liposomes containing the antibodies can be prepared by such methods as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556. The reverse-phase evaporation method can be used with a lipid composition comprising phosphatidylcholine, cholesterol and PEG-derivatized phosphatidylethanolamine (PEG-PE). Liposomes are extruded through filters of defined pore size to yield liposomes with the desired diameter. Polypeptides of the present invention can be conjugated to the liposomes as described, for example, in Martin et al., J. Biol. Chem., 257:286-288 (1982) via a disulfide interchange reaction.

The compositions for administration can include a solution of the antigen binding protein dissolved in a pharmaceutically acceptable carrier, such as an aqueous carrier. A variety of aqueous carriers can be used, for example, buffered saline and the like. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antigen binding protein in these formulations can vary widely, and will be selected primarily based on fluid volumes, viscosities, body weight and the like in accordance with the particular mode of administration selected and the subject's needs. In some embodiments, administration is intravenous.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the antigen binding protein disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm.112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)).

A typical pharmaceutical composition for intravenous administration includes about 0.1 to 10 mg/kg of antigen binding protein per day, or 0.5 to 15 mg/kg of antigen binding protein per day. Dosages from 0.1 up to about 100 mg/kg per subject per day may be used, particularly if the agent is administered to a secluded site and not into the circulatory or lymph system, such as into a body cavity or into a lumen of an organ. Exemplary doses include 1 to 10 mg/kg, such as 2 to 8 mg/kg, such as 3 to 6 mg/kg. Actual methods for preparing administrable compositions will be known or apparent to those skilled in the art and are described in more detail in such publications as Remington's Pharmaceutical Science, 19th ed., Mack Publishing Company, Easton, PA (1995).

Antigen binding proteins may be provided in lyophilized form and rehydrated with sterile water before administration, although they are also provided in sterile solutions of known concentration. The antigen binding protein solution is then added to an infusion bag containing 0.9% sodium chloride, USP, and typically administered at a dosage of from 0.1 to 10 mg/kg or 0.5 to 15 mg/kg of body weight. Exemplary doses include 1 to 10 mg/kg, such as 2 to 8 mg/kg, such as 3 to 6 mg/kg. Considerable experience is available in the art in the administration of antibody drugs, which have been marketed in the U.S. since the approval of RITUXAN® in 1997. Antigen binding proteins can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

A therapeutically effective amount of a nucleic acid encoding the antigen binding protein can be administered to a subject in need thereof. One approach to administration of nucleic acids is direct immunization with plasmid DNA, such as with a mammalian expression plasmid. The nucleotide sequence encoding the antigen binding protein can be placed under the control of a promoter to increase expression of the molecule. Immunization by nucleic acid constructs is well known in the art and taught, for example, in U.S. Pat. Nos. 5,643,578, and 5,593,972 and 5,817,637. U.S. Pat. No. 5,880,103 describes several methods of delivery of nucleic acids to an organism. The methods include liposomal delivery of the nucleic acids.

In another approach to using nucleic acids, an antigen binding protein can also be expressed by attenuated viral hosts or vectors or bacterial vectors, which can be administered to a subject. Recombinant vaccinia virus, adeno-associated virus (AAV), herpes virus, retrovirus, cytomegalovirus, poxvirus or other viral vectors can be used to express the antibody. For example, vaccinia vectors are described in U.S. Pat. No. 4,722,848. BCG (Bacillus Calmette Guerin) provides another vector for expression of the disclosed antigen binding proteins (see Stover, Nature 351:456-460, 1991).

In one embodiment, a nucleic acid encoding the antigen binding protein is introduced directly into cells. For example, the nucleic acid can be loaded onto gold microspheres by standard methods and introduced into the skin by a device such as Bio-Rad's Heliosä Gene Gun. The nucleic acids can be “naked,” consisting of plasmids under control of a strong promoter.

Typically, the DNA is injected into muscle, although it can also be injected directly into other sites. Dosages for injection are usually around 0.5 mg/kg to about 50 mg/kg, and typically are about 0.005 mg/kg to about 5 mg/kg (see, e.g., U.S. Pat. No. 5,589,466).

In some examples, a subject is administered the DNA encoding the antigen binding protein to provide in vivo antigen binding protein production, for example using the cellular machinery of the subject. Immunization by nucleic acid constructs is well known in the art and taught, for example, in U.S. Pat. Nos. 5,643,578, and 5,593,972 and 5,817,637. U.S. Pat. No. 5,880,103 describes several methods of delivery of nucleic acids encoding to an organism. The methods include liposomal delivery of the nucleic acids. Such methods can be applied to the production of an antigen binding protein, by one of ordinary skill in the art.

One approach to administration of nucleic acids is direct administration with plasmid DNA, such as with a mammalian expression plasmid. The nucleotide sequence encoding the disclosed antibody can be placed under the control of a promoter to increase expression.

### Treatment of Hemophilia A

Embodiments of the invention where the self-regulation element comprises a Thrombin, Factor IXa, Factor Xa, Factor Xia, Factor XIIIa or APC cleavage site are useful for the treatment of hemophilia A. The embodiments of the invention where the self-regulation element comprises a Thrombin cleavage site are useful for use in the treatment of hemophilia A. In a preferred embodiment of the invention, the antigen binding protein of the invention useful for the treatment of hemophilia A comprises a Thrombin cleavage site. In a preferred embodiment of the invention, the antigen binding protein of the invention useful for the treatment of hemophilia A comprises an self-regulation element as set out in SEQ ID NO: 1, 2, 3 or 4. In a preferred embodiment of the invention, the antigen binding protein of the invention useful for the treatment of hemophilia A comprises an self-regulation element as set out in SEQ ID NO: 1.

In a preferred embodiment of the invention, the antigen binding protein comprising the Thrombin cleavage site comprises a first antigen binding domain that specifically binds to FIXa/FIX, as defined herein, and a second antigen binding domain that specifically binds to FX/FXa, as defined herein, and a Thrombin cleavage site as defined herein.

Thus, a method for treating hemophilia A in a subject is also disclosed, the method comprising administering to the subject a therapeutically effective amount of the disclosed antigen binding protein and/or a nucleic acid encoding the antigen binding protein, thereby treating hemophilia.

The present invention also relates to the disclosed antigen binding protein for use in the treatment of hemophilia A. Further, the present invention also relates to use of the disclosed antigen binding protein in the manufacture of a medicament for the treatment of hemophilia A.

Embodiments of the invention where the self-regulation element comprises a Thrombin, Factor IXa, Factor Xa, Factor Xia, Factor XIIIa or APC cleavage site are useful for the prevention of pro-thrombotic risks. The embodiments of the invention where the self-regulation element comprises a Thrombin cleavage site are useful for use in the prevention of pro-thrombotic risks. In a preferred embodiment of the invention, the antigen binding protein of the invention useful for the prevention of pro-thrombotic risks comprises a Thrombin cleavage site. In a preferred embodiment of the invention, the antigen binding protein of the invention useful for the prevention of pro-thrombotic risks A comprises a self-regulation element as set out in SEQ ID NO: 1, 2, 3 or 4. In a preferred embodiment of the invention, the antigen binding protein of the invention useful for the prevention of pro-thrombotic risks comprises a self-regulation element as set out in SEQ ID NO: 1.

Thus, in another embodiment of the invention, a method for preventing pro-thrombotic risks in a subject is also disclosed, the method comprising administering to the subject a therapeutically effective amount of the disclosed antigen binding protein and/or a nucleic acid encoding the antigen binding protein, thereby preventing pro-thrombotic risks.

The present invention also relates to the disclosed antigen binding protein for use in the prevention of pro-thrombotic risks. Further, the present invention also relates to use of the disclosed antigen binding protein in the manufacture of a medicament for the prevention of pro-thrombotic risks.

### Treatment of Cancer

Embodiments of the invention where the self-regulation element comprises a Granzyme A, Granzyme B, Granzyme K, Granzyme H, Granzyme M, Cathepsin B, Cathepsin L, Cathepsin W, or matrix metalloprotease such as MMP-2, MMP-9 and MMP-28, cleavage site are suitable for use in the treatment of cancer. The embodiments of the invention where the self-regulation element comprises a Granzyme B cleavage site are suitable for use in the treatment of cancer. In a preferred embodiment of the invention, the antigen binding protein comprises a self-regulation element as set out in any one of SEQ ID NOs: 5 to 18. In a preferred embodiment of the invention, the antigen binding protein comprises a self-regulation element as set out in SEQ ID NO: 7. In a preferred embodiment of the invention, the antigen binding protein comprising the Granzyme B cleavage site comprises a first antigen binding domain that specifically binds to ROR1, as defined herein. In a preferred embodiment of the invention, the antigen binding protein comprising the Granzyme B cleavage site comprises a first antigen binding domain that specifically binds to ROR1, as defined herein, and a second antigen binding domain that specifically binds to CD3, as defined herein.

Thus, a method for treating cancer in a subject is also disclosed, the method comprising administering to the subject a therapeutically effective amount of the disclosed antigen binding protein and/or a nucleic acid encoding the antigen binding protein, thereby treating cancer.

The disclosed antigen binding proteins can be cytotoxic to cancer cells.

Preferably, the cancer is leukaemia (such as Chronic Lymphocytic Leukaemia (CLL), Acute Lymphoblastic Leukaemia (ALL), Mantle Cell Leukaemia or Hairy Cell Leukaemia), pancreatic cancer, prostate cancer, colon cancer, bladder cancer, ovarian cancer, glioblastoma, testicular cancer, uterine cancer, adrenal cancer, breast cancer, lung cancer, melanoma, neuroblastoma, sarcoma, renal cancer. Furthermore, ROR1 is expressed on a subset of cancer stem cells.

The present invention also relates to the disclosed antigen binding protein for use in the treatment of cancer. Further, the present invention also relates to use of the disclosed antigen binding protein in the manufacture of a medicament for the treatment of cancer.

Preferably, the cancer is leukaemia (such as Chronic Lymphocytic Leukaemia (CLL), Acute Lymphoblastic Leukaemia (ALL), Mantle Cell Leukaemia or Hairy Cell Leukaemia), pancreatic cancer, prostate cancer, colon cancer, bladder cancer, ovarian cancer, glioblastoma, testicular cancer, uterine cancer, adrenal cancer, breast cancer, lung cancer, melanoma, neuroblastoma, sarcoma, renal cancer. Furthermore, ROR1 is expressed on a subset of cancer stem cells.

The cancer or tumour does not need to be completely eliminated for the composition to be effective. For example, the antibody can reduce the tumour by a desired amount, for example by at least 10%, at least 20%, at least 50%, at least 60%, at least 70%, at least 80%, at least 90%, at least 95%, at least 98%, or 100%, as compared to the absence of the composition.

Administration of the antigen binding protein of the present invention may result in a 5, 10, 20, 50, 75, 90, 95 or 99% depletion, i.e. reduction in malignant cells.

In another example, the subject can also be administered an effective amount of an additional agent, such as a chemotherapy agent. The methods can include administration of one or more additional agents known in the art.

Severe toxicities associated with the mode-of-action (MoA) of immunotherapies include (but are not restricted to) cytokine release syndrome (CRS) and neurotoxicity, and result in an acute systemic inflammatory syndrome characterized by fever and multiple organ dysfunction. Acute systemic inflammatory syndromes can be associated with i) chimeric antigen receptor (CAR)-T cell therapy, (ii) therapeutic antibodies, and (iii) haploidentical allogeneic stem cell transplantation (haplo-allo-HSCT). Granzyme B levels are increased in subjects progressing toward severe MoA-related toxicities. Embodiments of the invention where the self-regulation element comprises a Granzyme A, Granzyme B, Granzyme K, Granzyme H, Granzyme M, Cathepsin B, Cathepsin L, Cathepsin W, or matrix metalloprotease such as MMP-2, MMP-9 and MMP-28, cleavage site are suitable for use in the prevention of MoA-related toxicities associated with immunotherapies, such as cytokine release syndrome. Embodiments of the invention where the self-regulation element comprises a Granzyme B cleavage site are suitable for use in the prevention of MoA-related toxicities associated with immunotherapies. In a preferred embodiment of the invention, the antigen binding protein comprises a self-regulation element as set out in any one of SEQ ID NOs: 5 to 18. In a preferred embodiment of the invention, the antigen binding protein comprises a self-regulation element as set out in SEQ ID NO: 7.

In another embodiment of the invention, a method for preventing MoA-related toxicities associated with immunotherapies in a subject is also disclosed, the method comprising administering to the subject a therapeutically effective amount of the disclosed antigen binding protein and/or a nucleic acid encoding the antigen binding protein, thereby preventing MoA-related toxicities.

The present invention also relates to the disclosed antigen binding protein for use in the prevention of MoA-related toxicities associated with immunotherapies. Further, the present invention also relates to use of the disclosed antigen binding protein in the manufacture of a medicament for the prevention of MoA-related toxicities.

Other antigen binding proteins that may benefit from introduction of a Granzyme B self-regulation element in order to prevent MoA-related toxicities include moruomab, basiliximab, daclizumab, blinatumumab and alemtuzumab.

In another embodiment of the invention, a method for preventing cytokine release syndrome in a subject is also disclosed, the method comprising administering to the subject a therapeutically effective amount of the disclosed antigen binding protein and/or a nucleic acid encoding the antigen binding protein, thereby preventing cytokine release syndrome.

The present invention also relates to the disclosed antigen binding protein for use in the prevention of cytokine release syndrome. Further, the present invention also relates to use of the disclosed antigen binding protein in the manufacture of a medicament for the prevention of cytokine release syndrome.

Other antigen binding proteins that may benefit from introduction of a Granzyme B self-regulation element in order to prevent cytokine release syndrome include moruomab, basiliximab, daclizumab, blinatumumab and alemtuzumab.

### EXAMPLES

### Example 1—Rationale

### FVIII Mimetic Antibodies in Haemophilia A

Coagulation Factor VIII (FVIII) is a critical cofactor in the coagulation cascade, promoting the catalytic conversion of Factor X (FX) into activated FX (FXa) by forming a complex with activated Factor IX (FIXa) and FX. In Haemophilia A (an X-linked monogenic disorder with a prevalence of 1 in 5,000 male births) a deficiency in FVIII disrupts coagulation, leading to life-threatening, spontaneous bleeding diathesis. FVIII replacement, the standard of care treatment, requires frequent intravenous infusions of FVIII. However approximately 30% of treated patients develop anti-FVIII neutralizing antibodies, rendering FVIII replacement inefficient and significantly reducing life expectancy.

Emicizumab, a humanised IgG4 bispecific antibody approved in late 2017, is a FVIII mimetic molecule that works by simultaneously binding FIXa and FX to promote the catalytic conversion of FX into activated FXa via FIXa, leading to thrombin generation and the restoration of clot formation. With 80% reduction in bleeding episodes and prophylaxis achievable even in patients with neutralizing antibodies, emicizumab has redefined the treatment of haemophilia A. However, an abnormally high incidence of life-threatening thrombotic microangiopathies and thromboembolisms (3 cases per 1000 treated patients) as well as unexplained fatalities has been observed in patients treated with emicizumab and has led the FDA to issue a black-box warning to the use of this therapy.

The mechanism of action by which FVIII and emicizumab sustain the coagulation cascade differs profoundly (Lenting, Denis et al. 2017). FVIII circulates in a non-active form and requires activation into FVIIIa to promote amplification of thrombin formation. Thrombin further activates Protein C (PC) to inhibit FVIIIa through a negative feedback-loop that prevents excessive procoagulant activity. In contrast, emicizumab is in a permanently active state that cannot be “switched-off” by activated PC (aPC) after clot formation and therefore promotes a persistent prothrombotic state for as long as it remains in circulation. This dangerous state could be overcome by the development of an autoregulated FVIII mimetic antibody. Introducing a thrombin cleavable peptide to act as a built-in “switch” mechanism will enable the autoregulated antibody to be cleaved into non-functional elements when sufficient thrombin has been generated to form the clot, thereby restoring the negative feedback loop and preventing the risk of thrombosis.

### Bispecific T Cell Engager in Adoptive Immunotherapy

Bispecific T cell engagers (TCEs) are bispecific antibodies that redirect the cytotoxic activity of T cells towards malignant tumour cells. This is an extremely effective mechanism of action, however it is also associated with the development of severe adverse events resulting in life-threatening immune hyperactivation including cytokine release syndrome (CRS) and neurological toxicities in a substantial number of patients. Indeed, a 100% rate of adverse events have been observed with the therapeutic use of TCEs, with grade 3 and 4 life-threatening toxicities effecting over 50% of patients treated with the currently approved TCEs (blinatumomab, mosenutuzumab and teclistamab). Mitigating measures such as reducing tumour load, corticosteroid administration and gradual dose escalation help but do not eliminate these toxicities. These limitation constrain optimal dosing, shorten treatment duration, and increase the burden of monitoring, particularly for complex or vulnerable patients. Therefore, the development of an autoregulated TCE (AR_TCE) that inactivates in response to biological cues in a threshold limited manner and prior to grade 3/4 life threatening toxicity would efficiently reduce the occurrence of adverse events and broaden the therapeutic index for TCEs resulting in improved outcome for cancer patients.

Upon engagement of both targets, TCEs cluster CD3 at the surface of T cells resulting in activation and immune synapse formation. This stimulates the local release of cytotoxic enzymatic factors, including granzyme B, which triggers apoptosis and elimination of the target cell. The efficient killing of cancer cells requires this T cell mediated cytokine and proteolytic enzyme release, which at low levels is associated with the mode-of-action of TCEs. However, experience from TCE and CAR-T cell studies showed that life-threatening MoA-related toxicities are the hallmark of an overdrive in T cell activation, in which granzyme B levels can be increased by up to 40-fold (Kochenderfer, Somerville et al. 2017). Therefore, insertion of a granzyme B sensitive peptide in between the targeting and effector arm of a TCE will render the drug susceptible to degradation and inactivation upon exposure to rising levels of granzyme B above a threshold where it is associated with the development of MoA-related toxicities and thus reduce the risk of severe adverse events.

### Chimeric Antigen Receptor (CAR)-T Cell Therapies

Chimeric antigen receptor (CAR)-T cell are cellular therapies genetically engineered to express a chimeric cell surface receptor targeting a tumour associated antigen (TAA) of interest, and have proven clinically effective at redirecting T cell cytotoxicity to eliminate cancer cells. The mode-of-action of CAR-T cell is comparable to that of bispecific TCEs and, as such, are associated with high risks of severe MoA-related toxicities characteristic of T cell redirecting technologies including cytokine release syndrome and neurological toxicities.

The hallmarks of CAR-T cell toxicities are similar to those of TCEs and include granzyme B release as a consequence of CAR-T cell overactivation. Therefore, the development of an autoregulated AR_CAR-T cell by insertion of a granzyme B sensitive peptide in the structure of the CAR receptor would enable the cleavage and removal of the receptor for the cell surface in response to cues associated with the progression of MoA-related toxicities and would therefore significantly improve the safety profile of this class of therapeutics without affecting its potency.

### Example 2—Materials and Methods

Production and purification of bispecific antibodies Emicizumab was obtained from left-over vials of the commercial Hemlibra™ provided by the Katharine Dormandy Haemophilia and Thrombosis center (Royal Free hospital, London). Production and purification of the autoregulated prototype AR_Ab8 was outsourced to Absolute Antibody (now Absolute Biotech). The antibody was expressed in the human embryonic kidney (HEK)-293 cell line and purified using Absolute Antibody's proprietary method with a final product showing >99% purity by size exclusion chromatography.

NVG-111 and autoregulated AR_TCE variants were produced in-house using transient transfections in the mammalian Expi293 expression system (Gibco). High-density Expi293F™ cells were sub-cultured routinely at 37° C., 8% CO₂ and 130 rpm in Expi293 expression medium. For transfections, cells were seeded the day before at 1×10⁶ viable cells per ml in Expi293 expression medium. On the following day, desired volumes of cell suspensions were transfected with plasmids containing coding sequences of the different antibodies cloned in the pcDNA3.1hygro(+) expression vector, using ExpiFectamine™ according to the manufacturer's instructions. 16 to 18 h post-transfection, cells were fed with production enhancers as per recommendations to increase protein yields, and cultures were incubated for an additional 5 days. Production supernatants were harvested clarified by both centrifugation (4000 g for 20 min) to remove the cells and filtration through a 0.22 μm filter. The different variants were purified by affinity chromatography using 5 mL HiTrap™ columns with the MabSelect™ PrismA resin (Cytiva) on an ÄKTA avant 25 system (Cytiva). Purified proteins were eluted in 50 mM glycine, 150 mM NaCl, pH3 and immediately neutralized with 2% v/v of a 2M Tris-Base solution, pH8.3. Post-purification, the antibodies were buffer exchanged into a formulation solution containing 10 mM histidine, 150 mM NaCl, 0.02% Tween80 at pH6.0 and stored at −80° C.

### Chromogenic Assay

The FVIII mimetic activity of emicizumab and AR_Ab8 was evaluated by measuring the FIX-mediated activation of FX into FXa, using the FXa specific S-2765 chromogenic substrate. In a polystyrene 96 flat-bottom half-well plate (Greiner Bio-one), 50 uL of a coagulation factor solution containing 40 uM phospholipid-TGT (Rossix AB), 280 nM of plasma-derived human FX (Coagadex®, Bio Product Laboratory), 6 nM of plasma-derived human FIXa (Haematologic Technologies) and 10 mM CaCl₂) in running buffer (50 mM Tris-Base, 150 nM NaCl, 0.1% BSA-protease free, pH 7.8) was mixed with 25 uL of purified antibody at 2 nM and incubated 15 min at 37° C. 25 uL of S-2765 chromogenic substrate (Chromogenix) diluted at 1.2 mM in running buffer was then added to each well and the kinetic of chromogenic conversion was immediately recorded at 405 nm on a plate reader (SpectraMax m3, Molecular Devices) for 20 min at 37° C. (1 read/minute, 2 sec shake before each read).

Activated Partial Thromboplastin Time (aPTT)

A citrated pool of human plasma made from at least 20 healthy volunteers was spiked with 200 Bethesda Units of a FVIII-neutralising polyclonal antibody (PAHFVIII-S, Haematologica Technologies) to removed FVIII activity. The induced haemophilia A plasma was then supplemented with 350 nM of emicizumab or AR_Ab8 and incubated for 15 min at room temperature. Clotting time was measured on a ACL Top 700 coagulometer (Werfen Limited) using the aPTT synthaSil reagents (Werfen Limited).

### Thrombin Cleavage Assay

To evaluate thrombin-mediated inactivation of the bispecific antibodies, a solution of 2 nM of emicizumab or AR_Ab8 was mixed with 2 U/mL of human α-thrombin (Enzyme Research Laboratories) in running buffer (50 mM Tris-Base, 150 nM NaCl, 0.1% BSA-protease free, pH 7.8) and incubated at 37° C. the cleavage reaction was stopped at different time-points from 15 min to 3 h by adding v/v of a solution of PPACK inhibitor (Merck—Millipore) at 500 nM and samples were frozen for further analysis.

Antibody cleavage was detected by SDS-Page and Western blotting. Samples were loaded undiluted in a NuPAGE 4-12% bis-Tris precast gel (Invitrogen) and migration was performed between 120-180V in MOPS-SDS running buffer (Invitrogen). 5 uL of prestained PageRuler Protein Ladder (Thermofisher) was added in a separated well for molecular weight comparison. Proteins were further transferred in a nitrocellulose blotting membrane using a wet-transfer method in NuPAGE transfer buffer (Invitrogen). The membrane was saturated for 1 h at room temperature in TBS.T buffer (50 mM Tris-Base, 150 mM NaCl, 0.1% Tween20, pH 7.8) with 5% skimmed milk on an orbital shaker, and further blotted with a Horse Radish Peroxidase (HRP)-labelled polyclonal goat anti-human Fc antibody (#31413, Invitrogen) diluted 1:5000 in TBS.T+5% milk, overnight at 4° C. Membranes were washed 3 times in TBS.T and incubated 5 min with SuperSignal West Pico PLUS chemiluminescent substrate (Thermofisher) at room temperature. Images were acquired on GelDoc system (Bio-Rad).

Residual FVIII mimetic activity of the samples was measured using the chromogenic assay as previously described. Samples were diluted 1:2 to bring the bispecific antibody concentration to 1 nM.

### Prothrombotic Thrombin Generation Assay (TGA)

Abnormal thrombin generation was measured with the calibrated automated thrombogram (CAT) method on a thrombinoscope (Diagnostica stago). FVIII activity was neutralized as previously described in a pool of human plasma with 200 BU of anti-FVIII antibody. The neutralised plasma was then supplemented with 600 nM of emicizumab or AR_Ab8 in combination with 0.5 U/mL of aPCC (FEIBA, Takeda Pharmaceutical) and incubated for 15 min at room temperature. For each condition, 80 uL of plasma was mixed with 20 uL of the Tissue Factor PPPlow trigger (Diagnostica stago) in an Immulon 2HB U-bottom plate (Diagnostica Stago) and incubated 10 min at 37° C. Thrombin generation reaction was initiated by the automated injection of 20 uL of pre-warmed FluCa reagent and data were recorded for 60 min. Endogenous Thrombin Potential (ETP) is calculated as the area under the thrombogram curve and represents the total thrombin production.

### Tail Clip Assay

The tail clip assay was used to evaluated the procoagulant potential FVIII mimetic bispecific antibodies in FVIII deficient mice and the procedure was conducted as previously described (Ferriere et al., blood 2020). Briefly, FVIII-deficient male mice (aged 8-12 weeks) received 3 mg/kg of emicizumab or AR_Ab8 by retro-orbital injection 24 hours before the procedure. A second retro-orbital injection with a mixture of 100 U/kg of both human FIX and FX was given 5 minutes before the 3 mm distal tip of the tail was amputated under ketamine (100 mg/kg)/xylazine (10 mg/kg) anaesthesia. Control animals received a single retro-orbital injection with 2 U/mice of recombinant human FVIII. The severed tail was then immersed in prewarmed physiological saline (37° C.) and blood was collected for 30 minutes. The volume of blood loss was quantified by measuring the amount of haemoglobin in the collection tube by spectrophotometry at 416 nm and calculated from a standard curve.

### In Vivo Thrombosis Model

The thrombotic potential of FVIII mimetic bispecific antibodies was evaluated in vivo using experiment protocol aiming to reproduce in mice the combined effects of emicizumab and aPCC. C57Bl/6 male mice aged 8-10 weeks (Charles River Laboratory) received an initial intravenous (IV.) injection with 2.5 U of aPCC (FEIBA, Takeda Pharmaceutical) in combination with 250 ug of bispecific antibody, followed by 3 boosted injections of aPCC (IV., 2.5 U per injection) at 24, 48 and 72 h. Control animals only received aPCC without the bispecific antibodies. At 96 h, all animals were bled by cardiac puncture from the posterior vena cava on EDTA anticoagulant (final concentration 0.05M) and under non-recovering gaseous anaesthesia (Isoflurane). All animals were humanely euthanised by cervical dislocation. Immediately following the bleed, a platelet count was performed using an automated cell counter (Scil Vet ABC Plus, Horiba Medical).

The lungs were explanted for thrombosis analysis and fixed in 10% neutral buffered formalin (CellPath limited) for 24 h at room temperature. Fixed organs were briefly washed in PBS, embedded in OCT matrix (CellPath limited) and frozen in liquid nitrogen vapours. Tissue sections were prepared from lung cryoblocs and processed for immunofluorescent (IF) staining of platelet and blood vessel. The lung sections were washed 10 min in PBS to remove the OCT matrix and a detergent-based antigen retrieval step was performed with a 10 min incubation in PBS+0.05% Tween 20 (PBS.T) and 0.5% Triton X-100. Sections were washed 3×5 min in PBS.T and non-specific binding sites saturated with 3% Bovine Serum Albumin (BSA) in PBS.T (w/v) for 1 h at room temperature (RT). The section was incubated overnight at 4° C. with a rat anti-mouse CD41/integrin αIIb (clone MWreg30, BD bioscience) and a goat anti-mouse CD31/PECAM-1 (AF3628, Bio-Techne) diluted in PBS.T+1% BSA to detect platelets and endothelial cells respectively. The slides were washed 3×5 min in PBS.T and incubated with Alexa 488 donkey anti-rat (A48269) and Alexa-555 donkey anti-goat (A32816) polyclonal antibodies diluted in PBS.T+1% BSA for 1 h at RT on an orbital shaker. A final 3×5 min wash step was performed before the slides were mounted under coverslip with a drop of ProLong Diamond antifade reagent with DAPI (ThermoFisher Scientific) and allowed to dry for 24 h in the dark.

Images were acquired on a Z1 inverted microscope (Zeiss) with a 20× objective. To analyse thrombus formation, all individual blood vessels (identified through CD31 staining) with a section surface >10⁴ μm² were imaged in a single lung section. The CD31 staining was used to manually draw the edge of vessel using Fiji software (open source). Using the αIIb (platelet) staining channel, a threshold of signal intensity was empirically defined based on the signal over noise ratio, transformed in a binary occlusion mask to define positive and negative area of occlusion, and applied to all images with the same experiment. The percentage of occlusion for each individual blood vessel was calculated using Fiji software (Open-source software, GNU general public license) as the percentage of surface occupied by the occlusion mask within the boundaries on the blood vessel.

Protease Mediated Cleavage Assay with TCEs

Granzyme B specific cleavage of the autoregulated AR_TCE variants was evaluated in vitro. Recombinant human granzyme B protein (Bio-Techne) was first activated with mouse cathepsin C (Bio-Techne) as recommended in the manufacturer's instruction. 1 μg/mL of either NVG-111 or the AR_TCE was then mixed with various concentrations of activated granzyme B diluted in assay buffer (50 mM Tris-Base, 150 mM NaCl, 5 mM CaCl₂, 100 μg/mL protease free BSA, pH 7.6) and incubated for 2 h at 37° C. The catalytic reaction was stopped by the addition of a broad-spectrum protease inhibitor cocktail (Tebu-Bio) to a final concentration of 1× (1:10 dilution) and samples were frozen for further analysis. Antibody cleavage was visualised by SDS-Page electrophoresis and western blotting with undiluted samples and following the protocol described above. Membranes were blotted with HRP-labelled Protein L (Genscript) diluted 1:2000 in TBS.T+5% milk, overnight at 4° C.

To evaluate the sensitivity of AR_TCEs to various proteases, the cleavage assay was performed as described with a panel of representative proteases at single concentration of 50 nM. The panel was composed of activated FIIa, FVIIa and FXa (Prolytix), caspase-3, -8 (Bio-Techne) and -9 (Abcam), and the matrix-metalloprotease MMP-3 and -9 (Abcam).

The residual amount of intact antibody was quantified using a dedicated ELISA specific for intact molecules. Polystyrene 96 flat-bottom half-well plate (Greiner Bio-one) were coated with 2 μg/mL of ROR1 protein (Abcam) diluted in carbonate buffer (Na₂CO₃ 12.2 mM, NaHCO₃ 35 mM, pH9.6) overnight at 4° C. Plates were washed 3 times in TBS.T (50 mM Tris-Base, 150 mM NaCl, 0.1% Tween20, pH 7.8) and non-specific binding sites were saturated with TBS.T+3% BSA for 1 h at 37° C. Following a 3× wash step, samples were diluted in TBS.T+1% BSA and incubated for 2 h at 37° C. Unbound samples were washed 3× in TBS.T and bound intact antibodies were detected by their histidine tag motif present on the CD3 binding arm (released if cleaved), using a HRP-labelled monoclonal anti-His tag antibody (clone J099B12, Biolegend) diluted 1:2500 in TBS.T+1% BSA and incubated 2 h at 37° C. After a final 3× wash step, the plates were incubated with slow kinetic 3,3′,5,5′-Tetramethylbenzidine (TMB) substrate and colour was allow to developed for 5 to 15 min. The reaction was stopped by the v/v addition of 2M H₂SO₄ and optical density was read at 450 nm within 30 min on a SpectraMax m3 (Molecular Devices).

### T Cell Preparation and Culture

Human Peripheral Blood Mononuclear Cells (PBMCs) were obtained from leukocyte reduction chamber of healthy individuals and purified by Ficoll-Plaque density gradient. PBMCs were then cultured at 37° C. and 8% CO₂, at a density of 5×10⁵ cells/ml in RPMI1640 medium (Life Technologies) supplemented with 10% Fetal Bovin Serum (FBS, Gibco) and 100 U/mL of recombinant human Interleukin-2 (IL-2, Miltenyi Biotec) for 5 days, thus permitting the progressive elimination of non-T cell related lineage.

### Short-Term Coculture Assay

NVG-111 and AR_TCE variants were characterized in a short term coculture assay to evaluate T cell engagement and the cytotoxic potential against the ROR1+ Mantle Cell Lymphoma Jeko-1 cell line. To track target cells in the coculture, Jeko-1 cells were stained beforehand with CellTrace™ Violet (CTV; C34557; Life Technologies) according to the manufacturer's instructions. Jeko-1 cells were then mixed with an excess of purified human T cells (prepared as described above) to achieved a 1:5 target to effector cell ratio, and cocultured for 48 h in presence of increasing concentration of NVG-111 or AR_TCE. At the end of the assay, cells were spun down 5 min at 300 g and washed once in PBS. The pellet was resuspended in a solution of PBS containing a Live/Dead fixable green dye (dilution 1:800, L23101, Life Technology) and an APC-labelled anti-human CD69 antibody (dilution 1:50, 310910, Biolegend). The cells were then incubated 60 min at 4° C., washed once in PBS and analyzed by flow cytometry on CytoFlex (Beckman Coulter Life Sciences). A gating on CTV staining was used to discriminate between T cells (CTV−) and Jeko-1 cells (CTV+). Cytotoxicity was measured as the percentage of dead cells (positive staining for the Live/Dead marker) within the Jeko-1 population. T cells activation was measured by the Mean Fluorescence Intensity (MFI) in the T-cell population. All flow cytometry data were analyzed using FlowJo software (BD Biosciences).

### Long-Term Coculture Assay

The effect of autoregulation on cytokine release in vitro was evaluated in a long-term coculture assay with an excess of target cells to simulate serial T cell engagement. Purified human T cells were prepared as described above and mixed with an excess of Jeko-1 cells at a 10:1 target to effector ratio. The cells were then cocultured for up to 120 h in presence or not of 1 μg/mL of NVG-111 or the AR_TCEs. Coculture samples were harvested every 24 h and centrifugated 5 min at 300 g to separate the cells from the supernatant, which was collected and stored at −80° C. for further analysis. The cell pellet was washed once in PBS and incubated in a solution Live/Dead fixable green dye as described above. Cells were washed again in PBS and fixed for 20 min at 4° C. using the CytoFix/CytoPerm reagent (BD Biosciences).

To evaluate cytotoxic potential and T cell activation, fixed cells were probed with a Pacific Blue-labelled anti-human CD3 antibody (dilution 1:50, 300417, Biolegend) and a PE-labelled anti-human CD25 antibody (dilution 1:50,356134, Biolegend) using the protocol previously described and further analyzed by flow cytometry. T cells and target cells were discriminated using a gating strategy on CD3. Cytotoxicity was measured as the percentage of dead cells within the CD3-population (Jeko-1 cells) and T cell activation was measured as the mean MFI of CD25 expression in the T-cell population (CD3+). The release of human INFγ from activating T cells was measured in the coculture supernatant using the ELISA MAX Deluxe set (Biolegend) according to the manufacturer's recommendations. Cleavage of the AR_TCE was detected by SDS-Page electrophoresis and Protein L immunoblotting as described above using undiluted supernatant samples.

In Vivo Models of Severe Toxicities Associated with TCEs Mode-of-Action, Such as Cytokine Releases Syndrome

The potential of AR_TCEs to reduce the risk of severe toxicities related to their mode-of-action and excessive T cell engagement was tested in an in-house model of adoptive immunotherapy in mice xenografted with either a Triple Negative Breast Cancer (TBNC) solid tumour or pancreatic disseminated cancer cells, both of which are ROR1+.

NOD.SCID Gamma (NSG) male mice aged 8-10 weeks (Charles River Laboratory) were initially induced with a proprietary method to express stable circulating levels of either NVG-111 or the autoregulated AR_TCE-3 variants. For the TNBC model, 2×10⁶ cells of the MDA-MB-231 cell line were then injected subcutaneously in the mammary fat pad of the mice and the resulting solid tumour was allowed to engraft for 25 days. In the pancreatic cancer model, 2×10⁶ PANC-1 cells were directly injected in the peritoneal cavity and the mice were allowed to recover for a period of 8 days. Once the tumour was established, all animals were treated with either 6 (TNBC) or 4 (Pancreatic) cycles of purified human T cells injections. The T cells were prepared as described above, resuspended 100 μL of PBS and injection were done intravenously in the caudal vein of the mice. The first injection was done with 10⁷ T cells per mice with half this dose for the subsequent one and injections were spaced out on a 4 days/3 days schedule.

Animal weight was recorded daily during the T cell administration phase and normalised to the weight measured on the day of the first injection (day 25 for TNBC and day 8 for pancreatic cancer). The study protocol has an ethical limit of 20% weight loss from that mark any animal less than 1 g above that value was humanely culled. In addition, the general well-being of mice as well as the aspect of the solid tumour (in the TNBC model) were observed daily by an independent animal facility staff and any signs of suffering, inflammation or ulceration of the tumour was cause for ethical culling of the animals.

In the TNBC model, progression of the solid tumour was monitored every 2 to 3 days. The mean tumour diameter (d) was measured using a micrometric calliper on 2 different axis and the volume (V) was calculated using sphere formula V=(4/3)×π×(d/2)³. Tumour volumes calculated at days 15 and 18 were averaged and used as a reference to measure tumour progression. In the pancreatic cancer model, Bioluminescence imaging (BLI) was used to evaluate tumour burden form the luciferase-expressing PANC-1 cells. Mice were placed under gaseous anaesthesia and injected in the peritoneal cavity with 200 μg/mice of D-Luciferin (Melford Laboratories) diluted in 200 μL of PBS. BLI images were acquired 15 min post injection on an IVIS Lumina II in vivo imaging system (Perkin-Elmer). Images were processed with Fiji software.

Upon completion of the study or early culling, mice were bled by cardiac puncture from the posterior vena cava on EDTA anticoagulant (final concentration 0.05M) and under non-recovering gaseous anaesthesia (Isoflurane). All animals were humanely euthanised by cervical dislocation. Blood samples were centrifuged 20 min at 1500 g to separate the plasma fraction, and stored at −80° C. The release of human cytokines was measured by ELISA using ELISA MAX Deluxe set (Biolegend) for INFγ and the DuoSet system (DY-2906-05, Bio-Techne) for granzyme B, according to the manufacturer's recommendations. In the TNBC model, the tumour was also explanted, photographed with a millimetric ruler and the volume was calculated as described above.

### Quantification of Metastatic Progression

Upon termination of the study, livers of mice engrafted with the TNBC solid tumour model were explanted and fixed in 10% neutral buffered formalin (CellPath limited) for 24 h at room temperature before processing in paraffin blocs. Paraffin sections were prepared for immunofluorescence, deparaffinized 3×5 min in xylene, rehydrated in 3×5 min washes with 100% ethanol and equilibrated in H2O. Sections were washed 10 min in PBS and a detergent-based antigen retrieval step was performed with a 10 min incubation in PBS+0.05% Tween 20 (PBS.T) and 0.5% Triton X-100. The sections were then washed 3×5 min in PBS.T and non-specific binding sites were saturated with 3% bovine serum albumin (BSA) in PBS.T (w/v) for 1 h at room temperature (RT). The sections were incubated overnight at 4° C. with a goat anti-luciferase (NB1000-1677SS, Novus Biologicals) diluted 1:200 in PBS.T+1% BSA to detect infiltrated MDA-MB-231 cancer cells. The slides were washed 3×5 min in PBS.T and incubated with an Alexa-555 donkey anti-goat (A32816, Thermofisher) polyclonal antibody diluted 1:2000 in PBS.T+1% BSA for 1 h at RT on an orbital shaker. A final 3×5 min wash step was performed before the slides were mounted under coverslip with a drop of ProLong Diamond antifade reagent with DAPI (ThermoFisher Scientific) and allowed to dry for 24 h in the dark. Images were acquired on a Phenolmager HT slide scanner (Akoya Bioscience) and metastatic nodules in each section were manually quantified with their diameter measured using Fiji software (Open-source software, GNU general public license).

### Production of CAR-T Cells

Lentiviruses encoding the ROR1 targeting CAR receptor or the AR_CAR variants were produced by triple transfection of the human embryonic kidney (HEK)-293T cell line, using a second-generation lentiviral packaging system composed of pMD2.G for the viral envelope, pCMV delta R8.2 for the packaging and a transgene plasmid containing the CAR expression cassette. Cells were seeded in a 10 cm dish at 1.65×10⁶ cell per dish in Iscove's Modified Dulbecco's Medium (IMDM, Gibco) supplemented with 10% FBS and incubated overnight. Seeded cells were transfected with GeneJuice (Millipore) as per the manufacturer's instructions. Supernatant containing functional lentiviruses was harvested after 48 h and used immediately or stored at −80° C.

Peripheral blood mononuclear cells were obtained from healthy volunteers and purified as previously described on a density gradient using Ficoll-Paque Plus (GE Healthcare). Isolated PBMCs were activated overnight with human T-Activator CD3/CD28 Dynabeads (ThermoFisher) and 100 U/mL of human IL-2 (Miltenyi). Transduction was performed on RetroNectin coated 24-well plates (Takara). 3×10⁵ activated PBMCs in 0.5 mL of RPMI media supplemented with 10% FBS are dispensed in each well and completed with 1.5 mL of supernatant containing the lentiviruses and 100 U/mL of human IL-2. Transduced cells are incubated for 5 days in the RetroNectin coated plate before evaluation of the transduction efficacy by flow cytometry. Transduced cells centrifugated at 300 g for 5 min and washed once in PBS. Cells are then incubated with PE-labelled protein L (ACROBiosystems) diluted 1:50 in PBS for 60 min at 4° C. to detect expression of the CAR receptor. Stained cells are washed once in PBS and analysed by flow cytometry using a CytoFlex (Beckman Coulter Life Sciences).

Granzyme B Cleavage with CAR-T Cells

Granzyme B mediated cleavage of the AR_CAR receptor was evaluated in vitro. 1×10⁵ CAR-T cells were resuspended in 200 uL of RPMI media and incubated with 100 nM of activated human granzyme B for 2 h at 37° C. The cells were centrifugated 5 min at 300 g and washed once in PBS. The presence of residual CAR receptor at the surface of the cells was detected by flow cytometry using the protein L detection method as described previously.

Short Term Coculture Assay with CAR-T Cells

AR_CAR-T cells were tested in a similar short-term coculture assay as described previously for TCEs. A fixed concentration of ROR1+ Jeko-1 cells was mixed with different amounts of CAR-T cells to achieved a range of effector to target ratio between 0.04:1 (excess of target cells) to 10:1 (excess of CAR-T cells), and cocultured for 48 h. Cytotoxicity was measured as the percentage of target cell death discriminated by positive staining for the Live/Dead marker, while CAR-T cells activation was measured by the Mean Fluorescence Intensity (MFI) of cell surface CD69 staining in flow cytometry.

Long-Term Coculture Assay with CAR-T Cells

Serial engagement and killing of CAR-T cells was tested in the long-term coculture assay with an excess of target cells. CAR-T cells were mixed with an excess of Jeko-1 cells at a 1:10 effector to target ratio and cocultured for up to 5 days. Coculture samples were harvested every 24 h target cell death was measured as described previously by flow cytometry.

### Example 3—Design of a Prototype FVIII Mimetic Bispecific Antibody with Embedded Autoregulation Peptides

Prothrombin (coagulation factor II) is a central element in the clotting cascade and its activation into thrombin (FIIa) is one of the major output of the coagulation process. Abnormally elevated levels of this enzyme are also the hallmark of thrombosis and prothrombotic events. Thus, using the elevation of thrombin enzymatic activity as a trigger to autoregulate bispecific antibodies with FVIII mimetic activity (such as emicizumab) has strong potential to prevent the occurrence of thrombotic events.

To design AR_Ab8, a prototype of FVIII mimetic bispecific antibody with autoregulation capability controlled by thrombin levels, the P4 to P4′ 8 amino-acid long peptide (LTPRGVRL) identified as the consensus recognition sequence for thrombin-mediated cleavage by Gallwitz and collaborators (Gallwitz, Enoksson et al. 2012) was inserted in the sequence of emicizumab. To enable degradation and inactivation of the autoregulated bispecific antibody the peptide was inserted within the hinge region of both heavy chain between the residues K₂₂₈ and Y₂₂₉ (FIG. 2). Thrombin-mediated cleavage will thus yield 3 non-functional fragments comprising the Fc region and the 2 separated Fab domains.

Following production and purification, the migration pattern of AR_Ab8 was compared to that of emicizumab by electrophoresis in non-reducing condition. Both constructs displayed a similar profile with the strong predominance of intact antibodies (FIG. 3A). In particular, degradation products could not be observed with AR_Ab8 indicating the absence of premature non-specific cleavage of the thrombin-sensitive peptide during the production and purification process.

### Example 4—In Vitro Procoagulant Activity of a Prototype FVIII Mimetic Antibody with Thrombin-Mediated Autoregulation

To evaluate whether structural modifications of the hinge region resulting from the insertion of the thrombin-sensitive peptide had a disruptive effect on therapeutic function, the FVIII mimetic potential of AR_Ab8 and emicizumab was evaluated in vitro.

Both antibodies successfully catalysed the FIXa-mediated conversion of FX into FXa in a chromogenic assay with overlapping kinetic curves (FIG. 3B) suggesting that insertion of the cleavable peptide in AR_Ab8 does not impact its ability to bridge FIXa and FX to restore FVIII-like activity. In addition, in a human FVIII deficient plasma where clotting time in the aPTT assay is severely increased compared to a pool of healthy volunteer (mean±SD; 95.6±12.6 versus 29.6±0.7 sec respectively), emicizumab and AR_Ab8 spiked at 350 nM correct the clotting time (22.0±0.3 and 28.1±2.0 respectively) to values that are comparable with the normal control and with no significant difference between both antibodies (FIG. 3C).

### Example 5—Uncompromised Therapeutic Potential of Autoregulated FVIII Mimetic Antibody In Vivo

The procoagulant potential of AR_Ab8 and its parental antibody emicizumab was then tested in vivo in a bleeding assay using the haemophilia A mice model (FVIIIKO) and specifically optimised for FVIII mimetic bispecific antibodies as described by Ferriere and collaborators (Ferriere, Peyron et al. 2020). In this model, were the 3 mm distal tip of the tail is severed and bleeding is monitored over a period of 30 min, non-treated FVIIIKO animals display a strong bleeding phenotype with an average bleeding volume of 900±92 μL (mean±SD). Control animals treated with 2 U/mice of recombinant human FVIII show full correction of the bleeding tendency (FIG. 4A). As expected, when the mice are pre-treated 24 h before the study with a single I.V. injection of 3 mg/kg of emicizumab, partial correction of bleeding can be observed with an average blood loss reduced to 677±198 μL. Interestingly, the treatment with an equivalent dose of AR_Ab8 corrects the bleeding phenotype in the same proportion as the parental antibody emicizumab with an average blood loss of 609±182 μL thus demonstrating that insertion of the thrombin-sensitive peptide in the core structure of the bispecific antibody does not compromise its procoagulant potential and therapeutic efficacy (FIG. 4B).

### Example 6—Thrombin-Mediated Inactivation of Autoregulated FVIII Mimetic Antibody

The insertion of a thrombin-cleavable peptide in the structure of AR_Ab8 enables this prototype autoregulated antibody to be inactivated and degraded when exposed to elevated concentration of thrombin. The sensitivity of AR_Ab8 to thrombin-mediated cleavage was therefore evaluated in vitro.

When exposed to 2 U/mL of human α-thrombin for up to 180 min, the parental antibody emicizumab (at 2 nM) showed no signs of cleavage with only the band corresponding to intact antibodies being detectable by western blot (FIG. 5A). In contrast, AR_Ab8 was progressively cleaved with degradation fragments detectable as soon as 15 min of exposure. In addition, the presence of 2 sets of fragments detectable at approximately 90-100 kDa and 50-55 kDa indicates that both autoregulation peptides present in AR_Ab8 are efficiently cleaved, with almost complete degradation of the autoregulated antibody after 180 min of exposure (FIG. 5A). These results were confirmed using the chromogenic assay to evaluate the residual FVIII mimetic activity of each samples following exposure to α-thrombin. As expected, while the activity of emicizumab remained unchanged, AR_Ab8 showed a significantly reduced FVIII mimetic activity as soon as 30 min of exposure to α-thrombin with a progressive diminution of residual activity. After 180 min of exposure, almost complete inactivation was achieved with an average residual activity reduced to only 1.5±1.3% of that of emicizumab (FIG. 5B). The sensitivity to thrombin exposure of several cleavable peptides was then compared. A FVIII-mimetic antibody construct was designed as a bispecific tandem-scFv and contained (or not) different thrombin-sensitive peptides. The peptides tested were the consensus LTPRGVRL previously described as well as peptides with a single amino-acid modification in the P1 position (LTPRDVRL and LTPRLVRL) and the naturally occurring cleavage site of human coagulation Factor V (FV) in position R1753 (WYLRSNNG). When exposed to α-thrombin for 20 min, the peptides showed different levels of sensitivity as showed by the residual level of FVIII mimetic activity (FIG. 5C). Indeed the consensus (LTPRGVRL) cleavage site displayed the highest sensitivity with only 17.5% of residual activity while the LVRL site showed much lower sensitivity to thrombin exposure with 56.7% of residual FVIII mimetic activity and the DVRL site showed no cleavage at all. Interestingly, the naturally occurring FV_R1573 cleavage site only showed marginal inactivation when exposed to thrombin with 88.7% of retained FVIII mimetic activity after 20 min of exposure (FIG. 5C).

To determine whether thrombin-mediated inactivation could effectively reduce the thrombotic risks of FVIII mimetic antibodies, the thrombin generation profile of both AR_Ab8 and emicizumab was tested in combination with activated Prothrombin Complex Concentrate (aPCC). This assay, previously described by Hartmann and collaborators (Hartmann, Feenstra et al. 2018), mimic the prothrombotic potential of emicizumab observed in the clinic when co-administered with this bypassing agent. Indeed, the combination of 600 nM of emicizumab and 0.5 U/mL of aPCC in haemophilia A plasma yields an abnormally high amount of thrombin generation with a maximum peak increased by 1.79 fold when compared to a pool of normal plasma (FIG. 6A-B). However, thrombin generation was significantly lower in presence of AR_Ab8, with a peak value not significantly different from that of the normal plasma (1.15 fold) suggesting that autoregulation was efficiently triggered to prevent excessive thrombin generation (FIG. 6B).

### Example 7—Thrombin-Mediated Autoregulation Reduces the Pro-Thrombotic Phenotype Associated with FVIII Mimetic Antibody In Vivo

The potential of thrombin-mediated autoregulation to control the therapeutic activity of FVIII mimetic bispecific antibodies and reduce thrombotic risks was then evaluated in vivo using a mice model that recapitulate the prothrombotic potential of emicizumab associated with aPCC. C57Bl/6 mice received an initial injection with 2.5 U of aPCC either alone or in combination with 250 ug of emicizumab or AR_Ab8. All animals then received 3 additional administration of aPCC every 24 h to sustain elevated levels of the bypassing agent and potentialize the prothrombotic phenotype (FIG. 7A).

The total platelet count was measured in blood samples taken from the mice 30 minutes after the last aPCC injection and, as expected, the combination of emicizumab and aPCC induced a significant platelet consumptive coagulopathy with an average platelet count reduced to 621×10³ platelets per μL, where the control group with aPCC alone was 908×10³ platelets per μL (FIG. 7B). However, in mice treated with the combination of aPCC and AR_Ab8, platelet counts in the peripheral blood were completely corrected with a mean 958×10³ platelets per μL which was not significantly different from the control group, thus indicating that the presence of thrombin-mediated autoregulation efficiently managed the risks of coagulopathy associated with FVIII mimetic antibodies (FIG. 7B).

The formation of thrombus in the lungs was then evaluated using an immunofluorescent microscopy approach (FIG. 8). Immunofluorescent staining for PECAM (blood vessels) and integrin αIIb (platelets) were then performed and further confirmed these structures to be platelet-rich thrombi partially occluding the pulmonary blood vessels (FIG. 8A). These images of immunofluorescent staining were subsequently used to define a quantification mask and measure the percentage of blood vessel occlusion defined as the percentage of positive platelet staining within the delimited area of the vessels (FIG. 8A). The blinded quantification of lung sections showed a visible increase in the average rate of occlusion in mice treated with emicizumab when compared to the control group that only received aPCC with the presence of several blood vessel with a percentage of lumina occlusion above 20% (FIG. 8B). In contrast, mice treated with the autoregulated antibody AR_Ab8 displayed a reduced occurrence of highly occluded blood vessels, with a distribution of occlusion rate closer to that of the control group (FIG. 8B), again indicating the ability of thrombin-mediated autoregulation to reduced the risks of adverse events associated with FVIII mimetic antibodies.

### Example 8—Design of ROR1×CD3 Bispecific Engagers with Granzyme B Sensitive Autoregulation Peptides

To demonstrate that autoregulation can be achieved using non-canonical antibody format, in a different indication, bispecific T cell engagers (TCE) with embedded autoregulation were designed to prevent the occurrence of adverse events related to their mode-of-action (MoA), one of the major risk associated with this class of therapeutic agent. Granzyme B is an enzyme specific of immune cells and, in the context of T cell redirection immunotherapies such as TCEs, is released by activating T cells. Interestingly, circulating levels of granzyme B are strongly increased in patients progressing toward severe MoA-related adverse events and this enzyme is therefore and interesting target to trigger the autoregulation of TCE and reduce the risk of such MoA-related toxicities associated with T cell redirection immunotherapies.

The autoregulated bispecific T cell engagers (AR_TCEs) were designed based on NVG-111, a ROR1×CD3 bispecific tandem_scFv (td_scFv). The central GSGGGGS linker was replaced by granzyme B cleavable peptides (8 amino-acid long) flanked on each side with a GGGGS extensor (FIG. 9A). Several granzyme B sensitive peptides were initially tested, AR_TCE-1 and AR_TCE-2 were designed with the peptides GZMB-1 and GZMB-2 respectively derived from the mouse or human P4-P4′ granzyme B cleavage sequence from BH3 interacting-domain death agonist (BID), a well described target of granzyme B. AR_TCE-3 received the peptide GZMB-3 derived from a granzyme B cleavage consensus sequence described by Wee and collaborators (Wee, Er et al. 2011) (FIG. 9B). All constructs were expressed and purified from mammalian cells before the migration pattern was compared to that of the parental antibody NVG-111 by electrophoresis in reducing conditions. All constructs displayed a discrete band at approximately 55 kDa, which is the expected size of td_scFv antibodies, indicting the absence of premature cleavage of the granzyme B sensitive peptides and degradation of the autoregulated constructs (FIG. 9C).

### Example 9—Cytotoxicity and T Cell Engagement Potential of Autoregulated AR_TCEs

The cytotoxic and T cell engagement capacity of AR_TCEs was evaluated side-by-side with the parental NVG-111 in a short term killing assay. ROR1+ Jeko-1 target cells were co-incubated with an excess of purified human T cells using a 5:1 Target to Effector ratio and in presence of increasing concentrations of either antibodies for 48 h. Both AR_TCE-1 and AR_TCE-3 showed similar T cell activation potential when compared to the parental NVG-111, as measured by cell surface expression of CD69 (FIG. 10A). In addition, all 3 constructs induced comparable T cell mediated target cell killing (FIG. 10B), thus demonstrating that insertion of granzyme B cleavable peptides in the linker of autoregulated AR_TCEs does not compromise their mode of action in vitro.

### Example 10—Autoregulation Enables Threshold-Dependent Inactivation of TCE by Exposure to Granzyme B

To determine whether the presence of granzyme B cleavable peptides inserted in the AR_TCEs does provide efficient sensitivity to granzyme B exposure, several AR_TCE variants were tested in an in vitro cleavage assay. When incubated with 100 nM of recombinant activated granzyme B for 2 h, both AR_TCE-1 and AR_TCE-2 showed partial cleavage with a faint band between 25 and 30 kDa detectable by electrophoresis and protein L immunoblotting, corresponding to the cleaved single scFvs. Residual intact material was also present around 55 kDa (FIG. 11A). In contrast, the AR_TCE-3 variant displayed a much higher sensitivity to granzyme B with complete cleavage achieved in 2 h and no detectable intact material (FIG. 11A).

The subsequent detection by ELISA of residual intact antibodies showed, as expected, a complete absence of effect on the parental antibody NVG-111 when exposed to increasing concentration of granzyme B (FIG. 11B). In contrast, AR_TCE-1 showed a minor reduction in the level of intact antibodies at the highest concentration of 100 nM coherent with the partial degradation observed by Western Blot, while AR_TCE-3 was progressively cleaved with increasing concentrations of granzyme B until complete inactivation (FIG. 11B). This observation highlights the dose-dependent and threshold-mediated inactivation mechanism of autoregulation in TCEs where AR_TCE-3 remains intact at low levels of granzyme B while showing complete inactivation at high concentrations associated with higher risks of severe MoA-related adverse events.

### Example 11—Specificity and Sensitivity of Granzyme B Cleavable Peptides Used in Autoregulation

To further explore the relationship between autoregulation peptide sequence and their sensitivity to granzyme B exposure, several additional peptides were generated from the consensus peptide GZMB-3 by modulating the P4-P4′ sequence. These new peptides (GZMB-4 to -14) were further used to generate additional AR_TCE variants as previously described (FIG. 12A). Interestingly, when exposed to a single dose of granzyme B (50 nM) the diversity of autoregulation peptides demonstrated a broad range of cleavage sensitivity from constructs similar to the parental antibody NVG-111 showing little to no signs of inactivation (AR_TCE-2, -13) and up to strongly sensitive variants (AR_TCE-3, -8) on the other end of the spectrum with only about 25% of intact antibody remaining after 2 h of exposure (FIG. 12A). This diversity in granzyme B sensitivity supports the ability to fine-tune the kinetic of autoregulation and select a specific sequence that will provide inactivation of AR_TCEs only at level of granzyme B that are associated with MoA-related adverse events.

In addition, the specificity of the GZMB-3 peptide as a substrate for the proteolytic activity of granzyme B was also evaluated in vitro. AR_TCE-3 was exposed for 2 h to an equimolar concentration (50 nM) of a panel of proteases selected for their reported presence either in circulation or within the tumour microenvironment, and representative of the different protease families. In this assay, AR_TCE-3 shows a unique proteolytic sensitivity to granzyme B with significant inactivation of the molecule, while remaining intact when exposed to any other proteases tested in the panel (FIG. 12B). Such selectivity toward the protease used to trigger autoregulation reduces significantly the risks of non-specific, premature inactivation of the AR_TCEs.

### Example 12—Granzyme B Mediated Autoregulation Reduces Cytokine Release Induced by TCE In Vitro

Two AR_TCEs with low (AR_TCE-1) and high (AR_TCE-3) sensitivity to granzyme B exposure were characterised alongside NVG-111 in a long-term killing assay which evaluates serial target cell killing and progressive T cell engagement. Purified human T cells were cocultured with an excess of ROR+ Jeko-1 target cells (1:10 effector to target ratio) and in presence of the bispecific antibodies. Similarly to the previous observation in short-term killing assay (FIG. 10) the cytotoxic potential of all 3 antibodies was identical over the 120 h of coculture with a maximum target cell death of 60.5, 58.7 and 55.7% for NVG-111, AR_TCE-1 and AR_TCE-3 respectively (FIG. 13A). However, long term T cell activation measured by the increasing cell surface expression of CD25 (IL-2 receptor a subunit), started to progressively plateau from 96 h onward with the AR_TCEs when compared to NVG-111, with the strongest effect being observed for the high sensitivity AR_TCE-3 molecule. Indeed, at 120 h the mean CD25 expression was reduced 14.2 and 21.5% in AR_TCE-1 and AR_TCE-3 respectively (FIG. 13B).

In addition, the apparent reduction in T cell activation was correlated with a strong and significant reduction in the release of human Interferon γ (INFγ). At 120 h, T cells cocultured in presence of AR_TCE-1 and AR_TCE-3 released levels of INFγ respectively equivalent to 77.3 and 52.3% that of cells cocultured with the parental NVG-111 (FIG. 13C). This reduction is cytokine release was also correlated with a visible autoregulation and inactivation of the AR_TCEs. Indeed, while NVG-111 remains intact in the coculture supernatant for the entire duration of the assay, AR_TCE-1 and AR_TCE-3 showed evidence of cleavage with the presence of fragmentation by-products (FIG. 13D). As expected, antibody fragments were only detected at 120 h for the low sensitivity AR_TCE-1 construct while inactivation was observed as soon as 72 h for the more sensitive AR_TCE-3 molecule (FIG. 13D). Altogether these results demonstrate that antibody engineering to incorporate a granzyme B mediated autoregulation mechanism in a TCE can significantly reduce cytokine release induced by T cells engagement and activation, while preserving unaffected cytotoxic potential. In addition, the kinetic of antibody inactivation and therefore the impact on cytokine release can be modified through careful selection of the autoregulation peptide sequence.

### Example 13—Autoregulation Reduces the Onset of In Vivo Severe Toxicities Related to TCE Mode-of-Action, Such as Cytokine Release Syndrome, in a Model of Triple Negative Breast Cancer

The mechanism of granzyme B mediated autoregulation was further challenged in an in vivo model reproducing the features of MoA-related severe toxicities associated with TCEs. In this model, immunocompromised NSG mice are induced to expressed stable levels of either NVG-111 or the AR_TCE-3 variants, at a concentration (average ranging from 0.66 to 2.01 μg/mL) which is known to produce rapid toxicity with the non-regulated parental antibody (FIG. 14A). The mice are then engrafted subcutaneously with the ROR1+ MDA-MB-231 triple negative breast cancer (TNBC) cell line and the resulting solid tumour is treated with 6 cycles of purified human T cells for a period of 21 days (from day 25 to day 46).

Weight loss and animal survival are the primary outcomes of TCE induced toxicity in this model. As expected, animals treated with NVG-111 showed rapid signs of toxicity 24 h after the second T cell injection with a sudden and significant loss of total body weight as soon as day 30 (FIG. 14B). The subsequent aggravation of weight loss and global deterioration of animal wellbeing led to the ethical culling of 4 out of 6 mice by day 36 (FIG. 14C) and the remaining animals were removed from the protocol. In contrast, animals treated with the autoregulated AR_TCE-3 molecule showed no signs of toxicity with a stable weight fluctuation identical to that of the vehicle group (FIG. 14B). As a consequence, all animal in this group were maintained in protocol until the ends of the study at day 46 (FIG. 14C). Interestingly, the results suggest that the response of autoregulation to granzyme B release is extremely rapid and can control the onset of toxicity arising within 24 h of the T cell injection. Human cytokines released from T cells were also measured in plasma samples collected at the day of culling for both groups of mice. Animals treated with AR_TCE-3 had significantly reduced circulating levels of both human IFNγ and granzyme B when compared to NVG-111, thus indicating a reduced T cell activation correlating with improved survival (FIG. 15).

### Example 14—Autoregulated AR_TCE Showed Uncompromised Anti-Tumour Efficacy In Vivo

To evaluate whether the reduced toxicities and cytokine release observed in mice treated with the autoregulated TCE was associated to a reduction in therapeutic efficacy, tumour progression was measured in each group. AR_TCE-3 showed apparent uncompromised efficacy with a significantly reduced tumour progression detectable as early as day 34 when compared with the vehicle group (FIG. 16A). Indeed, upon completion of the study, tumour volume had progressed by 18.5±0.94 fold from day 15 (mean±SD) in the vehicle group but only 8.0±3.4 fold in the AR_TCE-3 treated animals. In addition, in 3 out of 6 mice in the vehicle group, the tumour progressed to an ulcerated stage which trigger an early ethical culling at day 40 (FIG. 16A). These data were supported by the measure of tumour explant volumes post-mortem (FIG. 16B) with an average 910 mm³ in the vehicle group and only 578 mm³ in animals treated with AR_TCE-3 (FIG. 16C). Interestingly, while the data are incomplete due to the early culling of NVG-111 treated mice, the tumour progression is this group show no difference up to day 36 when compared to mice that had received AR_TCE-3 (progression of 4.6±2.3 and 5.2±1.7 fold respectively) which support the uncompromised efficacy of autoregulated AR_TCEs (FIG. 16A).

### Example 15—Autoregulation Enables Sustained Treatment with AR_TCE and Reduces TNBC Metastatic Proliferation In Vivo

The improved safety profile AR_TCE-3 enabled a longer treatment phase in mice when compared to the parental NVG-111 (FIG. 14). Therefore, the long-term therapeutic efficacy of sustained exposure to AR_TCE-3 was evaluated on the metastatic progression of the TNBC model. While animals in the vehicle group presented apparent metastatic nodules at the surface of the liver (FIG. 17A), which were confirmed by the presence of cancer cell clusters in liver sections (FIG. 17B), such nodules were not visible in mice treated with AR_TCE-3. Quantification of metastatic nodules detected by immunofluorescent staining further showed a significant reduction in the number of large cancer cell clusters (d>100 um) infiltrating the liver tissues in animals treated with AR_TCE-3 when compared with mice in the vehicle group (FIG. 17C) thus confirming that AR technology does not impact therapeutic efficacy while the reduced toxicities enables extension of the therapeutic window compared to NVG-111.

Overall, the results obtained in this model of severe toxicities associated with the mode-of-action of TCEs demonstrated that the built-in of autoregulation through granzyme B cleavable peptides can efficiently control the risk of severe adverse events inherent to T cell engager therapies, and therefore enables longer treatments with sustained therapeutic efficacy against tumour progression and metastatic dissemination.

### Example 16—Autoregulation Improves Survival In Vivo while Preserving Therapeutic Efficacy in a Model of Pancreatic Cancer

The results obtained in the TNBC model were further confirmed with a different cancer type using a shorter xenograft model established by intraperitoneal injection of the ROR+ PANC-1 pancreatic cancer cell line and 14 days of treatment phase with 4 cycles of human T cells administration (FIG. 18A). Similarly to the outcome of the TNBC xenograft model, NVG-111 treated mice showed rapid signs of toxicity following T cell administration, with an elevated mortality rate as ethical culling was required for 4 out of 6 mice between day 16 and 20. In contrast only 1 out of 6 animals treated with autoregulated AR_TCE-3 required ethical culling at day 21 (FIG. 18B).

To evaluate cytokine release in response to T cells engagement, a single blood draw was performed in all groups of mice 24 h after the second injection (e.g. day 13). AR_TCE-3 treated mice showed significantly lower levels of granzyme B when compared to NVG-111 and an apparent lower release of IFNγ suggesting that in a 24 h time frame, the autoregulation mechanism of AR_TCE-3 had efficiently controlled T cell activation (FIG. 18C). Therapeutic efficacy of the AR_TCE was further validated through the bioluminescent reading of tumour progression. In 3 out of 5 animals in the vehicle group, multiple foci of tumour proliferation could be observed upon termination of the study (day 22) while all mice treated with AR_TCE-3 were cleared of tumour progression (FIG. 19).

### Example 17—Application of Granzyme B Mediated Autoregulation to a Model of ROR1 Targeting CAR-T Cells

To demonstrate the applicability of autoregulation to genetically engineered cells in the context of T cell redirecting therapies, autoregulated AR_CAR-T cell variants were designed from a ROR1 targeting CAR-T cell therapy. The 8 residues (sequence P4 to P4′) of the granzyme B sensitive peptide GZMB-3, previously used in autoregulated AR_TCE-3, was inserted in the upper region of the chimeric receptor (FIG. 20A). The autoregulation peptide was either directly inserted in-between the ROR1 targeting scFv and the hinge region of the chimeric receptor (AR_CAR-T27) or by substitution of the first 5 residues of the hinge region (AR_CAR-T22).

Both autoregulated AR_CAR-T variants as well as the parental CAR-T cells were produced by lentiviral infection of PBMC obtained from healthy donors. The CAR-T cells were then exposed to 100 nM of activated human granzyme B for 2 h in vitro to evaluate the sensitivity of AR_CAR-T variants to the proteolytic activity of granzyme B. When assessed by flow cytometry, both AR_CAR-T22 and AR_CAR-T27 showed significant cleavage and removal of the chimeric receptor from the cell surface, with 39.5 and 38.0% of residual CAR receptor detected by protein L (FIG. 20B). In contrast, the parental CAR-T cell was unaffected by exposure to granzyme B.

### Example 18—Autoregulation Maintains the Cytotoxic Potential and Serial Engagement Capability of AR_CAR-T Cells

The cytotoxicity of AR_CAR-T cells was compared to the parental ROR1 targeting CAR-T cell in a short-term killing assay with various cell concentrations. The ROR1+ Jeko-1 target cell line was co-incubated for 48 h with CAR-T cells using effector to target ratios ranging from 0.04 (excess of target cells) to 10 (excess of CAR-T cells). Interestingly, both autoregulated AR_CAR-T cell variants showed similar activation levels the parental CAR-T cell for all tested cell ratios (FIG. 21A). In addition, activation levels were correlated with comparable target cell killing (FIG. 21B). The function and ability of autoregulated AR_CAR-T cells to promote serial killing was then evaluated in a long-term killing assays which reflect progressive CAR-T cell engagement. CAR-T cells were cocultured with an excess of ROR+ Jeko-1 target cells (1:10 effector to target ratio) for up to 5 days. Similarly to the previous observations, CAR-T cell engagement and cytotoxicity was uncompromised in presence of autoregulation. Progressive killing was observed with a maximal target cell death observed at 120 h with 72.4, 74.8 and 79.1% for AR_CAR-T22, AR_CAR-T27 and the parental CAR-T cell respectively (FIG. 22)

These results indicate that the modifications of the CAR receptor to introduce the granzyme B sensitive peptide do not compromise the CAR-T cell function and therefore demonstrate that autoregulation can be successfully applied to genetically engineered CAR-T cells to enable granzyme B mediated inactivation and mitigate the risk of severe adverse events associated with the mode of action of CAR-T cells.

Sequence listing

SEQ ID NO: 1-Thrombin cleavage recognition sequence consensus sequence
LTPRGVRL
SEQ ID NO: 2-Thrombin cleavage recognition sequence P1′ G to L
LTPRLVRL
SEQ ID NO: 3-Thrombin cleavage recognition sequence P1′ G to D
LTPRDVRL
SEQ ID NO: 4-Thrombin cleavage recognition sequence from human coagulation Factor V (FV)
WYLRSNNG
SEQ ID NO: 5: Granzyme B cleavage recognition sequence GZMB-1, from mouse BID protein
IEPDSESQ
SEQ ID NO: 6: Granzyme B cleavage recognition sequence GZMB-2, from mouse BID protein
IEADSESQ
SEQ ID NO: 7: Granzyme B cleavage recognition sequence GZMB-3, consensus
IEPDSLEE
SEQ ID NO: 8: Granzyme B cleavage recognition sequence GZMB-4, P10-P10′ sequence of GZMB-2
HSRLGRIEADSESQEDIIRN
SEQ ID NO: 9: Granzyme B cleavage recognition sequence GZMB-5, P4 I to V
VEPDSLEE
SEQ ID NO: 10: Granzyme B cleavage recognition sequence GZMB-6, P4 I to L
LEPDSLEE
SEQ ID NO: 11: Granzyme B cleavage recognition sequence GZMB-7, P2 P to G
IEGDSLEE
SEQ ID NO: 12: Granzyme B cleavage recognition sequence GZMB-8, P2 P to A
IEADSLEE
SEQ ID NO: 13: Granzyme B cleavage recognition sequence GZMB-9, P1′ S to A
IEPDALEE
SEQ ID NO: 14: Granzyme B cleavage recognition sequence GZMB-10, P2′ L to E and P4′ E to Q
IEPDSEEQ
SEQ ID NO: 15: Granzyme B cleavage recognition sequence GZMB-11, P2′ L to E and P4′ E to V
IEPDSEVE
SEQ ID NO: 16: Granzyme B cleavage recognition sequence GZMB-12, P1 D to N
IEPNSLEE
SEQ ID NO: 17: Granzyme B cleavage recognition sequence GZMB-13, P1 D to E
IEPESLEE
SEQ ID NO: 18: Granzyme B cleavage recognition sequence GZMB-14, P1 D to Q
IEPQSLEE
SEQ ID NO: 19: ROR1-binding antigen binding domain LCDR 1
QNIDRY
SEQ ID NO: 20: ROR1-binding antigen binding domain LCDR 2
NTN
SEQ ID NO: 21: ROR1-binding antigen binding domain LCDR 3
LOYNSLPLT
SEQ ID NO: 22: ROR1-binding antigen binding domain HCDR 1
GFIFSEHN
SEQ ID NO: 23: ROR1-binding antigen binding domain HCDR 2
ISDDGRNT
SEQ ID NO: 24: ROR1-binding antigen binding domain consensus HCDR 3
XXHRYNLFDS (where X1 is A or T and X2 is S, K or R)
SEQ ID NO: 25: ROR1-binding antigen binding domain HCDR 3, Clone F Heavy Chain CDR3
ASHRYNLFDS
SEQ ID NO: 26: ROR1-binding antigen binding domain HCDR 3, Humanised 1 Heavy Chain CDR3
TSHRYNLFDS
SEQ ID NO: 27: ROR1-binding antigen binding domain HCDR 3, Humanised 2 and 5 Heavy Chain CDR3
AKHRYNLFDS
SEQ ID NO: 28: ROR1-binding antigen binding domain HCDR 3, Humanised 3 and 4 Heavy Chain CDR3
ARHRYNLFDS
SEQ ID NO: 29: ROR1-binding antigen binding domain, Clone F light chain variable region (CDRs
underlined)
DIQMTQSPSFLSASVGDRVTINCKASQNIDRYLNWYQQKLGEAPKRLLYNTNKLQTGIPSRFSGSGSATDFTL
TISSLQPEDFATYFCLQYNSLPLTFGSGTKLEIK
SEQ ID NO: 30: ROR1-binding antigen binding domain, Humanised 1 light chain variable region (CDRs
underlined)
DIQMTQSPSSLSASVGDRVTITCKASQNIDRYLNWYQQKPGKAPKRLIYNTNKLQTGVPSRFSGSGSGTEFTL
TISSLQPEDFATYYCLQYNSLPLTFGQGTKLEIK
SEQ ID NO: 31: ROR1-binding antigen binding domain, Humanised 2 light chain variable region (CDRs
underlined)
DIQMTQSPSSLSASVGDRVTITCKASQNIDRYLNWFQQKPGKAPKSLIYNTNKLQTGVPSKFSGSGSGTDFTL
TISSLQPEDFATYYCLQYNSLPLTFGQGTRLEIK
SEQ ID NO: 32: ROR1-binding antigen binding domain, Humanised 3 light chain variable region (CDRs
underlined)
DIQMTQSPSSLSASVGDRVTITCKASQNIDRYLNWYQQKPGKAPKLLIYNTNKLQTGVPSRFSGSGSGTDFTL
TISSLQPEDFATYYCLQYNSLPLTFGQGTKLEIK
SEQ ID NO: 33: ROR1-binding antigen binding domain, Humanised 4 light chain variable region (CDRs
underlined)
DIQLTQSPSFLSASVGDRVTITCKASQNIDRYLNWYQQKPGKAPKLLIYNTNKLQTGVPSRFSGSGSGTEFTL
TISSLQPEDFATYYCLQYNSLPLTFGQGTKLEIK
SEQ ID NO: 34: ROR1-binding antigen binding domain, Humanised 5 light chain variable region (CDRs
underlined)
DIQMTQSPSTLSASVGDRVTITCKASQNIDRYLNWYQQKPGKAPKLLIYNTNKLQTGVPSRFSGSGSGTEFTL
TISSLQPDDFATYYCLQYNSLPLTFGQGTKLEIK
SEQ ID NO: 35: ROR1-binding antigen binding domain, Clone F heavy chain variable region (CDRs
underlined)
EVQLVESGGGLVQPGRSLKLSCAASGFIFSEHNMAWVRQAPKKGLEWVATISDDGRNTYYRDSMRGRFTISRE
NARSTLYLQLDSLRSEDTATYYCASHRYNLFDSWGQGVMVTVSS
SEQ ID NO: 36: ROR1-binding antigen binding domain, Humanized 1 heavy chain variable region (CDRs
underlined)
QVQLVESGGGVVQPGRSLRLSCAASGFIFSEHNMAWVRQAPGKGLEWVATISDDGRNTYYRDSMRGRFTISRD
NSKNTLYLQMNSLRAEDTAVYYCTSHRYNLFDSWGQGTMVTVSS
SEQ ID NO: 37: ROR1-binding antigen binding domain, Humanized 2 heavy chain variable region (CDRs
underlined)
EVQLVESGGGLVQPGGSLRLSCAASGFIFSEHNMAWVRQAPGKGLEWVSTISDDGRNTYYRDSMRGRFTISRD
NSKNTLYLQMNSLRAEDTAVYYCAKHRYNLFDSWGQGTLVTVSS
SEQ ID NO: 38: ROR1-binding antigen binding domain, Humanized 3 heavy chain variable region (CDRs
underlined)
EVQLVESGGGLVQPGGSLRLSCAASGFIFSEHNMAWVRQAPGKGLEWVATISDDGRNTYYRDSMRGRFTISRD
NAKNSLYLQMNSLRAEDTAVYYCARHRYNLFDSWGQGTMVTVSS
SEQ ID NO: 39: ROR1-binding antigen binding domain, Humanized 4 heavy chain variable region (CDRs
underlined)
EVQLVESGGGLVQPGGSLRLSCAASGFIFSEHNMAWVRQAPGKGLVWVSTISDDGRNTYYRDSMRGRFTISRD
NAKNTLYLQMNSLRAEDTAVYYCARHRYNLFDSWGQGTLVTVSS
SEQ ID NO: 40: ROR1-binding antigen binding domain, Humanized 5 heavy chain variable region (CDRs
underlined)
EVQLVESGGGLVQPGRSLRLSCAASGFIFSEHNMAWVRQAPGKGLEWVSTISDDGRNTYYRDSMRGRFTISRD
NAKNSLYLQMNSLRAEDTALYYCAKHRYNLFDSWGQGTLVTVSS
SEQ ID NO: 41: CD3-binding antigen binding domain, mouse light chain variable region (CDRs
underlined)
QIVLTQSPAIMSASPGEKVTMTCSASSSVSYMNWYQQKSGTSPKRWIYDTSKLASGVPAHFRGSGSGTSYSLT
ISGMEAEDAATYYCQQWSSNPFTFGSGTKLEIN
SEQ ID NO: 42: CD3-binding antigen binding domain, Humanised 1 light chain variable region (CDRs
underlined)
EIVLTQSPATLSLSPGERATLSCSASSSVSYMNWYQQKPGQAPRLLIYDTSKLASGIPARFSGSGSGTDFTLT
ISSLEPEDFAVYYCQQWSSNPFTFGQGTKLEIK
SEQ ID NO: 43: CD3-binding antigen binding domain, Humanised 2 light chain variable region (CDRs
underlined)
DIQMTQSPSSLSASVGDRVTITCSASSSVSYMNWYQQKPGKAPKLLIYDTSKLASGVPSRFSGSGSGTDFTLT
ISSLQPEDFATYYCQQWSSNPFTFGQGTKLEIK
SEQ ID NO: 44: CD3-binding antigen binding domain, Humanised 3 light chain variable region (CDRs
underlined)
DIQLTQSPSFLSASVGDRVTITCSASSSVSYMNWYQQKPGKAPKLLIYDTSKLASGVPSRFSGSGSGTEFTLT
ISSLQPEDFATYYCQQWSSNPFTFGQGTKLEIK
SEQ ID NO: 45: CD3-binding antigen binding domain, Humanised 4 light chain variable region (CDRs
underlined)
DIQMTQSPSTLSASVGDRVTITCSASSSVSYMNWYQQKPGKAPKLLIYDTSKLASGVPSRFSGSGSGTEFTLT
ISSLQPDDFATYYCQQWSSNPFTFGQGTKLEIK
SEQ ID NO: 46: CD3-binding antigen binding domain, Humanised 5 light chain variable region (CDRs
underlined)
DIQMTQSPSSLSASVGDRVTITCSASSSVSYMNWYQQKPGKAPKRLIYDTSKLASGVPSRFSGSGSGTEFTLT
ISSLQPEDFATYYCQQWSSNPFTFGQGTKVEIK
SEQ ID NO: 47: CD3-binding antigen binding domain, mouse heavy chain variable region (CDRs
underlined)
QVQLQQSGAELARPGASVKMSCKASGYTFTRYTMHWVKQRPGQGLEWIGYINPSRGYTNYNQKFKDKATLTTD
KSSSTAYMQLSSLTSEDSAVYYCARYYDDHYCLDYWGQGTTLTVSS
SEQ ID NO: 48: CD3-binding antigen binding domain, Humanised 1 heavy chain variable region (CDRs
underlined)
QVQLVQSGAEVKKPGASVKVSCKASGYTFTRYTMHWVRQAPGQGLEWMGYINPSRGYTNYNQKFKDRVTMTRD
TSTSTVYMELSSLRSEDTAVYYCARYYDDHYCLDYWGQGTLVTVSS
SEQ ID NO: 49: CD3-binding antigen binding domain, Humanised 2 heavy chain variable region (CDRs
underlined)
QVQLVQSGAEVKKPGASVKVSCKASGYTFTRYTMHWVRQAPGQRLEWMGYINPSRGYTNYNQKFKDRVTITRD
TSASTAYMELSSLRSEDTAVYYCARYYDDHYCLDYWGQGTLVTVSS
SEQ ID NO: 50: CD3-binding antigen binding domain, Humanised 3 heavy chain variable region (CDRs
underlined)
QVQLVQSGAEVKKPGSSVKVSCKASGYTFTRYTMHWVRQAPGQGLEWMGYINPSRGYTNYNQKFKDRVTITAD
KSTSTAYMELSSLRSEDTAVYYCARYYDDHYCLDYWGQGTMVTVSS
SEQ ID NO: 51: CD3-binding antigen binding domain, Humanised 4 heavy chain variable region (CDRs
underlined)
QVQLVESGGGLVKPGGSLRLSCAASGYTFTRYTMHWIRQAPGKGLEWVSYINPSRGYTNYNQKFKDRFTISRD
NAKNSLYLQMNSLRAEDTAVYYCARYYDDHYCLDYWGQGTTVTVSS
SEQ ID NO: 52: CD3-binding antigen binding domain, Humanised 5 heavy chain variable region (CDRs
underlined)
EVQLVESGGGLVQPGGSLRLSCAASGYTFTRYTMHWVRQAPGKGLEWVSYINPSRGYTNYNQKFKDRFTISRD
NSKNTLYLQMNSLRAEDTAVYYCAKYYDDHYCLDYWGQGTLVTVSS
SEQ ID NO: 53: CD3-binding antigen binding domain LCDR 1
SASSSVSYMN
SEQ ID NO: 54: CD3-binding antigen binding domain LCDR 2
DTSKLAS
SEQ ID NO: 55: CD3-binding antigen binding domain LCDR 3
QQWSSNPFT
SEQ ID NO: 56: CD3-binding antigen binding domain HCDR 1
GYTFTRYTMH
SEQ ID NO: 57: CD3-binding antigen binding domain HCDR 2
YINPSRGYTNYNQKFKD
SEQ ID NO: 58: CD3-binding antigen binding domain HCDR 3
YYDDHYCLDY
SEQ ID NO: 59: linker sequence
GGGGS
SEQ ID NO: 60: linker sequence
GGGGSGGGGS
SEQ ID NO: 61: ROR1xCD3 bispecific antibody amino acid sequence
QVQLVESGGGVVQPGRSLRLSCAASGFIFSEHNMAWVRQAPGKGLEWVATISDDGRNTYYRDSMRGRFTISRD
NSKNTLYLQMNSLRAEDTAVYYCTSHRYNLFDSWGQGTMVTVSSGGGGSGGGGSGGGGSDIQMTQSPSSLSAS
VGDRVTITCKASQNIDRYLNWYQQKPGKAPKRLIYNTNKLQTGVPSRFSGSGSGTEFTLTISSLQPEDFATYY
CLQYNSLPLTFGQGTKLEIKSGSGGGGSQVQLVQSGAEVKKPGSSVKVSCKASGYTFTRYTMHWVRQAPGQGL
EWMGYINPSRGYTNYNQKFKDRVTITADKSTSTAYMELSSLRSEDTAVYYCARYYDDHYCLDYWGQGTMVTVS
SVEGGSGGSGGSGGSGGVDDIQMTQSPSSLSASVGDRVTITCSASSSVSYMNWYQQKPGKAPKRLIYDTSKLA
SGVPSRFSGSGSGTEFTLTISSLQPEDFATYYCQQWSSNPFTFGQGTKVEIK
SEQ ID NO: 62: ROR1xCD3 bispecific antibody amino acid sequence with an N-terminal hexa-histidine tag
(NVG-111)
QVQLVESGGGVVQPGRSLRLSCAASGFIFSEHNMAWVRQAPGKGLEWVATISDDGRNTYYRDSMRGRFTISRD
NSKNTLYLQMNSLRAEDTAVYYCTSHRYNLFDSWGQGTMVTVSSGGGGSGGGGSGGGGSDIQMTQSPSSLSAS
VGDRVTITCKASQNIDRYLNWYQQKPGKAPKRLIYNTNKLQTGVPSRFSGSGSGTEFTLTISSLQPEDFATYY
CLQYNSLPLTFGQGTKLEIKSGSGGGGSQVQLVQSGAEVKKPGSSVKVSCKASGYTFTRYTMHWVRQAPGQGL
EWMGYINPSRGYTNYNQKFKDRVTITADKSTSTAYMELSSLRSEDTAVYYCARYYDDHYCLDYWGQGTMVTVS
SVEGGSGGSGGSGGSGGVDDIQMTQSPSSLSASVGDRVTITCSASSSVSYMNWYQQKPGKAPKRLIYDTSKLA
SGVPSRFSGSGSGTEFTLTISSLQPEDFATYYCQQWSSNPFTFGQGTKVEIKHHHHHH
SEQ ID NO: 63: common LCDR 1 of FIX-binding and FX-binding antigen binding domains
RNIERQ
SEQ ID NO: 64: common LCDR 2 of FIX-binding and FX-binding antigen binding domains
QAS
SEQ ID NO: 65: common LCDR 3 of FIX-binding and FX-binding antigen binding domains
QQYSDPPLT
SEQ ID NO: 66: FIX-binding antigen binding domain HCDR 1
GFTFSYYD
SEQ ID NO: 67: FIX-binding antigen binding domain HCDR 2
ISPSGQST
SEQ ID NO: 68: FIX-binding antigen binding domain HCDR 3
ARRTGREYGGGWYFDY
SEQ ID NO: 69: FX-binding antigen binding domain HCDR 1
GYTFTDNN
SEQ ID NO: 70: FX-binding antigen binding domain HCDR 2
INTRSGGS
SEQ ID NO: 71: FX-binding antigen binding domain HCDR 3
ARRKSYGYYLDE
SEQ ID NO: 72: common light chain variable region of FIX-binding and FX-binding antigen binding
domains (CDRs in bold)
DIQMTQSPSSLSASVGDRVTITCKASRNIERQLAWYQQKPGQAPELLIYQASRKESGVPDRFSGSRYGTDFTL
TISSLQPEDIATYYCQQYSDPPLTFGGGTKVEIK
SEQ ID NO: 73: heavy chain variable region of FIX-binding antigen binding domain (CDRs in bold)
QVQLVESGGGLVQPGGSLRLSCAASGFTFSYYDIQWVRQAPGKGLEWVSSISPSGQSTYYRREVKGRFTISRD
NSKNTLYLQMNSLRAEDTAVYYCARRTGREYGGGWYFDYWGQGTLVTVSS
SEQ ID NO: 74: heavy chain variable region of FX-binding antigen binding domain (CDRs in bold)
QVQLVQSGSELKKPGASVKVSCKASGYTFTDNNMDWVRQAPGQGLEWMGDINTRSGGSIYNEEFQDRVIMTVD
KSTDTAYMELSSLRSEDTATYHCARRKSYGYYLDEWGEGTLVTVSS
SEQ ID NO: 75: hinge region
ESKYGPPCPPCP
SEQ ID NO: 76: hinge region plus SEQ ID NO: 1 (SEQ ID NO: 1 underlined)
ESKLTPRGVRLYGPPCPPCP
SEQ ID NO: 77: linker sequence
GSGGGGS
SEQ ID NO: 78: linker sequence
GGGGSXXXXXXXXGGGGS
SEQ ID NO: 79: linker sequence
GGGGSIEPDSESQGGGGS
SEQ ID NO: 80: linker sequence
GGGGSIEADSESQGGGGS
SEQ ID NO: 81: linker sequence
GGGGSIEPDSLEEGGGGS
SEQ ID NO: 82: hinge region of CAR embodiment
EPKSPDKTHTCPPCP
SEQ ID NO: 83: partially deleted hinge region of CAR embodiment plus SEQ ID NO: 7
IEPDSLEEDKTHTCPPCP
SEQ ID NO: 84: hinge region of CAR embodiment plus SEQ ID NO: 7
IEPDSLEEEPKSPDKTHTCPPCP

### EMBODIMENTS

1. A therapeutic agent comprising a self-regulation element, wherein the self-regulation element is a small peptide cleavable by an endogenous factor, wherein the activity of the endogenous factor is modified as a consequence of the activity of the therapeutic agent, and wherein cleavage of the self-regulation element results in separation of the therapeutic agent into non-functional fragments.
2. The therapeutic agent of embodiment 1, wherein the endogenous factor is active in the same metabolic pathway as that acted on by the therapeutic agent.
3. The therapeutic agent of embodiment 1 or 2, wherein the endogenous factor is a protease.
4. The therapeutic agent of any one of the preceding embodiments, wherein the self-regulation element comprises a proteolytic cleavage site.
5. The therapeutic agent of any one of the preceding embodiments, wherein the rate of cleavage of the self-regulation element by the endogenous factor is correlated with the level of activity of the therapeutic agent.
6. The therapeutic agent of embodiment 5, wherein the rate of cleavage of the self-regulation element by the endogenous factor is correlated with the level of activity of the therapeutic agent and the peptide sequence of the self-regulation element.
7. The therapeutic agent of any one of embodiments 1 to 6, wherein self-regulation element comprises a thrombin or Granzyme B cleavage recognition site.
8. The therapeutic agent of any one of the preceding embodiments, wherein the therapeutic agent comprises a polypeptide.
9. The therapeutic agent of embodiment 8, wherein the therapeutic agent is an antigen binding protein comprising a first antigen binding domain, wherein the first antigen binding domain comprises a heavy chain variable domain comprising a heavy chain complementarity determining region (HCDR)1, an HCDR2 and an HCDR3, and wherein the self-regulation element is positioned C-terminal to the first antigen binding domain.
10. The antigen binding protein of embodiment 9, wherein the first antigen binding domain is a VHH antibody.
11. The antigen binding protein of embodiment 9, wherein the antigen binding protein comprising a first antigen binding domain, wherein the first antigen binding domain comprises a light chain variable domain and a heavy chain variable domain, wherein the light chain variable domain comprises a light chain complementarity determining region (LCDR)1, an LCDR2 and an LCDR3, and wherein the heavy chain variable domain comprises a heavy chain complementarity determining region (HCDR)1, an HCDR2 and an HCDR3, and wherein the self-regulation element is positioned C-terminal to the first antigen binding domain.
12. The antigen binding protein of embodiment 9, 10 or 11, wherein the antigen binding protein forms part of a Chimeric antigen receptor (CAR) or T cell receptor (TCR).
13. The antigen binding protein of any one of embodiments 9 to 12, wherein the antigen binding protein comprising a first antigen binding domain and a second binding domain, wherein the self-regulation element is positioned between the first and second antigen binding domains.
14. The antigen binding protein of embodiment 13, wherein the first antigen binding domain and second binding domain each comprise a light chain variable domain and a heavy chain variable domain, wherein the light chain variable domain comprises a light chain complementarity determining region (LCDR)1, an LCDR2 and an LCDR3, and wherein the heavy chain variable domain comprises a heavy chain complementarity determining region (HCDR)1, an HCDR2 and an HCDR3. 15. The antigen binding protein of embodiment 13 or 14, wherein the first antigen binding domain and second binding domain comprise identical CDRs, comprise biparatopic CDRs, or comprise bi-specific CDRs.
16. The antigen binding protein of any one of embodiments 13 to 15, wherein the antigen binding protein is an antibody or fragment thereof.
17. The antigen binding protein of any one of embodiments 9 to 16, wherein the antigen binding protein comprises a single domain fragment, a Fab fragment, a Fab′ fragment, a F(ab)′2 fragment, a single chain Fab (scFab) fragment, a single chain Fv protein (scFv), a tandem scFv protein, a disulfide stabilized Fv protein (dsFv), or a scFv-Fc protein.
18. The antigen binding protein of any one of embodiments 9 to 17, wherein the antigen binding protein is included in a monoclonal antibody, bispecific antibody, or a bi-specific T cell engager.
19. The antigen binding protein of any one of embodiments 9 to 18, wherein the first and second antigen binding domains are scFv proteins covalently linked by a peptide linker.
20. The antigen binding protein of any one of embodiments 9 to 19, wherein the self-regulation element is present within a peptide linker.
21. The antigen binding protein of any one of embodiments 9 to 20, wherein the self-regulation element comprises a Granzyme B cleavage site and the endogenous factor is Granzyme B.
22. The antigen binding protein of any one of embodiments 9 to 21, wherein the first antigen binding domain selectively binds to Receptor Tyrosine Kinase Like Orphan Receptor 1 (ROR1).
23. The antigen binding protein of embodiment 22, wherein the first antigen binding domain comprises a light chain variable domain and a heavy chain variable domain wherein the light chain variable domain comprises a light chain complementarity determining region (LCDR)1, an LCDR2 and an LCDR3, wherein LCDR1 comprises an amino acid sequence set forth in SEQ ID NO: 19; LCDR2 comprises the amino acid sequence set forth in SEQ ID NO: 20; and LCDR3 comprises the amino acid sequence set forth in SEQ ID NO: 21; and wherein the heavy chain variable domain comprises a heavy chain complementarity determining region (HCDR)1, an HCDR2 and an HCDR3, wherein HCDR1 comprises the amino acid sequence set forth in SEQ ID NO: 22; HCDR2 comprises the amino acid sequence set forth in SEQ ID NO: 23; and HCDR3 comprises the amino acid sequence set forth in SEQ ID NO: 24; wherein the sequence of each complementarity determining region may differ from the given sequence at up to two amino acid positions.
24. The antigen binding protein of embodiment 23, wherein:
   (a) the light chain variable domain comprises the amino acid sequence as set forth as SEQ ID NO: 29 and the heavy chain variable domain comprises the amino acid sequence as set forth as SEQ ID NO: 35;
   (b) the light chain variable domain comprises the amino acid sequence as set forth as SEQ ID NO: 30 and the heavy chain variable domain comprises the amino acid sequence as set forth as SEQ ID NO: 36;
   (c) the light chain variable domain comprises the amino acid sequence as set forth as SEQ ID NO: 31 and the heavy chain variable domain comprises the amino acid sequence as set forth as SEQ ID NO: 37;
   (d) the light chain variable domain comprises the amino acid sequence as set forth as SEQ ID NO: 32 and the heavy chain variable domain comprises the amino acid sequence as set forth as SEQ ID NO: 38; or
   (e) the light chain variable domain comprises the amino acid sequence as set forth as SEQ ID NO: 33 and the heavy chain variable domain comprises the amino acid sequence as set forth as SEQ ID NO: 39,
   wherein each light chain variable domain and heavy chain variable domain above may have at least 90% identity to the amino acid sequence set forth above.
25. The antigen binding protein of any one of embodiments 22 to 24, wherein the second antigen binding domain selectively binds to the CD3 subunit of the T-Cell Receptor (TCR).
26. The antigen binding protein of any one of embodiments 22 to 25, wherein the Granzyme B cleavage site comprises SEQ ID NO: 7. 27. The antigen binding protein of any one of embodiments 9 to 20, wherein the self-regulation element comprises a Thrombin cleavage site and the endogenous factor is Thrombin.
28. The antigen binding protein of any one of embodiments 9 to 20 and 27, wherein the first antigen binding domain selectively binds to FIXa/FIX and the second antigen binding domain selectively binds to FX/FXa.
29. The antigen binding protein of any one of embodiments 9 to 20 and 27 or 28, wherein the first antigen binding domain and second binding domain each comprise a light chain variable domain and a heavy chain variable domain, wherein the light chain variable domain comprises a light chain complementarity determining region (LCDR)1, an LCDR2 and an LCDR3, and wherein the heavy chain variable domain comprises a heavy chain complementarity determining region (HCDR)1, an HCDR2 and an HCDR3;
   wherein for the first antigen binding domain LCDR1 comprises the amino acid sequence set forth in SEQ ID NO: 63; LCDR2 comprises the amino acid sequence set forth in SEQ ID NO: 64; and LCDR3 comprises the amino acid sequence set forth in SEQ ID NO: 65; and wherein the heavy chain variable domain comprises a heavy chain complementarity determining region (HCDR)1, an HCDR2 and an HCDR3, wherein HCDR1 comprises the amino acid sequence set forth in SEQ ID NO: 66; HCDR2 comprises the amino acid sequence set forth in SEQ ID NO: 67; and HCDR3 comprises the amino acid sequence set forth in SEQ ID NO:68; and
   wherein for the second antigen binding domain LCDR1 comprises the amino acid sequence set forth in SEQ ID NO: 63; LCDR2 comprises the amino acid sequence set forth in SEQ ID NO: 64; and LCDR3 comprises the amino acid sequence set forth in SEQ ID NO: 65; and wherein the heavy chain variable domain comprises a heavy chain complementarity determining region (HCDR)1, an HCDR2 and an HCDR3, wherein HCDR1 comprises the amino acid sequence set forth in SEQ ID NO: 69; HCDR2 comprises the amino acid sequence set forth in SEQ ID NO: 70; and HCDR3 comprises the amino acid sequence set forth in SEQ ID NO: 71.
30. The antigen binding protein of embodiment 29, wherein for the first antigen binding domain the light chain variable domain comprises the amino acid sequence as set forth as SEQ ID NO: 72 and the heavy chain variable domain comprises the amino acid sequence as set forth as SEQ ID NO: 73, and for the second antigen binding domain the light chain variable domain comprises the amino acid sequence as set forth as SEQ ID NO: 72 and the heavy chain variable domain comprises the amino acid sequence as set forth as SEQ ID NO: 74.
31. The antigen binding protein of any one of embodiments 9 to 20 and 27 to 30, wherein the Thrombin cleavage site comprises SEQ ID NO: 1.
32. The antigen binding protein of any one of embodiments 9 to 31, wherein the antigen binding protein is labelled.
33. The antigen binding protein of embodiment 32, wherein the label is a fluorescent, an enzymatic, or a radioactive label.
34. A composition comprising the therapeutic agent of any one of embodiments 1 to 33 and a pharmaceutically acceptable carrier.
35. An isolated nucleic acid molecule encoding the antigen binding protein of any one of embodiments 8 to 31.
36. The isolated nucleic acid molecule of embodiment 35, operably linked to a promoter.
37. An expression vector comprising the isolated nucleic acid molecule of embodiment 35 or embodiment 36.
38. An isolated host cell transformed with the nucleic acid molecule of embodiment 35 or 36, or the vector of embodiment 37.
39. The antigen binding protein of any one of embodiments 9 to 31, wherein the antigen binding protein is exogenously expressed in a CAR-T cell, a CAR-M cell or a modified NK cell.
40. A method of treatment of Haemophilia A, comprising administering the antigen binding protein of any one of embodiments 27 to 31, or the composition of embodiment 34, to an individual in need thereof.
41. A method of prevention of pro-thrombotic risks, comprising administering the antigen binding protein of any one of embodiments 27 to 31, or the composition of embodiment 34, to an individual in need thereof.
42. A method of treatment of cancer, comprising administering the antigen binding protein of any one of embodiments 20 to 26, or the composition of embodiment 34, to an individual in need thereof.
43. The method of embodiment 42, wherein the cancer is leukaemia, pancreatic cancer, prostate cancer, colon cancer, bladder cancer, ovarian cancer, glioblastoma, testicular cancer, uterine cancer, adrenal cancer, breast cancer, lung cancer, melanoma, neuroblastoma, sarcoma or renal cancer.
44. A method of prevention of mode-of-action (MoA)-related toxicities associated with immunotherapies, comprising administering the antigen binding protein of any one of embodiments 20 to 26, or the composition of embodiment 34, to an individual in need thereof.
45. A method of prevention of cytokine release syndrome, comprising administering the antigen binding protein of any one of embodiments 20 to 26, or the composition of embodiment 34, to an individual in need thereof.

### REFERENCES

Ferriere, S., I. Peyron, O. D. Christophe, C. Kawecki, C. Casari, V. Muczynski, A. C. Nathwani, A. Kauskot, P. J. Lenting and C. V. Denis (2020). “A hemophilia A mouse model for the in vivo assessment of Emicizumab function.” Blood.
Gallwitz, M., M. Enoksson, M. Thorpe and L. Hellman (2012). “The extended cleavage specificity of human thrombin.” PLoS One 7(2): e31756.
Harris et al. Definition and redesign of the extended substrate specificity of granzyme B, (1998) J Biol Chem: 273 pp. 27364-73.
Hartmann, R., T. Feenstra, L. Valentino, M. Dockal and F. Scheiflinger (2018). “In vitro studies show synergistic effects of a procoagulant bispecific antibody and bypassing agents.” J Thromb Haemost.
Kochenderfer, J. N., R. P. T. Somerville, T. Lu, V. Shi, A. Bot, J. Rossi, A. Xue, S. L. Goff, J. C. Yang, R. M. Sherry, C. A. Klebanoff, U. S. Kammula, M. Sherman, A. Perez, C. M. Yuan, T. Feldman, J. W. Friedberg, M. J. Roschewski, S. A. Feldman, L. McIntyre, M. A. Toomey and S. A. Rosenberg (2017). “Lymphoma Remissions Caused by Anti-CD19 Chimeric Antigen Receptor T Cells Are Associated With High Serum Interleukin-15 Levels.” J Clin Oncol 35(16): 1803-1813.
Lenting, P. J., C. V. Denis and O. D. Christophe (2017). “Emicizumab, a bispecific antibody recognizing coagulation factors IX and X: how does it actually compare to factor VIII?” Blood 130(23): 2463-2468.
Wee, L. J., E. P. Er, L. F. Ng and J. C. Tong (2011). “In silico prediction of the granzyme B degradome.” BMC Genomics 12 Suppl 3: S11.

1. A therapeutic agent comprising an autoregulation element, wherein the autoregulation element is a small peptide cleavable by an endogenous factor, wherein the activity of the endogenous factor is modified as a consequence of the activity of the therapeutic agent, and wherein cleavage of the autoregulation element results in separation of the therapeutic agent into non-functional fragments.

2. The therapeutic agent of claim 1, wherein the endogenous factor is active in the same metabolic pathway as that acted on by the therapeutic agent, optionally, wherein the endogenous factor is a protease.

3. The therapeutic agent of claim 1 or claim 2, wherein the autoregulation element comprises a proteolytic cleavage site.

4. The therapeutic agent of any one of the preceding claims, wherein the rate of cleavage of the autoregulation element by the endogenous factor is correlated with the level of activity of the therapeutic agent, optionally, wherein the rate of cleavage of the autoregulation element by the endogenous factor is correlated with the level of activity of the therapeutic agent and the peptide sequence of the autoregulation element.

5. The therapeutic agent of any one of claims 1 to 4, wherein autoregulation element comprises a thrombin or Granzyme B cleavage recognition site.

6. The therapeutic agent of any one of claims 1 to 5, wherein the therapeutic agent is an antigen binding protein comprising a first antigen binding domain, wherein the first antigen binding domain comprises a heavy chain variable domain comprising a heavy chain complementarity determining region (HCDR)1, an HCDR2 and an HCDR3, and wherein the autoregulation element is positioned C-terminal to the first antigen binding domain.

7. The antigen binding protein of claim 6, wherein the antigen binding protein comprising a first antigen binding domain, wherein the first antigen binding domain comprises a light chain variable domain and a heavy chain variable domain, wherein the light chain variable domain comprises a light chain complementarity determining region (LCDR)1, an LCDR2 and an LCDR3, and wherein the heavy chain variable domain comprises a heavy chain complementarity determining region (HCDR)1, an HCDR2 and an HCDR3, and wherein the autoregulation element is positioned C-terminal to the first antigen binding domain.

8. The antigen binding protein of any one of the preceding claims, wherein the antigen binding protein comprising a first antigen binding domain and a second binding domain, wherein the autoregulation element is positioned between the first and second antigen binding domains.

9. The antigen binding protein of claim 8, wherein the first antigen binding domain and second binding domain each comprise a light chain variable domain and a heavy chain variable domain, wherein the light chain variable domain comprises a light chain complementarity determining region (LCDR)1, an LCDR2 and an LCDR3, and wherein the heavy chain variable domain comprises a heavy chain complementarity determining region (HCDR)1, an HCDR2 and an HCDR3.

10. The antigen binding protein of claim 8 or claim 9, wherein the antigen binding protein is an antibody or fragment thereof, optionally wherein the antigen binding protein comprises a single domain fragment, a Fab fragment, a Fab′ fragment, a F(ab)′2 fragment, a single chain Fab (scFab) fragment, a single chain Fv protein (scFv), a tandem scFv protein, a disulfide stabilized Fv protein (dsFv), or a scFv-Fc protein.

11. The antigen binding protein of any one of claims 6 to 10, wherein the autoregulation element is present within a peptide linker.

12. The antigen binding protein of any one of claims 6 to 11, wherein the autoregulation element comprises a Granzyme B cleavage site and the endogenous factor is Granzyme B, optionally wherein the first antigen binding domain selectively binds to Receptor Tyrosine Kinase Like Orphan Receptor 1 (ROR1).

13. The antigen binding protein of claim 12, wherein the first antigen binding domain comprises a light chain variable domain and a heavy chain variable domain wherein the light chain variable domain comprises a light chain complementarity determining region (LCDR)1, an LCDR2 and an LCDR3, wherein LCDR1 comprises an amino acid sequence set forth in SEQ ID NO: 19; LCDR2 comprises the amino acid sequence set forth in SEQ ID NO: 20; and LCDR3 comprises the amino acid sequence set forth in SEQ ID NO: 21; and wherein the heavy chain variable domain comprises a heavy chain complementarity determining region (HCDR)1, an HCDR2 and an HCDR3, wherein HCDR1 comprises the amino acid sequence set forth in SEQ ID NO: 22; HCDR2 comprises the amino acid sequence set forth in SEQ ID NO: 23; and HCDR3 comprises the amino acid sequence set forth in SEQ ID NO: 24; wherein the sequence of each complementarity determining region may differ from the given sequence at up to two amino acid positions.

14. The antigen binding protein of claim 13, wherein the first antigen binding domain is comprised in a chimeric antigen receptor (CAR).

15. The antigen binding protein of claim 13, wherein the second antigen binding domain selectively binds to the CD3 subunit of the T-Cell Receptor (TCR).

16. The antigen binding protein of any one of claim 13 to 15, wherein the Granzyme B cleavage site comprises SEQ ID NO: 7.

17. The antigen binding protein of any one of claims 6 to 11, wherein the autoregulation element comprises a Thrombin cleavage site and the endogenous factor is Thrombin, optionally, wherein the first antigen binding domain selectively binds to FIXa/FIX and the second antigen binding domain selectively binds to FX/FXa.

18. The antigen binding protein of any one of claims 6 to 11 and 17, wherein the first antigen binding domain and second binding domain each comprise a light chain variable domain and a heavy chain variable domain, wherein the light chain variable domain comprises a light chain complementarity determining region (LCDR)1, an LCDR2 and an LCDR3, and wherein the heavy chain variable domain comprises a heavy chain complementarity determining region (HCDR)1, an HCDR2 and an HCDR3;
wherein for the first antigen binding domain LCDR1 comprises the amino acid sequence set forth in SEQ ID NO: 63; LCDR2 comprises the amino acid sequence set forth in SEQ ID NO: 64; and LCDR3 comprises the amino acid sequence set forth in SEQ ID NO: 65; and wherein the heavy chain variable domain comprises a heavy chain complementarity determining region (HCDR)1, an HCDR2 and an HCDR3, wherein HCDR1 comprises the amino acid sequence set forth in SEQ ID NO: 66; HCDR2 comprises the amino acid sequence set forth in SEQ ID NO: 67; and HCDR3 comprises the amino acid sequence set forth in SEQ ID NO:68; and
wherein for the second antigen binding domain LCDR1 comprises the amino acid sequence set forth in SEQ ID NO: 63; LCDR2 comprises the amino acid sequence set forth in SEQ ID NO: 64; and LCDR3 comprises the amino acid sequence set forth in SEQ ID NO: 65; and wherein the heavy chain variable domain comprises a heavy chain complementarity determining region (HCDR)1, an HCDR2 and an HCDR3, wherein HCDR1 comprises the amino acid sequence set forth in SEQ ID NO: 69; HCDR2 comprises the amino acid sequence set forth in SEQ ID NO: 70; and HCDR3 comprises the amino acid sequence set forth in SEQ ID NO: 71.

19. The antigen binding protein of any one of claims 6 to 11 and 17 or 18, wherein the Thrombin cleavage site comprises SEQ ID NO: 1.

20. A composition comprising the therapeutic agent of any one of claims 1 to 19 and a pharmaceutically acceptable carrier.

21. An isolated nucleic acid molecule encoding the antigen binding protein of any one of claims 6 to 19, optionally operably linked to a promoter.

22. An isolated host cell transformed with the nucleic acid molecule of claim 21.

23. A method of treatment of Haemophilia A, comprising administering the antigen binding protein of any one of claims 17 to 19, or the composition of claim 20, to an individual in need thereof.

24. A method of prevention of pro-thrombotic risks, comprising administering the antigen binding protein of any one of claims 17 to 19, or the composition of claim 20, to an individual in need thereof.

25. A method of treatment of cancer, comprising administering the antigen binding protein of any one of claims 11 to 16, or the composition of claim 20, to an individual in need thereof.

26. A method of prevention of mode-of-action (MoA)-related toxicities associated with immunotherapies, comprising administering the antigen binding protein of any one of claims 11 to 16, or the composition of claim 20, to an individual in need thereof.

27. A method of prevention of cytokine release syndrome, comprising administering the antigen binding protein of any one of claims 11 to 16, or the composition of claim 20, to an individual in need thereof.


SEQUENCE LISTING (84 sequences)

SEQ ID NO: 1 - protein, 8 aa, synthetic construct
LTPRGVRL

SEQ ID NO: 2 - protein, 8 aa, synthetic construct
LTPRLVRL

SEQ ID NO: 3 - protein, 8 aa, synthetic construct
LTPRDVRL

SEQ ID NO: 4 - protein, 8 aa, synthetic construct
WYLRSNNG

SEQ ID NO: 5 - protein, 8 aa, synthetic construct
IEPDSESQ

SEQ ID NO: 6 - protein, 8 aa, synthetic construct
IEADSESQ

SEQ ID NO: 7 - protein, 8 aa, synthetic construct
IEPDSLEE

SEQ ID NO: 8 - protein, 20 aa, synthetic construct
HSRLGRIEADSESQEDIIRN

SEQ ID NO: 9 - protein, 8 aa, synthetic construct
VEPDSLEE

SEQ ID NO: 10 - protein, 8 aa, synthetic construct
LEPDSLEE

SEQ ID NO: 11 - protein, 8 aa, synthetic construct
IEGDSLEE

SEQ ID NO: 12 - protein, 8 aa, synthetic construct
IEADSLEE

SEQ ID NO: 13 - protein, 8 aa, synthetic construct
IEPDALEE

SEQ ID NO: 14 - protein, 8 aa, synthetic construct
IEPDSEEQ

SEQ ID NO: 15 - protein, 8 aa, synthetic construct
IEPDSEVE

SEQ ID NO: 16 - protein, 8 aa, synthetic construct
IEPNSLEE

SEQ ID NO: 17 - protein, 8 aa, synthetic construct
IEPESLEE

SEQ ID NO: 18 - protein, 8 aa, synthetic construct
IEPQSLEE

SEQ ID NO: 19 - protein, 6 aa, synthetic construct
QNIDRY

SEQ ID NO: 20 - skipped

SEQ ID NO: 21 - protein, 9 aa, synthetic construct
LQYNSLPLT

SEQ ID NO: 22 - protein, 8 aa, synthetic construct
GFIFSEHN

SEQ ID NO: 23 - protein, 8 aa, synthetic construct
ISDDGRNT

SEQ ID NO: 24 - protein, 10 aa, synthetic construct
  VARIANT 1: note = where X is A or T
  VARIANT 2: note = where X is S, K or R
XXHRYNLFDS

SEQ ID NO: 25 - protein, 10 aa, synthetic construct
ASHRYNLFDS

SEQ ID NO: 26 - protein, 10 aa, synthetic construct
TSHRYNLFDS

SEQ ID NO: 27 - protein, 10 aa, synthetic construct
AKHRYNLFDS

SEQ ID NO: 28 - protein, 10 aa, synthetic construct
ARHRYNLFDS

SEQ ID NO: 29 - protein, 107 aa, synthetic construct
  REGION 37..42: note = CDRs
  REGION 50..52: note = CDRs
  REGION 89..97: note = CDRs
DIQMTQSPSFLSASVGDRVTINCKASQNIDRYLNWYQQKLGEAPKRLLYNTNKLQTGIPSRFSGSGSATDFTLTISSLQPEDFATYFCLQYNSLPLTFGSGTKLEIK

SEQ ID NO: 30 - protein, 107 aa, synthetic construct
DIQMTQSPSSLSASVGDRVTITCKASQNIDRYLNWYQQKPGKAPKRLIYNTNKLQTGVPSRFSGSGSGTEFTLTISSLQPEDFATYYCLQYNSLPLTFGQGTKLEIK

SEQ ID NO: 31 - protein, 107 aa, synthetic construct
DIQMTQSPSSLSASVGDRVTITCKASQNIDRYLNWFQQKPGKAPKSLIYNTNKLQTGVPSKFSGSGSGTDFTLTISSLQPEDFATYYCLQYNSLPLTFGQGTRLEIK

SEQ ID NO: 32 - protein, 107 aa, synthetic construct
DIQMTQSPSSLSASVGDRVTITCKASQNIDRYLNWYQQKPGKAPKLLIYNTNKLQTGVPSRFSGSGSGTDFTLTISSLQPEDFATYYCLQYNSLPLTFGQGTKLEIK

SEQ ID NO: 33 - protein, 107 aa, synthetic construct
DIQLTQSPSFLSASVGDRVTITCKASQNIDRYLNWYQQKPGKAPKLLIYNTNKLQTGVPSRFSGSGSGTEFTLTISSLQPEDFATYYCLQYNSLPLTFGQGTKLEIK

SEQ ID NO: 34 - protein, 107 aa, synthetic construct
DIQMTQSPSTLSASVGDRVTITCKASQNIDRYLNWYQQKPGKAPKLLIYNTNKLQTGVPSRFSGSGSGTEFTLTISSLQPDDFATYYCLQYNSLPLTFGQGTKLEIK

SEQ ID NO: 35 - protein, 117 aa, synthetic construct
EVQLVESGGGLVQPGRSLKLSCAASGFIFSEHNMAWVRQAPKKGLEWVATISDDGRNTYYRDSMRGRFTISRENARSTLYLQLDSLRSEDTATYYCASHRYNLFDSWGQGVMVTVSS

SEQ ID NO: 36 - protein, 117 aa, synthetic construct
QVQLVESGGGVVQPGRSLRLSCAASGFIFSEHNMAWVRQAPGKGLEWVATISDDGRNTYYRDSMRGRFTISRDNSKNTLYLQMNSLRAEDTAVYYCTSHRYNLFDSWGQGTMVTVSS

SEQ ID NO: 37 - protein, 117 aa, synthetic construct
EVQLVESGGGLVQPGGSLRLSCAASGFIFSEHNMAWVRQAPGKGLEWVSTISDDGRNTYYRDSMRGRFTISRDNSKNTLYLQMNSLRAEDTAVYYCAKHRYNLFDSWGQGTLVTVSS

SEQ ID NO: 38 - protein, 117 aa, synthetic construct
EVQLVESGGGLVQPGGSLRLSCAASGFIFSEHNMAWVRQAPGKGLEWVATISDDGRNTYYRDSMRGRFTISRDNAKNSLYLQMNSLRAEDTAVYYCARHRYNLFDSWGQGTMVTVSS

SEQ ID NO: 39 - protein, 117 aa, synthetic construct
EVQLVESGGGLVQPGGSLRLSCAASGFIFSEHNMAWVRQAPGKGLVWVSTISDDGRNTYYRDSMRGRFTISRDNAKNTLYLQMNSLRAEDTAVYYCARHRYNLFDSWGQGTLVTVSS

SEQ ID NO: 40 - protein, 117 aa, synthetic construct
EVQLVESGGGLVQPGRSLRLSCAASGFIFSEHNMAWVRQAPGKGLEWVSTISDDGRNTYYRDSMRGRFTISRDNAKNSLYLQMNSLRAEDTALYYCAKHRYNLFDSWGQGTLVTVSS

SEQ ID NO: 41 - protein, 106 aa, synthetic construct [sequence omitted]

SEQ ID NO: 42 - protein, 106 aa, synthetic construct
EIVLTQSPATLSLSPGERATLSCSASSSVSYMNWYQQKPGQAPRLLIYDTSKLASGIPARFSGSGSGTDFTLTISSLEPEDFAVYYCQQWSSNPFTFGQGTKLEIK

SEQ ID NO: 43 - protein, 106 aa, synthetic construct
DIQMTQSPSSLSASVGDRVTITCSASSSVSYMNWYQQKPGKAPKLLIYDTSKLASGVPSRFSGSGSGTDFTLTISSLQPEDFATYYCQQWSSNPFTFGQGTKLEIK

SEQ ID NO: 44 - protein, 106 aa, synthetic construct
DIQLTQSPSFLSASVGDRVTITCSASSSVSYMNWYQQKPGKAPKLLIYDTSKLASGVPSRFSGSGSGTEFTLTISSLQPEDFATYYCQQWSSNPFTFGQGTKLEIK

SEQ ID NO: 45 - protein, 106 aa, synthetic construct
DIQMTQSPSTLSASVGDRVTITCSASSSVSYMNWYQQKPGKAPKLLIYDTSKLASGVPSRFSGSGSGTEFTLTISSLQPDDFATYYCQQWSSNPFTFGQGTKLEIK

SEQ ID NO: 46 - protein, 106 aa, synthetic construct
DIQMTQSPSSLSASVGDRVTITCSASSSVSYMNWYQQKPGKAPKRLIYDTSKLASGVPSRFSGSGSGTEFTLTISSLQPEDFATYYCQQWSSNPFTFGQGTKVEIK

SEQ ID NO: 47 - protein, 119 aa, synthetic construct [sequence omitted]

SEQ ID NO: 48 - protein, 119 aa, synthetic construct
QVQLVQSGAEVKKPGASVKVSCKASGYTFTRYTMHWVRQAPGQGLEWMGYINPSRGYTNYNQKFKDRVTMTRDTSTSTVYMELSSLRSEDTAVYYCARYYDDHYCLDYWGQGTLVTVSS

SEQ ID NO: 49 - protein, 119 aa, synthetic construct
QVQLVQSGAEVKKPGASVKVSCKASGYTFTRYTMHWVRQAPGQRLEWMGYINPSRGYTNYNQKFKDRVTITRDTSASTAYMELSSLRSEDTAVYYCARYYDDHYCLDYWGQGTLVTVSS

SEQ ID NO: 50 - protein, 119 aa, synthetic construct
QVQLVQSGAEVKKPGSSVKVSCKASGYTFTRYTMHWVRQAPGQGLEWMGYINPSRGYTNYNQKFKDRVTITADKSTSTAYMELSSLRSEDTAVYYCARYYDDHYCLDYWGQGTMVTVSS

SEQ ID NO: 51 - protein, 119 aa, synthetic construct
QVQLVESGGGLVKPGGSLRLSCAASGYTFTRYTMHWIRQAPGKGLEWVSYINPSRGYTNYNQKFKDRFTISRDNAKNSLYLQMNSLRAEDTAVYYCARYYDDHYCLDYWGQGTTVTVSS

SEQ ID NO: 52 - protein, 119 aa, synthetic construct
EVQLVESGGGLVQPGGSLRLSCAASGYTFTRYTMHWVRQAPGKGLEWVSYINPSRGYTNYNQKFKDRFTISRDNSKNTLYLQMNSLRAEDTAVYYCAKYYDDHYCLDYWGQGTLVTVSS

SEQ ID NO: 53 - protein, 10 aa, synthetic construct
SASSSVSYMN

SEQ ID NO: 54 - protein, 7 aa, synthetic construct
DTSKLAS

SEQ ID NO: 55 - protein, 9 aa, synthetic construct
QQWSSNPFT

SEQ ID NO: 56 - protein, 10 aa, synthetic construct
GYTFTRYTMH

SEQ ID NO: 57 - protein, 17 aa, synthetic construct
YINPSRGYTNYNQKFKD

SEQ ID NO: 58 - protein, 10 aa, synthetic construct
YYDDHYCLDY

SEQ ID NO: 59 - protein, 5 aa, synthetic construct
GGGGS

SEQ ID NO: 60 - protein, 10 aa, synthetic construct
GGGGSGGGGS

SEQ ID NO: 61 - protein, 490 aa, synthetic construct
QVQLVESGGGVVQPGRSLRLSCAASGFIFSEHNMAWVRQAPGKGLEWVATISDDGRNTYYRDSMRGRFTISRDNSKNTLYLQMNSLRAEDTAVYYCTSHRYNLFDSWGQGTMVTVSSGGGGSGGGGSGGGGSDIQMTQSPSSLSASVGDRVTITCKASQNIDRYLNWYQQKPGKAPKRLIYNTNKLQTGVPSRFSGSGSGTEFTLTISSLQPEDFATYYCLQYNSLPLTFGQGTKLEIKSGSGGGGSQVQLVQSGAEVKKPGSSVKVSCKASGYTFTRYTMHWVRQAPGQGLEWMGYINPSRGYTNYNQKFKDRVTITADKSTSTAYMELSSLRSEDTAVYYCARYYDDHYCLDYWGQGTMVTVSSVEGGSGGSGGSGGSGGVDDIQMTQSPSSLSASVGDRVTITCSASSSVSYMNWYQQKPGKAPKRLIYDTSKLASGVPSRFSGSGSGTEFTLTISSLQPEDFATYYCQQWSSNPFTFGQGTKVEIK

SEQ ID NO: 62 - protein, 496 aa, synthetic construct
QVQLVESGGGVVQPGRSLRLSCAASGFIFSEHNMAWVRQAPGKGLEWVATISDDGRNTYYRDSMRGRFTISRDNSKNTLYLQMNSLRAEDTAVYYCTSHRYNLFDSWGQGTMVTVSSGGGGSGGGGSGGGGSDIQMTQSPSSLSASVGDRVTITCKASQNIDRYLNWYQQKPGKAPKRLIYNTNKLQTGVPSRFSGSGSGTEFTLTISSLQPEDFATYYCLQYNSLPLTFGQGTKLEIKSGSGGGGSQVQLVQSGAEVKKPGSSVKVSCKASGYTFTRYTMHWVRQAPGQGLEWMGYINPSRGYTNYNQKFKDRVTITADKSTSTAYMELSSLRSEDTAVYYCARYYDDHYCLDYWGQGTMVTVSSVEGGSGGSGGSGGSGGVDDIQMTQSPSSLSASVGDRVTITCSASSSVSYMNWYQQKPGKAPKRLIYDTSKLASGVPSRFSGSGSGTEFTLTISSLQPEDFATYYCQQWSSNPFTFGQGTKVEIKHHHHHH

SEQ ID NO: 63 - protein, 6 aa, synthetic construct
RNIERQ

SEQ ID NO: 64 - skipped

SEQ ID NO: 65 - protein, 9 aa, synthetic construct
QQYSDPPLT

SEQ ID NO: 66 - protein, 8 aa, synthetic construct
GFTFSYYD

SEQ ID NO: 67 - protein, 8 aa, synthetic construct
ISPSGQST

SEQ ID NO: 68 - protein, 16 aa, synthetic construct
ARRTGREYGGGWYFDY

SEQ ID NO: 69 - protein, 8 aa, synthetic construct
GYTFTDNN

SEQ ID NO: 70 - protein, 8 aa, synthetic construct
INTRSGGS

SEQ ID NO: 71 - protein, 12 aa, synthetic construct
ARRKSYGYYLDE

SEQ ID NO: 72 - protein, 107 aa, synthetic construct
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIK

SEQ ID NO: 73 - protein, 123 aa, synthetic construct
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSS

SEQ ID NO: 74 - protein, 119 aa, synthetic construct
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSS

SEQ ID NO: 75 - protein, 12 aa, synthetic construct
ESKYGPPCPPCP

SEQ ID NO: 76 - protein, 20 aa, synthetic construct
ESKLTPRGVRLYGPPCPPCP

SEQ ID NO: 77 - protein, 7 aa, synthetic construct
GSGGGGS

SEQ ID NO: 78 - protein, 18 aa, synthetic construct
  REGION 6..13: note = granzyme B cleavage site
GGGGSXXXXXXXXGGGGS

SEQ ID NO: 79 - protein, 18 aa, synthetic construct
GGGGSIEPDSESQGGGGS

SEQ ID NO: 80 - protein, 18 aa, synthetic construct
GGGGSIEADSESQGGGGS

SEQ ID NO: 81 - protein, 18 aa, synthetic construct
GGGGSIEPDSLEEGGGGS

SEQ ID NO: 82 - protein, 15 aa, synthetic construct
EPKSPDKTHTCPPCP

SEQ ID NO: 83 - protein, 18 aa, synthetic construct
IEPDSLEEDKTHTCPPCP

SEQ ID NO: 84 - protein, 23 aa, synthetic construct
IEPDSLEEEPKSPDKTHTCPPCP